(19) United States
(12) Patent Application Publication
(10) Pub. No.: US 2026/0245667 A1
(43) Pub. Date: Aug. 20, 2026

(54) EFFICIENT DETECTION OF DECENTRALIZED BIOMARKERS OF GROUPS OF DNA SEQUENCES IN MICROBIOME DNA

(71) Applicant: ALPHABIOME AI LTD, Ramat Yishai (IL)

(72) Inventor: Yaniv ALTSHULER, Ramat Yishay (IL)

(73) Assignee: ALPHABIOME AI LTD, Ramat Yishai (IL)

(21) Appl. No.: 19/529,600

(22) Filed: Feb. 4, 2026

Related U.S. Application Data
(63) Continuation of application No. PCT/IL2024/050854, filed on Aug. 25, 2024.
(60) Provisional application No. 63/537,259, filed on Sep. 8, 2023.

(51) Int. Cl. G16B 40/20; G16B 5/00

(52) U.S. Cl. CPC G16B 40/20; G16B 5/00

(57) ABSTRACT

Detecting biomarkers in microbiome DNA to predict a biological condition. A microbiome network may be generated comprising nodes representing microbiome DNA sequences and edges representing a co-occurrence of each pair of microbiome DNA sequences in a same DNA sample or sub-length. Nodes may be bundled into distinct groups based on the node's degree quantifying a number of its edges indicating a number of unique microbiome DNA sequences that co-occur with the microbiome DNA sequence represented by the node in the same DNA sample or sub-length. Groups of bundled microbiome DNA sequences may be validated having an internal connectivity that satisfies an anomaly condition indicating the microbiome DNA sequences of that group co-occur with a probability that is unlikely randomly statistical, e.g., that does not follow a power law distribution. A machine learning model may be trained with the validated groups to predict a biological condition correlated therewith.

### CROSS-REFERENCE TO RELATED APPLICATIONS

This application is a continuation of PCT International Application No. PCT/IL2024/050854, International Filing Date Aug. 25, 2024, claiming the benefit of U.S. Provisional Patent Application No. 63/537,259, filed Sep. 8, 2023, both of which are hereby incorporated by reference in their entireties.

### INCORPORATION BY REFERENCE OF SEQUENCE LISTING

The instant application contains a Sequence Listing conforming the rules of WIPO Standard ST.26 which has been submitted electronically in XML format and is hereby incorporated by reference in its entirety. The XML copy, created on Apr. 1, 2026, is named “P-628851-US-SQL-01APR26.xml”, and is 245,975 bytes in size.

### FIELD OF THE INVENTION

Embodiments of the present invention relate generally to the field of microbiome DNA. In particular, some embodiments of the invention relate to analyzing microbiome DNA to predict biological conditions (e.g., methane production, additive efficacy, dairy yield) in livestock (e.g., cows).

### BACKGROUND OF THE INVENTION

Ruminants, in an intricate symbiotic relationship to their resident microbiota, have the unique ability to breakdown complex polysaccharides like cellulose and hemi-cellulose, which constitute the primary components of their plant-based diet. This process is facilitated by the host animal's provision of a stable environment, facilitating continuous mixing, deconstruction, and fermentation of ingested plant material. This, in turn, results in the production of short-chain fatty acids which serve as a digestible energy source for the host animal.

The assembly and development of the rumen microbiota is a multifactorial process, influenced by several host and environmental factors. These include the host's age, diet, genetic makeup, and herd origin, all of which play a pivotal role in defining the microbiota's compositional layout. Moreover, the stochastic colonization events of the rumen during early life stages can leave lasting imprints on the ruminant microbiome's structure.

While this symbiotic relationship allows ruminants to thrive on fibrous diets, it also has an environmental cost. The ruminant digestive process is a significant contributor to the emission of methane, a potent greenhouse gas, which accounts for about 14% of total greenhouse emissions and has a global warming potential 28 times higher than carbon dioxide (CO₂). Notably, livestock are estimated to contribute to nearly 30% of all anthropogenic methane emissions.

Efforts to mitigate the environmental impact of dairy farming have given rise to several strategies. One such approach involves the utilization of microbial biomarkers to identify cows with high methane emission rates, thereby enabling targeted management strategies aimed at reducing methane emissions and fostering environmental sustainability. Another strategy characterizes microbial gene abundances as proxies for methane emissions, focusing specifically on metabolic pathways expected to exhibit variation between low and high methane emitters.

Correlating high methane emission rates with biomarkers, particularly in livestock microbiome DNA, presents a unique challenge because the biological effect of the constituent of the microbiome DNA sequences are largely unknown.

Microbiome DNA samples are sequenced into a plurality (e.g., tens to millions) of “reads,” each representing a continuous sequence of (e.g., fixed or variable length, such as, 100-150) nucleotides. Each read is then sub-divided into a plurality of “k-mers,” each representing relatively shorter continuous fixed or variable length sequences (e.g., a fixed-length integer number k, such as, 30; or variable length including k=30, 60, 72, etc.) of the read's nucleotides. For example, Each DNA sequence position can be one of four nucleotides (A, T, C, or G), so the total number of possible k-mers of length k in the microbiome DNA is 4^{k}. Experiments indicate k-mer lengths of 30-60 nucleotides associate with optimal phenotypic expression of biomarkers (e.g., shorter k-mer lengths often suffer higher false positives due to a higher likelihood of randomly appearing and longer k-mer lengths often suffer higher false negatives as longer sequences obfuscate or dilute significant segments). With 4³⁰ or more possible 30-mer combinations per DNA sample, there are too many k-mer combinations to practically model correlations between k-mers and biological effect. Groups of multiple k-mers, the combination of which often correlates with biological expression, compounds this problem as there are exponentially more combinations of k-mer groups than individual k-mers (e.g., for k-mers of length k, there are 2^{2*k} possible k-mers, approximately (2^{2*k})² possible pairs, and 2\hat{ }(2^{2*k}) possible groups of k-mers, which for k-mers of length k=30, would be 2⁶⁰=approximately 10¹⁸ possible k-mers and 2¹⁰⁰⁰⁰⁰⁰⁰⁰⁰⁰⁰⁰⁰⁰⁰⁰⁰⁰ possible k-mer groups). Such massive numbers of combinations of groups of k-mers makes it realistically impossible to model their correlation to biological effect.

Accordingly, there is longstanding need inherent in the art for, and a wealth of knowledge to be gained from, efficiently modelling the biological effect of groups of k-mer or other nucleotide sequences in microbiome DNA.

### SUMMARY OF THE INVENTION

Embodiments of the invention overcome this longstanding need inherent in the art by efficiently modeling correlations between biological conditions and groups of k-mer or other nucleotide sequences in microbiome DNA.

A system, device, method and non-transitory computer-readable storage medium comprising instructions that when executed cause one or more processors to accurately and efficiently detect biomarkers that are groups of DNA sequences in microbiome DNA of one or more hosts to predict a biological condition. A plurality of microbiome DNA sequences (e.g., DNA reads or k-mers) may be stored that are sequenced from microbiome DNA of one or more livestock hosts. A microbiome network may be generated comprising a plurality of nodes representing the respective plurality of microbiome DNA sequences and a plurality of edges representing a co-occurrence of each pair of microbiome DNA sequences in a same sample or sub-length of the microbiome DNA (e.g., reads in string or k-mers in reads). Microbiome DNA sequences may be bundled, into distinct (e.g., disjoint or overlapping) groups (e.g., candidate or potential anomalous groups), that are represented by nodes based on the node's degree, the degree of each node quantifying a number of edges that contain the node indicating a number of unique microbiome DNA sequences that co-occur with the microbiome DNA sequence represented bythe node in the same sample or sub-length of the microbiome DNA. Groups of microbiome DNA sequences may be validated as anomalous that each have an internal connectivity between nodes of the same group that satisfies an anomaly condition indicating the microbiome DNA sequences of that group co-occur with a probability that is unlikely randomly statistical. The internal connectivity condition is satisfied if the internal connectivity for a group of nodes deviates from an expected internal connectivity in a network that follows a power law distribution. A training dataset may be generated correlating the validated anomalous groups of microbiome DNA sequences from one or more livestock hosts with a biological condition measured in one or more same or different livestock hosts. A training phase may be executed in which a machine learning model may be trained with the training dataset to input microbiome DNA sequences from one or more livestock hosts and predict the biological condition for one or more same or different livestock hosts. A predictive run-time phase may then be executed in which the model inputs new microbiome DNA sequences from one or more hosts and outputs a prediction of the biological condition for one or more same or different hosts.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically illustrates a system for detecting biomarkers that are groups of DNA sequences in microbiome DNA of one or more hosts to predict a biological condition, according to an embodiment of the invention;

FIG. 2 schematically illustrates data structures representing a portion of a microbiome network of microbiome DNA sequences, according to an embodiment of the invention;

FIG. 3 is a graph of an adjacency matrix of a microbiome network clustered into groups, according to an embodiment of the invention;

FIG. 4 depicts two graphs of the relationship between a number of reads (y-axis) and a number of times those reads repeat (x-axis), according to an embodiment of the invention;

FIG. 5 includes two graphs representing distributions of repetitions of reads in a DNA sequence combining sequences sequenced from multiple biological samples from multiple hosts, according to an embodiment of the invention;

FIG. 6 includes two graphs representing distributions of repetitions of reads sequenced from the DNA of a single host obtained from a distinct biological sample, according to an embodiment of the invention;

FIG. 7 is a graph of an analysis of a clustered group of microbiome DNA sequences (the DNA sequences of the cluster of 32 k-mers shown are SEQ ID NOs: 219-250 going from the top to the bottom, respectively) labeled by circle “A” in FIG. 3, according to an embodiment of the invention;

FIG. 8 is a graph of an analysis of a clustered group of microbiome DNA sequences (the DNA sequences of the cluster of 25 k-mers shown are SEQ ID NOs: 251-275 going from the top to the bottom, respectively) labeled by circle “B” in FIG. 3, according to an embodiment of the invention;

FIG. 9 is a graph of two distributions related to a biological condition, according to an embodiment of the invention;

FIG. 10 schematically illustrates field test data structures, according to an embodiment of the invention;

FIG. 11 schematically illustrates data structures of an example first unsupervised learning stage of the two-stage technique for detecting biomarkers that are groups of microbiome DNA sequences, according to an embodiment of the invention;

FIG. 12 schematically illustrates data structures of an example second supervised learning stage of the two-stage technique for detecting biomarkers that are groups of microbiome DNA sequences, according to an embodiment of the invention;

FIG. 13 is a flowchart of a method for detecting biomarkers that are groups of microbiome DNA sequences of one or more hosts to predict a biological condition, according to an embodiment of the invention;

FIG. 14 is a graph of the sampling depth of sequenced samples across various sequencing processes, according to some embodiments of the invention;

FIG. 15 is an analysis of sequence duplication levels, illustrating the extent of duplication across various sequencing processes, according to some embodiments of the invention;

FIG. 16 is an image of the Geotech SEM5000 Methane Detector, used according to an embodiment of the invention;

FIG. 17 includes two graphs of methane levels for the same cow measured at different times by the SEM5000 sensor and the GreenFeed system, used according to an embodiment of the invention;

FIG. 18 is a graph comparing readings from the SEM5000 sensor and the LICOR 7810 laser-based sensor, used according to an embodiment of the invention; and

FIG. 19 is a graph showing a Bland-Altman analysis comparing the SEM5000 and the LICOR 7810 laser-based sensor, used according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In an era of increasing pressure to achieve sustainable farming, agriculturalists aim to optimize livestock feed for enhancing yield and minimizing environmental impact. Embodiments of the invention provide a new approach towards this goal by generating a machine learning model trained to analyze microbiome DNA sequences of biological samples of host(s) (e.g., dairy cattle) and predict correlations with biological conditions (e.g., the efficacy of feed additives) in host(s) that are trained based on the biological condition(s) measured in the host(s). In some embodiments, the microbiome-based predictive model may correlate specific microbiome groups with livestock production yield and methane emissions, allowing the prediction of feed additive efficacy. In general, the predictive model may correlate microbiome DNA groups with, and predict, any measurable biological condition(s) of the host(s), such as, feed, additive or medicinal efficacy, methane emissions, dairy or meat quality (e.g., protein content), composition or yield, gastrointestinal or overall health, disease susceptibility (e.g., to mouth to foot disease in cows, or diabetes in humans), disease tolerance, likelihood of disease recovery (e.g., from COVID), life expectancy, and/or fatality risk. Host(s) may include an individual host (e.g., a livestock animal, such as, a cow) or a group of multiple hosts (e.g., a livestock herd or genetic family). Predicting conditions for a herd or multiple hosts may be achieved by individually predicting conditions for each of the multiple hosts and averaging host-specific predictions (e.g., merging model outputs) or combining the DNA sequences of the multiple hosts in a “DNA soup” containing a combination of multiple or all hosts' samples (e.g., merging model inputs). Additionally or alternatively, each host's DNA sample may be represented by a network of nodes and DNA sequences from multiple hosts may be merged by super-positioning a plurality of networks each representing a distinct microbiome DNA sample to generate a composite network representing a plurality of microbiome DNA samples. Hosts sampled to generate training data inputs in the model's training phase may be the same or different than hosts for which biological conditions are predicted as outputs in the model's prediction phase. In the prediction phase, hosts sampled for the model's input data may be the same or different than hosts for which biological conditions are predicted as the model's output data. For example, hosts for which biological conditions are predicted may be in the same herd, family or have a similar genetic profile or sequences as the sampled hosts. Different hosts may be distinct (disjoint or overlapping) groups of one or more hosts.

Due to the unmanageable number of different possible k-mers in microbiome DNA (e.g., 4³⁰ 30-mer combinations) and even larger number of groups of those k-mers (e.g., 2^{4\hat{ }30} 30-mer pair combinations), conventional models cannot analyze these volumes of genetic material to predict their biological effect in practical time. Analyzing an exponential number of combinations of groups is an NP-hard (exponential) problem (e.g., similar to the traveling salesman problem) that is impractical to solve in finite time. Embodiments of the invention solve this problem by providing an efficient two-stage technique to predictively model groups of multiple k-mers to correlate, in combination, to biological condition(s) of microbiome hosts. In a first stage of unsupervised learning, microbiome DNA sequences are analyzed to efficiently validate significant, anomalous or atypically appearing patterns of groups of DNA sequences (e.g., k-mers and/or reads). In a second stage of supervised learning, the manageable subset of validated anomalous k-mer groups are labeled with biological condition(s) measured in hosts having those k-mer groups, thereby generating training data to train a model to predict the biological condition correlated with those anomalous groups of microbiome DNA sequences.

Step 1 identifies patterns of groups as anomalous or atypical based on a discovery that co-occurrences of k-mers in groups of microbiome DNA sequences follows a power law distribution. Embodiments of the invention may validate a subset of “significant” groups of microbiome DNA sequence that exhibit anomalous co-occurrence patterns that deviate from this power law distribution and so, their expression is unlikely randomly statistical, and therefore likely associated with a phenotypic property associated with a biological condition. The first stage thus eliminates non-validated non-anomalous groups of microbiome DNA sequences, which are likely randomly statistical noise. The training dataset in stage 2 thereby only includes the reduced manageable set of validated anomalous groups of microbiome DNA sequences, making it possible to train the machine learning model based on groups of microbiome DNA sequences in finite practical time. This first stage also reduces the size of the search space from previously unmanageable volumes of all combinations of microbiome DNA sequences groups (e.g., 2^{4\hat{ }30} 30-mer pairs) to a manageable volume of only bundled groups of validated anomalous microbiome DNA sequences (e.g., an order of magnitude of k-mer groups equal to the number of distinct degrees in a network, which depends on how the groups are bundled, and is less than a linear function of the number of individual k-mers in the network). A reduction in training data size from an exponential to a linear function of the number of individual k-mers in the network in stage 1 reduces the storage size and increases training speed by orders of magnitude, thereby improving training efficiency in stage 2.

The first stage may select the anomalous subset of the plurality of microbiome DNA groups as follows. A plurality of microbiome DNA sequences, such as reads and/or k-mers, may be received and stored. In some embodiments, a pre-filtering process may reduce the total number of microbiome DNA sequences to include only repeat sequences (that occur greater than any predefined integer threshold number of times, such as at least twice, in the microbiome DNA) and exclude non-repeat or low-repetition sequences (that occur less than any predefined integer threshold number of times, such as only once or never, in the microbiome DNA). In one example, the filter may reduce the number of k-mers in the model from 4³⁰ total possible 30-mers to an order of one million repeating k-mers. However, even after filtering, the number of groups of microbiome DNA sequences (e.g., >2^{million} k-mer pairs) is too large to train a model in practical time.

Embodiments of the invention solve this problem by reducing the number of the sequence groups intoa computationally manageable number of anomalous groups of microbiome DNA sequences or nodes. This reduction is performed by creating a microbiome network of DNA sequence nodes, then bundling network nodes into potentially or candidate anomalous groups, and then validating potentially anomalous node groups as validated anomalous node groups.

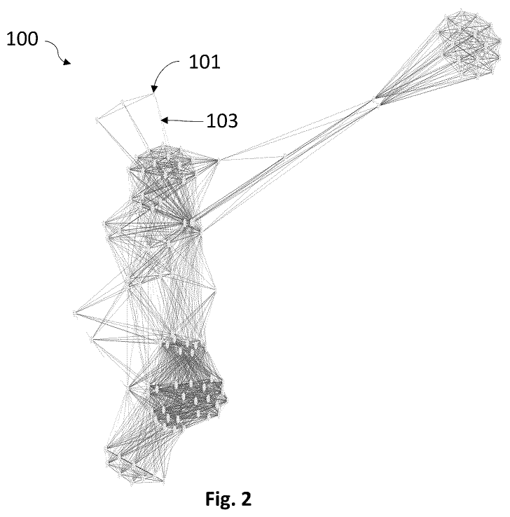

A microbiome network GX=G(V, E_{X}) (e.g., a subregion of network 100 shown in FIG. 2) may be generated comprising a set of a plurality of nodes V={vij} (e.g., nodes 101 of FIG. 2) representing the respective plurality of i unique microbiome DNA sequences (e.g., k-mers and/or reads) and a plurality of edges Ex={vᵢ;vⱼ}(e.g., edges 103 of FIG. 2), each representing a co-occurrence of a unique pair vᵢ and vⱼ of microbiome DNA sequences in a same sub-length or sample of the microbiome DNA (e.g., two k-mers co-occurring in the same read and/or two reads co-occurring in the same DNA sample sequence). In some embodiments, sequences from multiple DNA samples may be combined by super-positioning a plurality of networks each representing a distinct microbiome DNA sample to generate a composite network representing a plurality of microbiome DNA samples (e.g., where different samples are collected from the same animal, farm, herd and/or specie, or from different animals, farms, herds and/or species).

Bundling is performed by sorting nodes in the network into distinct (disjoint or overlapping) groups based on the degree of the group and nodes therein. A node's degree may quantify a number of edges connecting that node (e.g., a number of other unique microbiome DNA sequences that co-occur with that node's microbiome DNA sequence in the same read or DNA sample). Groups may bundle nodes that have the same and/or similar (e.g., +/−an integer degree), and the group's degree may be a dynamic or tunable parameter. This bundles together a group of all sequences (or associated nodes) that co-occur in a DNA sample or sub-length with the same (and/or similar) number of unique other sequences (or associated nodes). A group may bundle DNA sequences that are decentralized (e.g., from different microbes) or centralized (e.g., from the same microbe) in the DNA sample to detect biological effects of decentralized or centralized biomarkers and, additionally or alternatively, may bundle DNA sequences that are contiguous, non-contiguous or a combination of contiguous and non-contiguous in the DNA sample. Microbiome DNA sequence and/or their corresponding nodes may be ordered, e.g., in a sequence, from groups of bundled nodes that are relatively more densely connected (higher degree) to relatively more sparsely connected (lower degree).

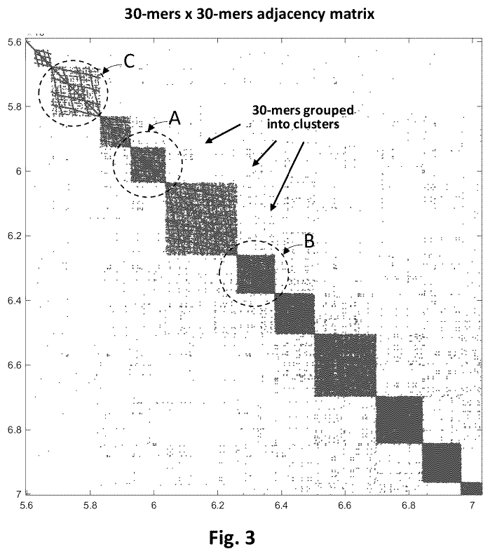

V_{\hat{d}} lidation may then be performed by selecting groups of nodes that have sufficiently deviant (too high or too low) internal connectivity indicating microbiome DNA sequences represented by the nodes co-occur in the same read or DNA sample with sufficiently deviant (high or low) frequency to indicate anomalous expression (e.g., deviating from the expected power law distribution). The internal connectivity of each group of the same degree may be computed, for example, to quantify how inter-connected the sequences or nodes are in the same group. In various embodiments, the internal connectivity may be determined based on a number of nodes in the same group, number of edges in the same group, a number of pre-defined partially or fully connected node clusters (e.g., triangles or polygons or straws or cliques of nodes) in the same group or deviation therefrom. In one embodiment, the internal connectivity may be measured based on a ratio between the number of “internal edges” (edges connecting two nodes of same or similar degree) and the total number of “overall edges” (internal and external edges) for that group of the same degree. In one example, a maximal internal connectivity (e.g., normalized to 1) may indicate the group is a “clique” in which all nodes of that degree are connected to each other internally within the same group and to no other nodes external to the group), and a minimal internal connectivity (e.g., normalized to 0) indicates no internal group connections. Within maximal and minimal limits, a relatively higher internal connectivity indicates a relatively greater interconnection compared to external connection. FIG. 2 visualized the internal connectivity of each of a plurality of groups as the density of nodes in the group's square (internal edges) vs. the total density of nodes in the column and row containing the square (overall edges). For example, groups with relatively high internal connectivity are represented by a relatively highly dense square surrounded by its relatively low density column. A threshold for validating groups as having anomalous inter-connectivity may be based on a discovery that standard groups of sequences or nodes co-occur in DNA reads or samples according to a power law distribution, e.g.: P(degree(v)=d)~d^{-α}, where degree(v) is the number of neighbors node v has in the network, d is a given group degree, and a is a power-law parameter of the network. That is, the standard probability that a sequence or node co-occurs with a number d of different unique other sequences or nodes in the same DNA read or sample diminishes proportionally to a power of that number of co-occurrences. FIGS. 3-6 show this power law relationship. Embodiments of the invention exploit this discovery by validating significant or anomalous groups of sequences or nodes whose internal connectivity or sequence co-occurrence in DNA deviates from (e.g., is significantly greater or less than) an expected internal connectivity in a network that follows a power law distribution, suggesting an abnormal abundance or absence of those groups of sequences or nodes indicating they are anomalous and candidates for correlation with a measured biological condition of the microbiome hosts. In an embodiment (e.g., in theorem 5.8), the internal connectivity condition for a group of nodes having a same degree \hat{d} may be that the group's internal connectivity is greater than or equal to:

$$\frac{{x}_{thres}}{\vert {V}_{\hat{d}} \vert \cdot \hat{d} - {x}_{thres}} ,$$

where xₜₕᵣₑₛ is a threshold in which the probability of observing more than the threshold is less than a value ϵ (e.g., a fixed or tunable confidence parameter) and |V_{\hat{d}}| is the number of nodes in the microbiome network of degree \hat{d}.

In some embodiments, when degrees are too small or too large, groups tend to deviate from an expected neutral node co-occurrence that follows a power law distributions. Accordingly, an additional or alternative pre-validation stage may filter microbiome DNA sequences or groups to include only nodes within a predefined degree range (e.g., ≥dmin and/or ≤dmax, where dmin and dmax are predefined minimum and maximum threshold degrees such as an integer greater than 2, 3, . . . , m), and edges connecting those nodes, thereby eliminating nodes in the network with degree outside that range and the edges connected to those eliminated nodes. Excluding groups with extreme degrees may cause retention of (only) groups that follow the power law distribution to allow more accurate validation of anomalous groups.

After applying the threshold to select the validated anomalous groups of sequences or nodes (e.g., indicating the co-occurrence of the group of nodes is unlikely), these sequences or nodes are used to train the predictive model in the second stage.

In the first stage, bundling network groups reduces the number of microbiome DNA sequences groups to analyze from an unmanageable number of all combinations of microbiome DNA sequences groups (e.g., the order of magnitude of k-mer groups is an exponential of the number of individual k-mers, such as, 2N groups of N k-mer pairs; so even after filtering the k-mers from 4^{k} possible k-mers to approximately 1 million repeating k-mers, the number of groups is 2^{1,000,000} which is still unmanageable) to a manageable number of bundled groups of microbiome DNA sequences (e.g., an order of magnitude of k-mer groups equal to the number of distinct degrees in a network, e.g., which depending on how the groups are bundled is at most a linear or square function of the number of individual k-mers in the network). Consider a microbe network that has a number of nodes |V| and a number of edges |E|. If every node is bundled into one group, based on its degree, then the number of groups will be the number of degrees. The maximal possible degree is |V| (for a node that is connected to every other node) and the minimal possible degree is 1, so the number of different degrees is at most |V| (and probably significantly less). Accordingly, the number of groups would be at most |V|. If every node is bundled into multiple fixed number of groups, based on its degree, then the number of groups is |V| times a fixed number, which is also on the order of the number of nodes O(|V|). All these groupings based on nodes are linear to the number of nodes in the network. If groups are bundled based on edges, similarly, the number of groups would be on the order of the number of edges O(|E|). Because the microbe network has a maximal number of edges that connects every node to every other node, |E|=|V| *|V|, generating a maximal possible number of groups on the order of a square of the number of nodes O(|V|\hat{ }2). The precise group reduction may depend, e.g., on the network architecture (e.g., fully or partially connected, layer structures, how edges are counted such as between all nodes or only adjacent or up to nearby layers, how groups are constructed as only the same or including similar numbers of edges, case-specific network data such as the number or arrangement of nodes, etc.). The precise group reduction may also be a tunable design parameter, such that, degrees are defined to sort nodes into a predefined fixed or dynamic tunable number of groups. Reducing the number of microbiome DNA sequences groups being analyzed from an exponential to (e.g., less than) a linear or square power of the number of individual microbiome DNA sequences converts a previously NP-hard problem solvable in exponential time to a linear problem solvable in linear time. This boon to efficiency makes the previously impractical task of analyzing groups of microbiome DNA sequences computationally practical, and so also makes practical its use for training a predictive model to correlate those groups with biological conditions in hosts, thereby revealing a wealth of new knowledge.

The second supervised learning stage trains a predictive model using a training dataset comprising anomalous microbiome DNA sequence groups (inputs) (or frequency, internal connectivity or scores thereof) correlated to biological conditions in hosts (outputs). The training dataset may comprise only those selected groups validated as anomalous microbiome DNA sequences (e.g., a minority, such as, a relatively small number of groups, such as hundreds of thousands or millions, compared to 2^{1,000,000} possible groups) and excluding the remaining (e.g., vast majority of) non-validated groups of microbiome DNA sequences that either failed validation or were not bundled as candidates for validation. Generating the training dataset with (only) validated anomalous groups of microbiome DNA sequences (excluding non-validated non-anomalous groups of microbiome DNA sequences) also significantly reduces the storage size of the training dataset (e.g., by a reduction factor of #kmers/2^{#kmers}, such as, 10 6/2^{1,000,000} or more) compared to a training dataset containing all sequence groups and thereby increases training speed and efficiency (e.g., by the same reduction factor) based on that training dataset to terminate in practical time. The predictive model may be trained by any machine learning model including, but not limited to, neural networks or deep learning, linear regression, logistic regression, support vector machines, ensemble learning, decision forests, K-mean clustering, and/or K-nearest neighbor algorithms.

In some embodiments, stages 1 and 2 of the training phase may be repeated to retrain the model based on new microbiome DNA (e.g., for a periodic collection, new herds, etc.). In some embodiments, stages 1 and 2 may or may not be repeated at the same rate. For example, stage 1 may only be executed once or periodically and not for each model retraining (e.g., when stage 1 is executed globally based on at least some hosts located non-local to a farm or area for which prediction is sought), whereas stage 2 may be executed for each model retraining (e.g., executed locally based (only) on hosts located local to the farm or area for which prediction is sought).

After the model is trained in training phase, a predictive run-time phase may be executed in which the model inputs new microbiome DNA sequences from one or more hosts and outputs a prediction of the biological condition for one or more same or different hosts. Steps may be taken to change or preemptively avoid or encourage the predicted biological condition for one or more same or different hosts than the hosts for which the biological condition is predicted. For example, a first group of hosts (e.g., to which feed, additive or medicine was administered) having measured biological conditions (e.g., positive health benefits or reduced health detriments) may be used to train the model; a second group of hosts (e.g., to which no feed, additive or medicine was administered) may be predicted to have those biological conditions (e.g., positive health benefits or reduced health detriments); which may trigger a practice to alter or maintain the biological condition of a third group (e.g., to administer that feed, additive or medicine to), where all three groups are distinct (disjoint or overlapping), but for example, are associated by living in a neighboring herd or region, from the same family or ancestry or have similar genetic profiles.

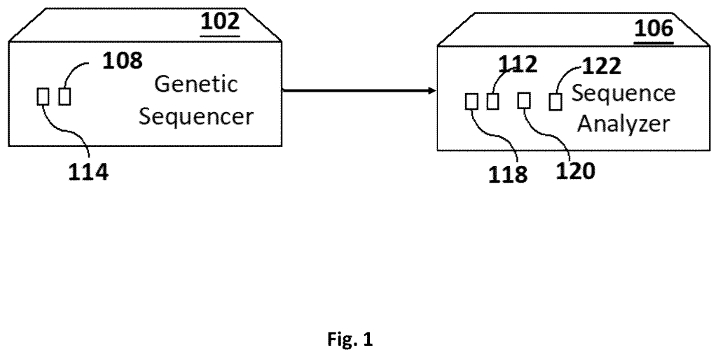

Reference is made to FIG. 1, which schematically illustrates a system for accurately and efficiently detecting biomarkers that are groups of DNA sequences in microbiome DNA of one or more hosts to predict a biological condition, according to an embodiment of the invention.

The system of FIG. 1 may include a genetic sequencer 102 and/or a sequence analyzer 106. Units 102 and 106 may be implemented in one or more computerized devices as hardware and/or software units, for example, specifying instructions configured to be executed by one or more processors. One or more of units 102 and 106 may be implemented as separate devices or combined as an integrated device.

Genetic sequencer 102 may input DNA obtained from biological samples, such as, a microbiome sample of one or more real living host organisms and may output each organism's genetic sequence including the host's genetic information at one or more genetic loci, for example, a plurality of microbiome DNA sequences. In some embodiments, genetic sequencer 102 may input a biological sample, perform shotgun fragmentation (e.g., randomly breaking up the genome into small DNA fragments), sequence each fragment individually, order the sequenced fragments by running a computer program to detect overlaps in the DNA sequences, and reassemble the fragments in their correct order to reconstitute the DNA genome. Each single organism's DNA sample may be sequenced for analysis as an individual set of microbiome DNA sequences or multiple organism's DNA samples may be sequenced for analysis as a combined set of the host group's microbiome DNA sequences.

Genetic sequencer 102 may store in one or more memorie(s) 114, and send to sequence analyzer 106 to receive and store in one or more memorie(s) 118, a plurality of microbiome DNA sequences (e.g., reads and/or k-mers), sequenced from microbiome DNA of one or more hosts, for example, each microbiome DNA sequence having a fixed or variable length of nucleotides (e.g., 100-150 nucleotides in each read and/or k nucleotides in each k-mer, such as, k=30-60). To reduce the volume of the microbiome DNA sequences for analysis, e.g., prior to generating a microbiome network, genetic sequencer 102 and/or sequence analyzer 106 may filter the plurality of microbiome DNA sequences to include (only) microbiome DNA sequences that occur greater than a predefined integer number of times in the same sample or sub-length of the microbiome DNA, thereby including (only) sufficiently repeating microbiome DNA sequences and excluding non-repeating or insufficiently repeating microbiome DNA sequences.

Sequence analyzer 106 may generate a microbiome network (e.g., 100 of FIG. 2) comprising a plurality of nodes (e.g., 101 of FIG. 2) representing the respective plurality of microbiome DNA sequences and a plurality of edges (e.g., 103 of FIG. 2) connecting the nodes representing a co-occurrence of each pair of microbiome DNA sequences in a same sample or sub-length of the microbiome DNA (e.g., pairs of reads in a string of the entire or partial length of DNA or pairs of k-mers in the same read). Each host's DNA sample may be represented by a network of sequence nodes, so combining multiple hosts DNA sequences may be performed by merging or super-positioning a plurality of host-specific to generate a composite network representing a plurality of microbiome DNA samples.

Sequence analyzer 106 may then bundle microbiome DNA sequences represented by nodes into distinct (e.g., disjoint or overlapping) candidate or potential anomalous groups, based on the node degrees. The degree of each node may quantify a number (e.g., or density, deviation from a mean or median thereof, etc.) of edges that contain the node indicating a number (e.g., or density, deviation from a mean or median thereof, etc.) of unique microbiome DNA sequences that co-occur with the microbiome DNA sequence represented by the node in the same sample or sub-length of the microbiome DNA. The degree condition for bunding nodes in the same group may be that the nodes have the same or similar degree, such as, +/−some integer or percentage deviation from the node's degree. The degree condition for bunding nodes may be a tunable or dynamic parameter, e.g., manually set by a user or automatically set to optimize computer efficiency.

Sequence analyzer 106 may then validate candidate or potential anomalous groups into confirmed anomalous groups by measuring each group's internal connectivity and selecting or validating groups of microbiome DNA sequences that each have an internal connectivity between nodes of the same group that satisfies an anomaly condition indicating the microbiome DNA sequences of that group co-occur with a probability that is unlikely randomly statistical. In some embodiments, the internal connectivity of each group may be determined based on a number of nodes in the same group, a number of edges in the same group, a number of pre-defined partially or fully connected clusters (e.g., triangles or polygons or straws (linearly connected) or cliques) in the same group or deviation thereof. Additionally or alternatively, the internal connectivity of each group may be determined based on a ratio between a number of internal edges connecting two nodes internal to the same group (e.g., visualized by the number or density of points inside each distinct box in the graph of FIG. 3) and a total number of overall edges connecting nodes within the group to any other node internal or external to the group (e.g., visualized by the number or density of points in rows and columns intersecting the group's box in the graph of FIG. 3). In some embodiments, the anomaly condition is that the internal connectivity for the group of nodes deviates from an expected internal connectivity in a network that follows a power law distribution. Such a deviation from a power law deviation establishes a condition for the internal connectivity of the ratio above for a group of nodes having a same degree \hat{d}, that the group's internal connectivity is greater than or equal to:

$$\frac{{x}_{\text{?}}}{\vert {V}_{\text{?}} \vert \cdot \hat{d} - {x}_{thres}} ,$$

$$\text{?} \text{indicates text missing or illegible when filed}$$

where xₜₕᵣₑₛ is a threshold in which the probability of observing more than the threshold is less than a value ϵ (e.g., a fixed or tunable confidence parameter) and |V_{\hat{d}}| is the number of nodes in the microbiome network of degree \hat{d}.

The combination of bunding and validating together provides an efficient and accurate technique for selecting anomalous groups of microbiome DNA sequences, and excluding non-anomalous groups of microbiome DNA sequences, to model those groups' correlation with biological condition more accurately and efficiently. Without bundling, the number of combinations of groups to validate for anomaly conditions is on the order of an exponential of the number of groups (e.g., 2^{#nodes}). Bundling nodes into disjoint groups reduces the number of combinations of groups to validate for anomaly conditions to, for example, a linear order of the number of groups (e.g., O(#nodes)) or at most a square of the number of nodes (e.g., O(#nodes²), depending on the grouping). In some embodiments, more or less compact groupings may be used depending on network size, computer resources, convergence times, etc. For example, group numbers with a linear order of nodes may be used for larger networks, while group numbers with a square order of nodes may be used for relatively smaller networks. This reduction makes a realistically impossible NP-hard (exponential) problem (e.g., analyzing O(2^{#nodes}) groups) to a realistically possible problem (e.g., analyzing O(#nodes) and/or ≤O(#nodes²) groups).

In some embodiment, groups with extreme degrees (greater than and/or less than predefined integer threshold degree(s), such as, outside 90% of the standard deviation from the mean degree) may deviate from an expected neutral node co-occurrence that follows a power law distributions used to validate groups to train the model. In one embodiment, prior to bundling and/or validation, sequence analyzer 106 may filter the plurality of microbiome DNA sequences to include (only) nodes with degrees within a predefined range and edges connecting those nodes, eliminating nodes deviating from the predefined degree range and edges connected to the eliminated nodes. In another embodiment, other neutral distributions and thus, conditions, may for the basis for validating groups of these extreme degrees.

After validation, sequence analyzer 106 may generate a training dataset correlating the validated groups of microbiome DNA sequences from one or more first hosts with a biological condition measured in one or more of same or different second hosts (e.g., different hosts in the same herd, family and/or having a similar genetic profile).

Sequence analyzer 106 may then train a machine learning model with the training dataset to input microbiome DNA sequences from one or more third hosts and predict the biological condition for one or more of same or different fourth hosts (e.g., different hosts in the same herd, family or having a similar genetic profile). Training may be repeated, e.g., periodically (such as, seasonally), based on new microbiome DNA.

Sequence analyzer 106 may then run a prediction phase in which the model inputs new microbiome DNA sequences from one or more fifth hosts and outputs a prediction of the biological condition for one or more same or different sixth hosts. Steps may be taken to change or preemptively avoid or encourage the predicted biological condition for one or more same or different hosts than the hosts for which the biological condition is predicted.

Genetic sequencer 102 and sequence analyzer 106 may include one or more controller(s) or processor(s) 108 and 112, respectively, configured for executing operations and one or more memory unit(s) 114 and 118, respectively, configured for storing data such as genetic information or sequences and/or instructions (e.g., software) executable by a processor, for example for carrying out methods as disclosed herein. Processor(s) 108 and 112 may include, for example, a central processing unit (CPU), a digital signal processor (DSP), a microprocessor, a controller, a chip, a microchip, an integrated circuit (IC), or any other suitable multi-purpose or specific processor or controller. Processor(s) 108 and 112 may individually or collectively be configured to carry out embodiments of a method according to the present invention by for example executing software or code. Memory unit(s) 114 and 118 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units. Genetic sequencer 102 and sequence analyzer 106 may include one or more input/output devices, such as output display 120 (e.g., such as a monitor or screen) for displaying to users results provided by sequence analyzer 106 (e.g., visualizing FIGS. 2-9) and an input device 122 (e.g., such as a mouse, keyboard or touchscreen) for example to control the operations of the system and/or provide user input or feedback, such as, selecting one or more hosts, selecting input genetic sequences, selecting one or more tuning parameters for model training, inputting new training data and retraining, selecting biological conditions (e.g., from a predefined set) to train or predict, selecting training accuracy, iterations or times, etc.

Reference is made to FIG. 2, which schematically illustrates data structures representing a portion of a microbiome network 100 of microbiome DNA sequences, according to an embodiment of the invention. Microbiome network 100 typically has too many nodes, for example, millions, to illustrate in its entirety. Data structures described herein may be stored in one or more memor(ies) (e.g., memory unit(s) 114 and/or 118 of FIG. 1), may be generated and controlled by one or more processor(s) (e.g., controller(s) or processor(s) 112 of a DNA sequence analyzer 106 of FIG. 1), and any visualizations, or data thereof may be displayed on one or more display(s) (e.g., output display 120 of FIG. 1 or any user display).

Microbe network 100 comprises a plurality of nodes 101 representing a respective plurality of microbiome DNA sequences, each sequenced from a biological sample of one or more hosts. The plurality of nodes 101 may be pairwise connected by a plurality of edges 103. Each edge 103 may represent a co-occurrence of each pair of microbiome DNA sequences in a same sample or sub-length of the microbiome DNA, such as, two reads in the same partial or whole string of DNA or two k-mers in the same read. Nodes 101 may be densely-connected by edges 103 (e.g., >80% of nodes connected by edges), sparsely-connected by edges 103 (e.g., <20% of nodes connected by edges) or moderately-connected by edges 103 (e.g., 20-80% of nodes connected by edges). Nodes 101 and/or edges may be filtered, e.g., to remove edges having extreme degree (e.g., ≥dmin and/or ≤dmax, where dmin and dmax are predefined minimum and maximum threshold degrees).

Nodes 101 may each have a degree counting the number of edges to which the node connects. Nodes 101 may be bundled into groups of the same or similar degree. Nodes 101 in the same group thus have the same or similar number of unique microbiome DNA sequences that co-occur with the microbiome DNA sequence represented by the node in the same sample or sub-length of the microbiome DNA. Each bundled group may be validated to determine if it has sufficiently anomalous behavior to correlate with host biological condition for training a machine learning model, for example, if the nodes 101 of the group are inter-connected with a probability that is unlikely randomly statistical (e.g., too high or too low, indicating the nodes' co-occurrence is likewise unlikely randomly statistical, but significant, and therefore likely correlated with host biological condition). The anomaly condition may be, for example, that the internal connectivity for the group of nodes deviates from an expected internal connectivity in a network, such as, one that follows a power law distribution. Internal connectivity of nodes 101 by edges 103 may be a measure of a number of nodes 101 or edges 103 in the same group, a density of nodes 101 or edges 103 in the network 100 or region thereof, a number of pre-defined partially or fully connected node clusters (e.g., triangles or polygons or straws or cliques of nodes) in the same group, a ratio of intra-group edges (internal to a group) to inter-group edges or overall edges (external to a group or in the entire network or region thereof), or derivative or deviation therefrom. Groups of microbiome DNA sequences represented by nodes 101 in validated anomalous groups may be used, together with biological conditions measured for their DNA's hosts, to generate a training dataset to train a machine learning model to corelate groups of microbiome DNA sequences with their host(s)' biological condition(s).

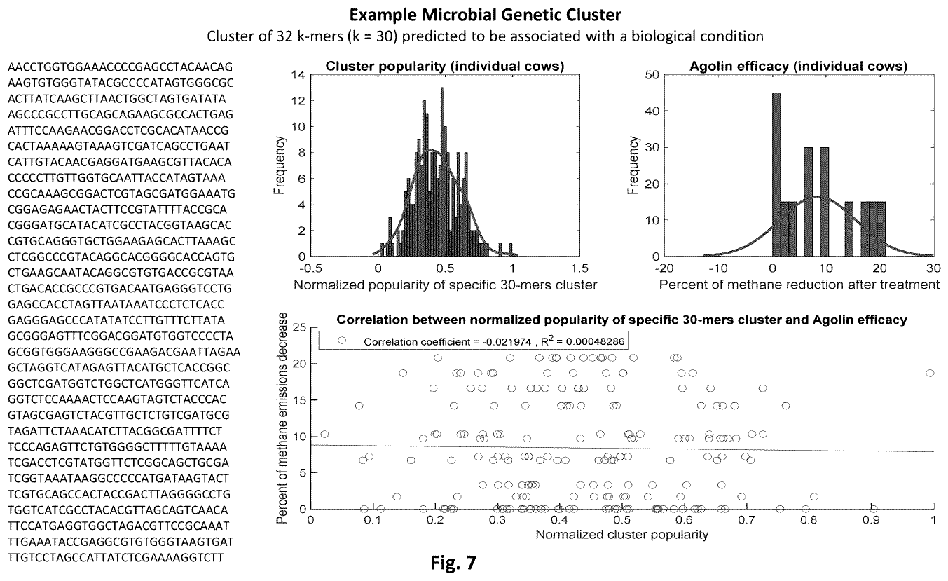

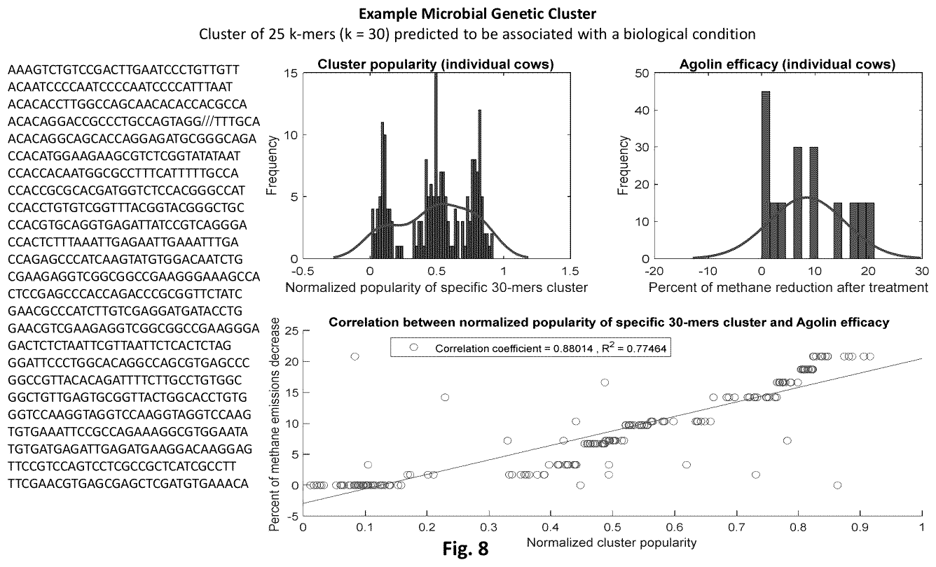

Reference is made to FIG. 3, which is a graph of an adjacency matrix of a network of microbiome DNA sequences clustered into groups, according to an embodiment of the invention. Each point in the groups represents a node. FIG. 3 may visualize the network of microbiome DNA sequences represented by an adjacency matrix. The adjacency matrix may be an N*N matrix, such that each coordinate (n1,n2) is binary (e.g., either 0 or 1), representing whether or not the graph contains an edge between node n1 and n2. The order to the nodes of the graph may be unordered, so that, the nodes can be sorted in many different ways—each associated with a different adjacency matrix (e.g., differing in the ordering of the nodes, but having the same number of edges, degree distribution and so on, since all these matrices still represent the same graph). One example way to sort the nodes is: (1) sort the nodes by their degree, and then (2) group the nodes based on similar degree values. FIG. 3 shows that when nodes are grouped by degree, the internal connectivity of groups is easily visualized. For example, relatively “dense” group clusters emerge (e.g., containing groups with a relatively high number of internal edges), such as, the square labeled by circles “A” and “B” (analyzed in reference to FIGS. 7 and 8), and relatively “sparse” group clusters emerge (e.g., containing groups with a relatively low number of internal edges), such as, the square labeled by circle “C”. In one embodiment, FIG. 3 visualizes an internal connectivity of each group as a ratio between the number or density of points inside each distinct box (e.g., a number of internal edges connecting two nodes internal to the same group) and the number or density of points in the rows and columns intersecting the group's box (e.g., a total number of overall edges connecting nodes within the group to any other node internal or external to the group).

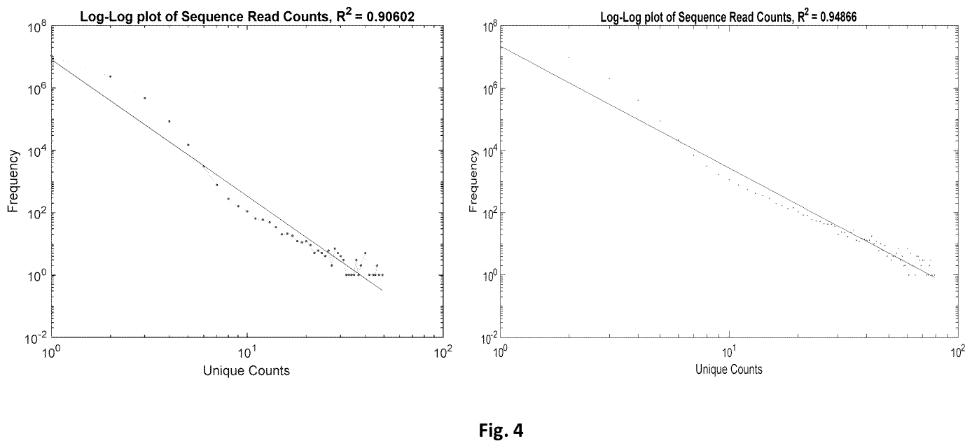

Reference is made to FIG. 4, which are two graphs of the relationship between the number of reads (y-axis) and the number of times those reads repeat (x-axis), according to an embodiment of the invention. For example, there are millions of reads that appear one time, and about a hundred reads that repeat 10 times, and very few reads that repeat dozens of times (left chart). The left chart represents reads in a single sample, and the right chart represents reads from many combined samples. Both graphs show a power-law relationship between the number of reads (y-axis) and the number of times those reads repeat (x-axis) (e.g., the data approximately, on average, following a linear relationship in a double-log scale). Both graphs represent microbiome DNA collected from cow data.

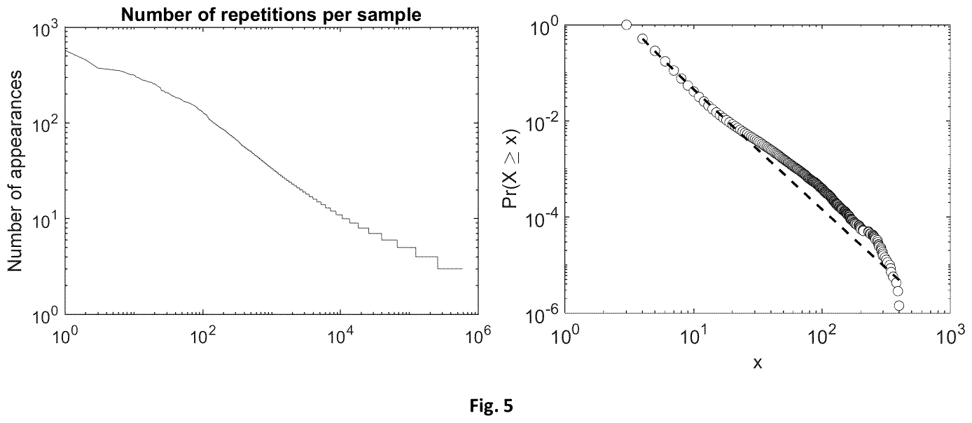

Reference is made to FIG. 5, which includes two graphs representing distributions of repetitions of DNA reads combining sequences sequenced from multiple biological samples from multiple hosts, according to an embodiment of the invention. The left graph may represent the relationship between the number of reads (x-axis) and the number of times those reads repeat (y-axis) in the combined DNA sequences of the multiple hosts. The right graph may represent the relationship between the probability (y-axis) that the number of repetitions for a given node is greater than or equal to x (x-axis) in the combined DNA sequences of the multiple hosts. The probability in the right graph decreases monotonically (e.g., since every nodes that repeat X+1 times also repeats, by definition, X times), and there is some value for which it receives 1 (e.g., the minimal number of repetitions for the nodes in the network). Both graphs follow a power law distribution. Both graphs represent microbiome DNA from the human microbiome project.

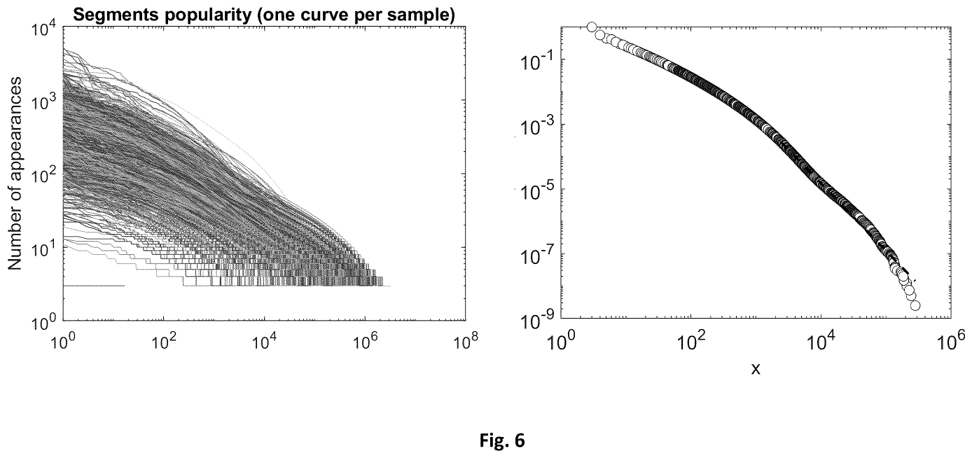

Reference is made to FIG. 6, which includes two graphs representing distributions of repetitions of reads sequenced from the DNA of a single host obtained from a distinct biological sample, according to an embodiment of the invention. In the left graph, each curve represents, for the DNA of a single distinct host obtained from a distinct biological sample, the relationship between the number of reads (x-axis) and the number of times those reads repeat (y-axis). The right graph may represent the relationship between the probability (y-axis) that the number of repetitions for a given node is greater than or equal to x (x-axis).

Whereas FIG. 5 shows a power law distribution for a DNA sequence of microbiomes from multiple samples combined together, FIG. 6 shows approximately the same power-law distribution for (almost) every single host's sample individually (individual curve in the right graph of FIG. 6).

Reference is made to FIG. 7, which is a graph of an analysis of a clustered group of microbiome DNA sequences labeled by circle “A” in FIG. 3 (e.g., generated by bundling and validated with an anomaly criterion), according to an embodiment of the invention. FIG. 7 shows four elements:
1. A list of k-mers in the clustered group labeled by circle “A” in FIG. 3.
2. A distribution of the total appearance of these k-mers among the tested hosts (e.g., approximating a statistically neutral Gaussian distribution).
3. A distribution of the efficacy of the additive among the tested hosts (e.g., the biological condition the model is trained to predict). This example data was collected from farms from which cows were taken, calculated based on the cows whose methane was measured, which are different cows than the tested cows (e.g., the microbiome cows).
4. Correlation between (2) distribution of the total appearance of these k-mers among the tested hosts and (3) distribution of the efficacy of the additive among the tested hosts. In the example of the group labeled by circle “A,” there is no significant correlation.

Reference is made to FIG. 8, which is a graph of an analysis of a clustered group of microbiome DNA sequences labeled by circle “B” in FIG. 3 (e.g., generated by bundling and validated with an anomaly criterion), according to an embodiment of the invention. FIG. 8 shows four elements:
1. A list of k-mers in the clustered group labeled by circle “B” in FIG. 3.
2. A distribution of the total appearance of these k-mers among the tested hosts (e.g., deviating from a statistically neutral Gaussian distribution).
3. A distribution of the efficacy of the additive among the tested hosts (e.g., the biological condition the model is trained to predict). This example data was collected from farms from which cows were taken, calculated based on the cows whose methane was measured, which are different cows than the tested cows (e.g., the microbiome cows).
4. Correlation between (2) distribution of the total appearance of these k-mers among the tested hosts and (3) distribution of the efficacy of the additive among the tested hosts. In the example of the group labeled by circle “B,” there is a significant correlation between these k-mers and the biological condition (e.g., efficacy of the feed additive). This group can thus be used for prediction.

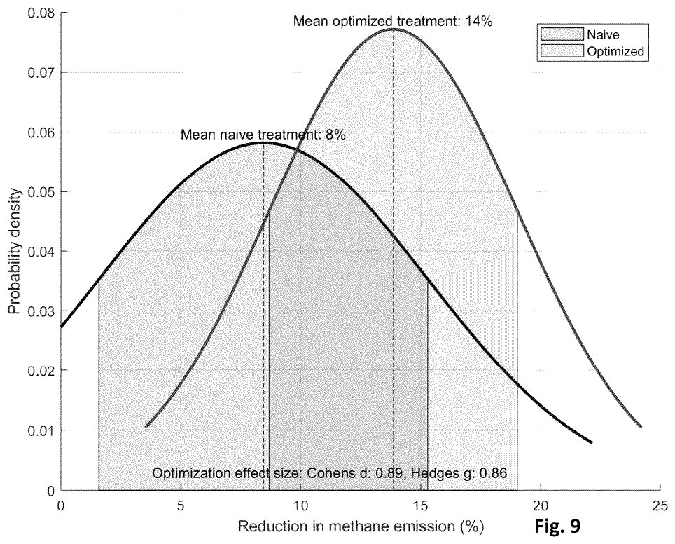

Reference is made to FIG. 9, which is a graph of two distributions related to biological condition, according to an embodiment of the invention. The two distributions each show a distribution of the efficacy of the Agolin additive (y-axis) vs. percent of CH₄ emissions reduction (x-axis) measured at farms. Efficacy was calculated as detailed herein (based on a change in emissions from prior to testing to after testing, normalized by a change of a control group that received no additive). The right distribution is a distribution for an entire population (e.g., 13 farms) and the left distribution is a distribution for approximately 50% of the population (e.g., 6 farms) for which the machine learning model predicted would have the highest efficacy. The efficacy average of the right distribution (optimal sub-population) is shown to be significantly greater than that of the left distribution (entire population), indicating the machine learning model may be used to successfully select hosts for administering the Agolin additive (e.g., administered to cow herds on the 8 farms predicted to experience the highest Agolin efficacy). Efficacy was measured “out of sample”, i.e., using different microbiome cows, and validated with different methane cows, compared to the microbiome and methane cows used for the training.

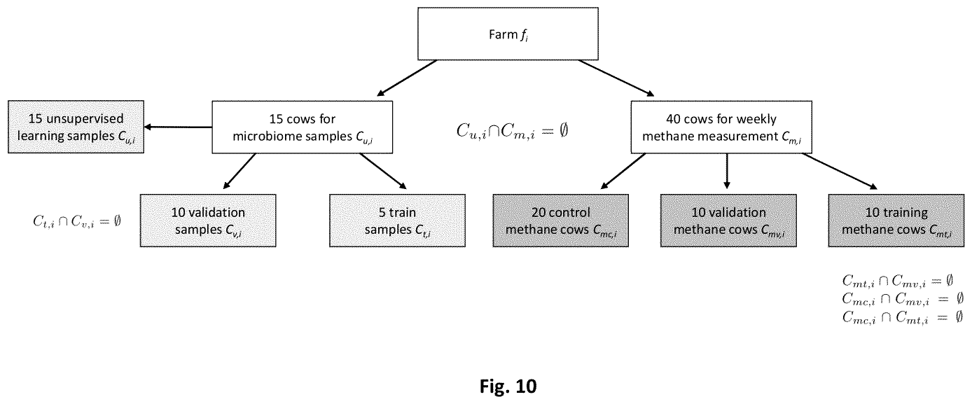

Reference is made to FIG. 10, which schematically illustrates field test data structures, according to an embodiment of the invention. The field test was conducted on a farm fi testing a predefined number of (e.g., 15) cows for microbiome samples C_{u,i} to generate the predefined number of (e.g., 15) respective unsupervised learning samples C_{u,i} comprising 10 validation samples C_{v,i} and a predefined number of (e.g., 5) training samples C_{t,i} that are distinct (C_{v,i}∩C_{t,i}=Ø). The field test tested a predefined number of (e.g., 40) cows from farm fi for weekly methane measurement C_{m,i} comprising a predefined number of (e.g., 20) control methane cows C_{mc,i}, a predefined number of (e.g., 10) validation methane cows C_{mv,i}, and a predefined number of (e.g., 10) training methane cows C_{mt,i}, of all distinct cows (C_{me,i}∩C_{mv,i}=Ø; C_{me,i}∩C_{mt,i}=Ø; C_{mt,i}∩C_{mv,i}=Ø). The following example field test methodology was used: (a) microbiome sampling cows used in each farm are completely different than methane test cows (C_{u,i}∩C_{m,i}=0) (e.g., to ensure predictions apply on a herd level, and are not effects specific to the microbiome of individual cows); (b) training cows are different than validation cows, both for microbiome and for methane (e.g., to provide out-of-sample prediction rigor); and (c) the same control cows were used for the training and validation to ensure uniform normalization of efficacy, not biased by the identity of control cows).

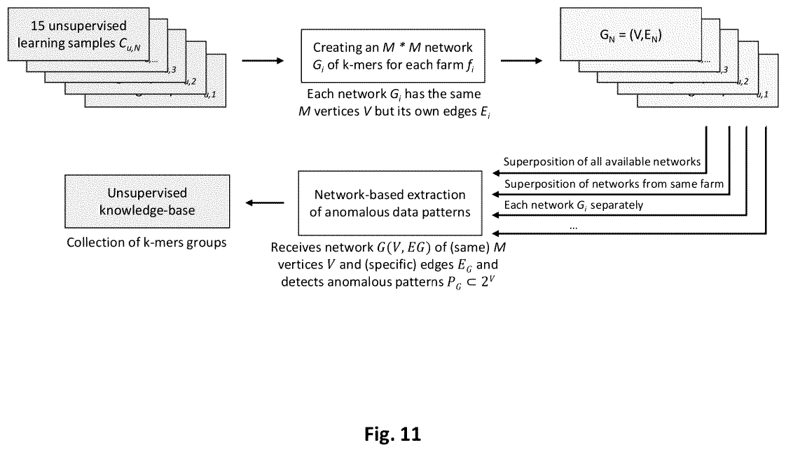

Reference is made to FIG. 11, which schematically illustrates data structures of an example first unsupervised learning stage of the two-stage technique for detecting biomarkers that are groups of microbiome DNA sequences, according to an embodiment of the invention. From each farm fi, a predefined number of (e.g., 15) microbiome cows may be sampled, and a microbiome network of k-mers may be generated. From each network, groups of k-mers are extracted or bundled, and anomalous groups are validated based on an anomaly criterion (non-anomalous groups are not validated). In some embodiment, multiple networks may be combined as a superposition of single-farm networks (e.g., representing all networks or a sub-set of networks, such as, network from the same farm, herd, etc.). All or a filtered subset of validated groups may then be used for the second supervised learning stage of the two-stage technique supervised phase.

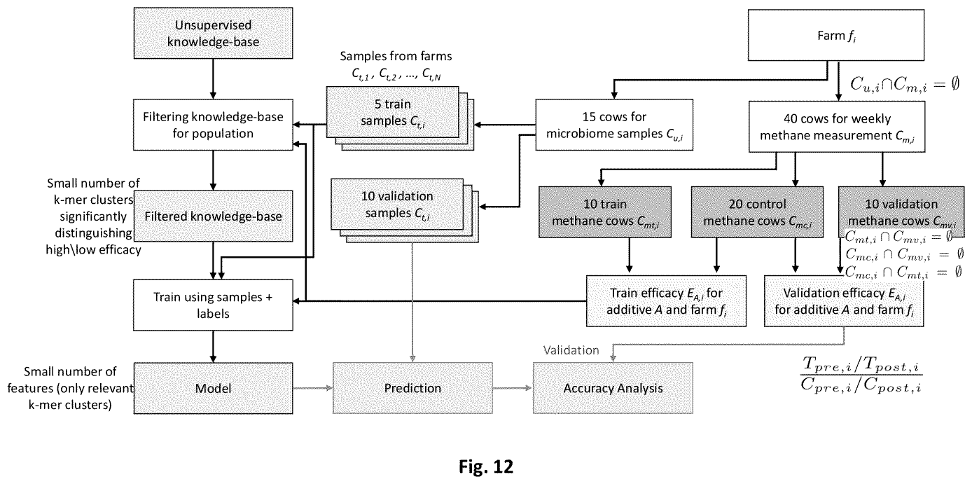

Reference is made to FIG. 12, which schematically illustrates data structures of an example second supervised learning stage of the two-stage technique for detecting biomarkers that are groups of microbiome DNA sequences, according to an embodiment of the invention. A field test may be used, e.g., as described in reference to FIG. 10. A predefined number of (e.g., 10) methane cows may be selected from each farm and their emissions measured before the trial and after the trial, to calculate their change in emissions. This change may be normalized by a similar change of the (e.g., 20) control cows, which received no treatment. These emission measurement may be the labels provided for training. Then, a predefined number (e.g., 10) of different methane cows may be measured and normalized. These emission measurement may be the labels the model is trying to predict. The training phase also uses a predefined number of (e.g., 5) microbiome cows from each farm as input for training. The “features” for training may be the groups identified in the first unsupervised learning stage, and in this second stage it may be determined whether each of them has a correlation or no correlation with the labels. The result is a machine learning model that identities microbiome sequence groups with correlation to biological condition labels. The model may input the combination of multiple microbiome sequences, for example, aggregated as a weighted function. For example, the weighted function may aggregate (e.g., 20 different) k-mers that are relevant to a biological condition based on their occurrence in the sample, their frequency, a subset of the most frequently or infrequently occurring k-mers, etc. The performance of the machine learning model may then be tested using a predefined number of (e.g., 10) microbiome cows from each farm and labels from a predefined number of (e.g., 10) validation methane cows.

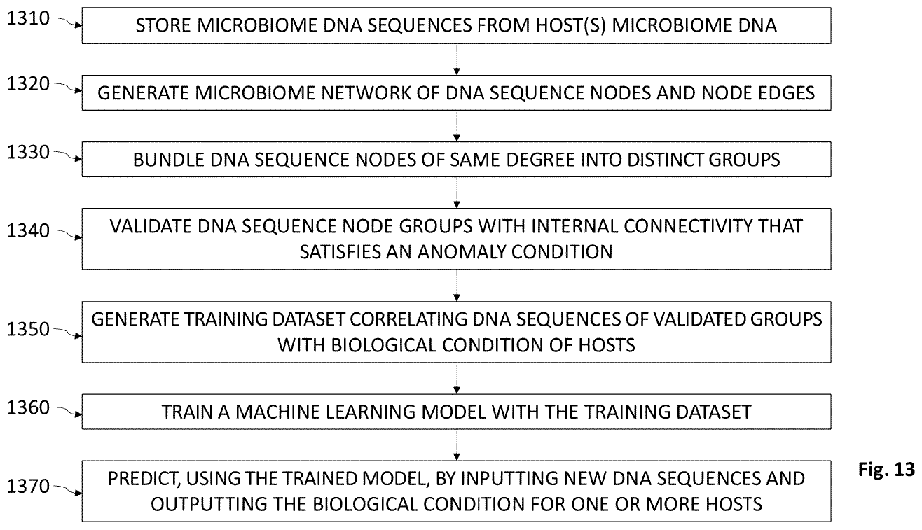

Reference is made to FIG. 13, which is a flowchart of a method for accurately and efficiently detecting biomarkers that are groups of DNA sequences in microbiome DNA of one or more hosts to predict a biological condition, according to an embodiment of the invention. Operations described herein may be executed by one or more processor(s) (e.g., controller(s) or processor(s) 112 of a DNA sequence analyzer 106 of FIG. 1), data or data structures described herein may be stored in one or more memor(ies) (e.g., memory unit(s) 118 of a DNA sequence analyzer 106 of FIG. 1), and any visualizations, or data may be displayed on one or more display(s) (e.g., output display 120 of FIG. 1 or any user display).

In operation 1310, a process or processor may store a plurality of microbiome DNA sequences (e.g., DNA reads or k-mers) that are sequenced from microbiome DNA of one or more livestock hosts.

In operation 1320, a process or processor may generate a microbiome network comprising a plurality of nodes representing the respective plurality of microbiome DNA sequences and a plurality of edges representing a co-occurrence of each pair of microbiome DNA sequences in a same sample or sub-length of the microbiome DNA (e.g., reads in string or k-mers in reads).

In operation 1330, a process or processor may bundle, into distinct (e.g., disjoint or overlapping) groups (e.g., candidate or potential anomalous groups), microbiome DNA sequences that are represented by nodes based on the node's degree. The degree of each node may quantify a number of edges that contain the node indicating a number of unique microbiome DNA sequences that co-occur with the microbiome DNA sequence represented by the node in the same sample or sub-length of the microbiome DNA.

In operation 1340, a process or processor may validate groups of microbiome DNA sequences as anomalous that each have an internal connectivity between nodes of the same group that satisfies an anomaly condition indicating the microbiome DNA sequences of that group co-occur with a probability that is unlikely randomly statistical. The process or processor may determine that the internal connectivity condition is satisfied if the internal connectivity for a group of nodes deviates from an expected internal connectivity in a network that follows a power law distribution.

In operation 1350, a process or processor may generate a training dataset correlating the validated anomalous groups of microbiome DNA sequences from one or more livestock hosts with a biological condition measured in one or more same or different livestock hosts.

In operation 1360, a process or processor may execute a training phase to train a machine learning model with the training dataset to input microbiome DNA sequences from one or more livestock hosts and predict the biological condition for one or more same or different livestock hosts.

In operation 1370, a process or processor may execute a predictive run-time phase, in which the model inputs new microbiome DNA sequences from one or more hosts and outputs a prediction of the biological condition for one or more same or different hosts.

Other or different operations or orders of operations may be used and some operations may be omitted repeated, e.g., operations 1310-1360 may be repeated (e.g., periodically, upon receiving new data, etc.) to retrain the model with new microbiome DNA sequences stored in operation 1310 from a new host biological sample.

Some embodiments of the invention adopt an unbiased metagenomic approach to create a model that determines the most suitable feed additive customized for individual herds, allowing for precision application based on individual microbiome profiles. The technique disclosed herein acknowledges the significant variation and multitude of contributing factors that lead to the diverse responses observed among ruminants. Such embodiments exploit the rich biological information stored in the rumen microbiome, for example, transforming the microbiome of a select few hosts (e.g., cows in each herd) into a living ‘sensor’, allowing for the prediction of biological effect, such as, reduced maximal methane emission to determine the most effective feed additive tailored to that specific herd.

Some embodiments use a two-stage trial design targeting the prediction of the biological efficacy, e.g., of methane-reducing additives, using cows' microbiome data. The first stage may engage an unsupervised machine learning process, trained on a diverse dataset, that for example includes microbiome samples from a wide spectrum of cows across various farms. The second stage may use a smaller subset of cows, for example, whose methane emissions have been documented periodically, to implement supervised learning. The second stage may construct a predictive model that associates microbiome profiles with the effectiveness of feed additives.

By directly tapping into the microbiome, embodiments of the invention may bypass conventional variables such as weather and diet, which, though traditionally deemed critical, pose a challenge in establishing a clear, direct association with biological effect, such as, feed additive efficacy. This provides an objective and comprehensive solution designed to effectively mitigate methane emissions in livestock farming.

Benefits include, not only in the use of the microbiome as a predictive tool, but also in the capacity to make sense of its complex raw data. The rumen microbiome, rich in diversity and complexity, conventionally presents a significant analytical challenge that until now has hindered its utility in such applications. To tackle this, a data-driven approach may be used powered by state-of-the-art artificial intelligence technology, creating an intelligent model that acknowledges the extensive variation and plethora of factors contributing to diverse responses observed among ruminants. By leveraging the power of the microbiome and artificial intelligence, embodiments of the invention provide an accurate and effective solution for environmentally-conscious livestock management.

This technique may be scalability, applicable beyond methane reduction to predict any measurable biological condition, and provides potential for continuous predictive improvement as more data accumulates. Some embodiments of the invention provide prospective influence on environmental sustainability, farm economy, and precision agriculture.

Experiments were conducted in which an extensive dataset encompassing hundreds of rumen microbiome samples were gathered and sequenced using deep metagenomic shotgun sequencing, yielding an average of 14 million reads per sample. Additionally, thousands of efficacy readings were acquired for several leading commercial feed additives, tested across a diverse cohort of dairy cattle in 22 different trial sites. To develop a predictive model, experiments employed novel artificial intelligence techniques according to embodiments of the invention that leverage the microbiome's genetic data to estimate the efficacy of these feed additives across the different farms. The robustness of this predictive model was affirmed through validation with independent cohorts, ensuring its generalizability and reliability.

These findings highlight the transformative potential of employing targeted feed additive strategies to maximize dairy yields while simultaneously reducing methane emissions. Specifically, experiments show that the predictive model generated according to embodiments of the invention could more than double the average efficacy of the tested additives by assigning each to the farms where they would have the highest impact, reaching an average potential reduction of 30% in overall emissions. Experiments show a substantial stride towards sustainable dairy farming, synergistically leveraging advanced data-driven approaches and microbiome science to refine livestock nutritional strategies. By harnessing the power of the rumen microbiome, embodiments of the invention aim to guide the trajectory towards a future of productive dairy farming that is also environmentally conscious.

### Microbiome Acquisition And Ruminal Fluid Sampling

Ruminal fluid samples were collected using an oral stomach tube (ST). For the ST a 300-cm long polyvinyl chloride orogastric tubing (2.9 cm O.D. and 2.5 cm I.D.) was used with 4 holes perforated at the last 30 cm of the ST on the stomach side end. During a sampling event, the head of the animal was restrained. Then, the oral end of the ST was inserted to a 50 cm long speculum with a fixation wire on its end. The speculum with the first part of the ST was inserted into the oral cavity of the animal and was fixated with its wire around the snout. Thereafter, the ST was gently inserted through the esophagus into the rumen while approximately 100 cm of the ST was left outside the animal. This part of the ST was kept lower than the animals head until ruminal fluid was observed passively accumulating at the lowest part of the ST. Approximately 10 ml of initially sampled ruminal fluid was discarded due to possible saliva contamination. After discarding the initial volume, an additional 50 ml of ruminal fluid were collected to a 50 ml sterile conical tube and processed for further analyses. Between each animal sampling the ST and the speculum was thoroughly rinsed and bleached to avoid cross contamination of samples.

### Sample Processing and Sequencing

The rumen fluid samples collected were instantaneously frozen on-site with dry-ice and dispatched the very same day to a storage facility, where they were securely stored at a temperature of −80° C.

When ready for shipment, the frozen rumen fluid samples were brought to room temperature by defrosting them in ZYMO DNA/RNA Shield buffer (ZR-R1100-250). This process aims to safeguard the samples from degradation during transport. They were then transported at room temperature to a specialized DNA service facility.

The DNA was extracted from the rumen fluid samples using the ZymoBIOMICS 96 MagBead DNA Kit (Zymo Research, Cat. no. D4308), in strict adherence to the provided manufacturer's protocol.

Subsequently, library preparation for sequencing was executed strictly adhering to the guidelines provided by the Illumina DNA Prep (Illumina, Cat. no. 20060059). The assembled libraries were then subjected to paired-end sequencing with 2×150 base reads employing an Illumina NovaSeq 6000 instrument. For this purpose, the NovaSeq 6000 S4 Reagent Kit v1.5 (300 cycles) (Illumina, Cat. no. 20028312) was used.

All processes, including DNA extraction, library preparation, and sequencing, were conducted at ZYMO Research based in Freiburg, Germany.

Upon receiving the results of the sequencing process, the integrity of the raw FASTQ sequences was assessed by employing the FASTQC software for quality control, and subsequently, the data was fine-tuned by BBDUK with a set of customized parameters for optimal refinement.

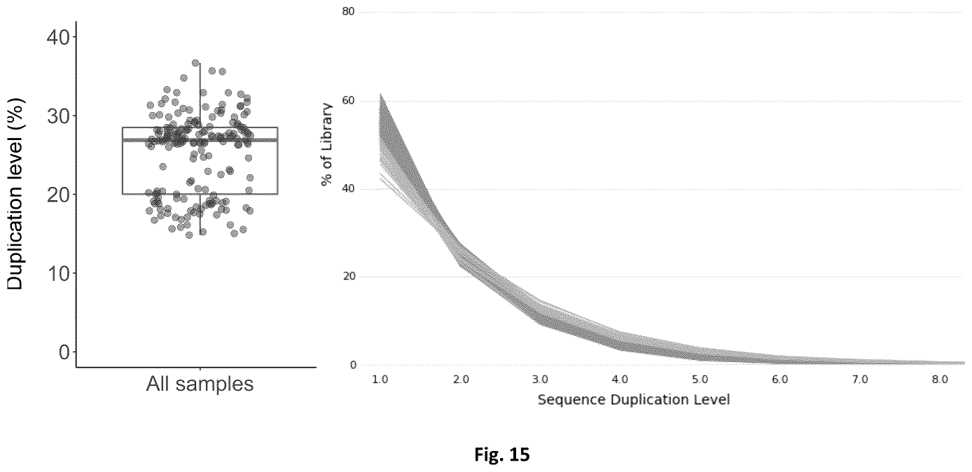

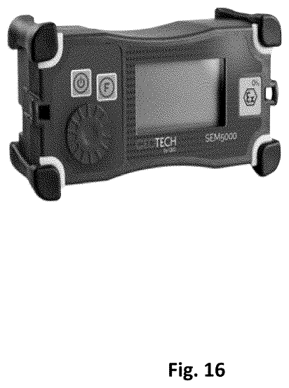

Specifically, Illumina adapters were meticulously eliminated from the 3′ end of the sequence, and all reads that contained fewer than 100 bp were systematically discarded. This data filtering and refinement process resulted in an average yield of approximately 15.7 million reads per sample (see more details in FIG. 16). The average Phred Score for all samples was higher than 35, the adapter content was less than 0.5% with duplication rate of 25% (see FIG. 15).

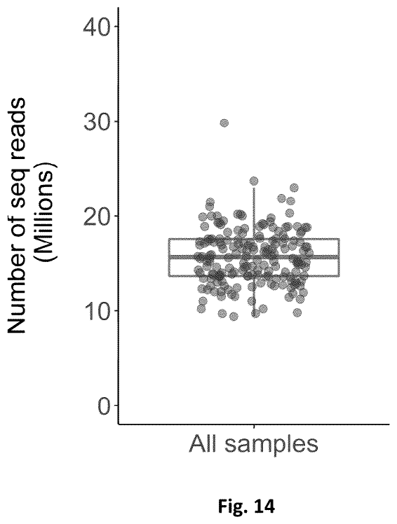

Reference is made to FIG. 14, which is a graph of the sampling depth of sequenced samples across various sequencing processes, according to some embodiments of the invention.

Reference is made to FIG. 15, which is an analysis of sequence duplication levels, illustrating the extent of duplication across various sequencing processes, according to some embodiments of the invention. FIG. 15 shows a majority of samples exhibiting a low count of duplicates, signifying the high quality of the sequencing process.

### Animals

The example hosts used in these experiments were cows, all of the Israeli Holstein breed. There are currently about 102,192 dairy cows in Israel, practically all of which are Israeli Holstein breed. About 70% of all cows are concentrated in Kibbuts herds (large units in cooperatively owned and managed farms), while the remainder belong to Moshav herds (family farms). According to the Israeli Herd Book annual report of 2022 the Israeli cow produced an average of 12,442 kg of milk (production/cow/305 days), of which 3.32% is protein and 3.89% is fat.

### Methane Detection and Quantification

A multitude of methane sensing techniques exists in the market today, reflecting the diverse array of applications they serve from safety monitoring in mining and natural gas industries, air quality surveillance in urban areas, to greenhouse gas emission tracking for climate research. Originally, these devices were not specifically conceived for agricultural settings, let alone for monitoring ruminant animals like cows. However, recognizing the crucial role of livestock in methane emissions, these sensing technologies may be modified and/or used for this purpose. After thorough scrutiny, which considered aspects such as accuracy, durability, suitability for large scale deployment, and adaptability to the unique conditions of a farm environment, sensors were selected for these experiments that were most apt for measuring bovine methane emissions.

### Methane Detection and Measurement

According to some embodiments of the invention, there is provided a sensor designed for measuring methane emissions from cows or other livestock. Location-based tracking of cows is a problem because cows generally live in confined areas with a roof that blocks GPS location tracking. Even for cows roaming in pasture, just taking measures of methane in various locations would not be sufficient, because some embodiments of the invention need to identify to which cow the methane emission measurement relates. In various embodiments, methane emission measurements may be linked with only host ID (but not location, e.g., when measurements are host-specific), only location (but not host ID, e.g., when measurements are farm or region specific), or a combination of host ID and location (e.g., when measurements are herd-specific). Each of these three modes may be used and may be toggled between on the sensor device. Host ID (or device ID of the device worn by, or implanted in, the host) may be stored in the sensor's permanent memory. The sensor may have a Bluetooth based connectivity module that transmits the host ID with each methane emission measurement. The sensor may transmit the data directly to a local device using any short-range wireless technology, such as, Bluetooth, or to a remote device or cloud using any long-range wired or wireless technology, such as, WiFi or cellular networks. The sensor may transmit a methane emission measurement, e.g., periodically, such as, every two seconds, or when there is a significant (e.g., ≥10%) change in levels. Each transmission may thus include host ID and/or host location (depending on the operating mode) and its most current methane emission measurement. In some embodiments, the methane emission measurements for each host (or group of multiple hosts) may be compiled (e.g., from a start time to an end time) and an aggregate value thereof (e.g., the median value) may be calculated, for example, representing the cow's emissions for a predefined period of time (e.g., a day). In some embodiments, prior to aggregating, values below a predefined negligible amount (e.g., 5 parts per million, indicating a non-cow data) may be filtered and deleted from the host's data.

Sensors used according to some embodiments of the invention may include one or more of the following technologies or devices which may be used independently (e.g., different sensors toggled between in different modes of operation) or in combination (e.g., multiple sensor readings collected in parallel as multi-level readings or combined as a single aggregated reading).

Infrared Sensors: These sensors may measure methane concentration by detecting the specific wavelengths absorbed by methane. They tend to be reliable and require low maintenance. Some commercial examples include the ExplorlR-M 5% CO2 Sensor and the SGX Sensortech's IR Methane Sensor. While these sensors are typically quite accurate, their placement and exposure to environmental conditions could impact the readings in a free-range cattle environment.

Semiconductor Sensors: These sensors measure methane by detecting the change in resistance of a semiconductor material exposed to different methane concentrations. These are usually less expensive than infrared sensors, but they tend to have a shorter lifespan and require more maintenance. Figaro's TGS2611 is an example of a semiconductor methane sensor. Their low-cost could be advantageous for wide-scale deployment across large cattle farms.

Catalytic Sensors: These sensors measure methane concentration by detecting the heat produced when methane reacts with a catalyst. However, these sensors might not be ideal for methane measurement from cows due to their susceptibility to poisoning and their requirement for oxygen to function. An example of such a commercial sensor is the Honeywell XCD Methane Gas Detector. This fixed gas detector is designed to provide comprehensive monitoring of combustible gas levels in various environments and is known for its reliability and accuracy.

Electrochemical Sensors: These sensors measure methane by detecting the current generated when methane is oxidized. While they are sensitive and compact, they tend to have a shorter lifespan than other sensor types. The ALTAIR Pro Single-Gas Detector is an example of an electrochemical sensor, designed for worker safety in mind, with a primarily goal to monitor potentially harmful gases in confined spaces.

Photoacoustic Spectroscopy Sensors: Instruments like the INNOVA 1412i Photoacoustic Gas Monitor use the principle of photoacoustic spectroscopy to measure methane emissions. These are highly accurate but can be more expensive and might be more suited to laboratory settings or small scale, intensive research studies.

Laser-based Sensors: Sensors like the LICOR's LI-7700 Open Path CH4 Analyzer use laser technology to measure methane concentrations in the open air. These are highly accurate and can cover a large area, making them suitable for large farms, but they are often also significantly more expensive.

Sulfur Hexafluoride (SF₆) Tracer: This technique is commonly used for measuring methane emissions in ruminants. It involves the animal inhaling a small quantity of SF₆, and the concentration of SF₆ and methane in the exhaled air is measured, allowing for the implicit calculation of the methane production rate of the animal. This technique is widely used in research settings due to its accuracy, but it requires specific equipment and technical expertise, making it less suitable for widespread commercial use. An example is the SF₆ Sulfur Hexafluoride Gas Analyzer by Nova Analytical Systems, specifically designed for such applications.

### Assessment of Methane Emissions

Embodiments of the invention provide techniques that not only accurately represent in-field measurement collection techniques but also minimize the disturbance to the animals. Embodiments of the invention may use methane sensors adapted for bovine applications using one or more of the following data collection methodologies:

Respiration Chambers: The breathing or calorimetric chamber has been the traditional benchmark for measuring CH₄ emissions from ruminants in various settings. This method's aim is to quantify the energy generated through an animal's regular metabolic processes. Such chambers play a role in exploring strategies to curtail CH₄ emissions. They function by monitoring the concentrations of gasses in the animal's exhaled air within a regulated environment. However, the use of the calorimetric chamber is generally confined to the analysis of a single animal due to construction costs and the need for specialized operational skills.

Head chamber: This method typically employs an airtight box, encircling the ruminant's head, with a curtain or sleeve around the neck to restrict air exchange between the internal and external atmospheres of the chamber. The box should be adequately sized to allow unhindered head movements and access to feed and water. Compared to the calorimetric chamber, the prime benefit of this approach lies in its cost-effectiveness (in comparison to the respiration chamber). Similar to the calorimetric chamber, measurements must be performed individually on trained animals.

Face chamber: The face mask, akin to the calorimetric and head chambers, presents another approach for measuring CH₄ from ruminants. This method typically involves fitting a mask onto the animal's head to gather air exhaled through the airways. The animal generally requires a brief acclimation period to the equipment, typically spanning seven days, with e.g., six minute sessions each day. During this time, the animal is typically not allowed to eat or drink, and the analyses are conducted similarly to those in an open calorimetric chamber.

Polyethylene tunnel: This method utilizes a structure reminiscent of an agricultural greenhouse, often erected on a pasture with dual layers of inflatable polyethylene walls and a large entrance. It serves as a simpler alternative to the calorimetric chamber in terms of operation and data collection. Inside this tunnel, air is consistently drawn in, allowing for continuous collection of air samples from an exhaust port for gas analysis or gas chromatography. This method is typically employed to assess CH₄ emissions in areas of fresh forage, allowing animals to behave naturally and controlling selected forage within the confined tunnel space. This technique's benefits include the animals' unrestricted movement within the tunnel and the relatively low acquisition and installation costs. However, it may be impractical to control the tunnel's temperature during periods of high ambient temperature. Most studies using this method have focused on sheep due to pasture space constraints. Additionally, this technique is unsuitable for experiments evaluating various treatments.

Sulfur Hexafluoride (SF₆) Tracer: The sulfur hexafluoride (SF₆) method may involve a small permeation capsule, essentially a metal tube with a porous plate at one end, filled with SF₆. Initially, the capsule is placed in a thermostatic water bath (e.g., for a month) before it is inserted into the animal's rumen. The animal is fitted with a halter that has a capillary tube connected to a PVC yoke. Over a specified duration, this apparatus collects exhaled gases. After a vacuum is applied, the sample is sent to the lab for gas chromatography analysis. A valve in the PVC yoke ensures the collection of exhaled air at a steady rate. This collection system is calibrated to stop once the sample fills approximately half of the system's storage capacity, typically within 24 hours. This method allows the animals to move freely and engage in normal grazing activities, so they are not confined to cages or barometric chambers. Nevertheless, the animals need training to acclimate to the equipment, and the PVC tubing typically requires daily replacement.

Automatic feeder technique (GreenFeed): The GreenFeed technique operates by recognizing an electronic tag on the animal as it begins to feed. The system may then measure the gases emitted every period (e.g., every second) during the feeding process, allowing for the monitoring of individual emission rates over time. Given that approximately 90% of gases produced by ruminants are released through eructation via the mouth and nostrils, this system generates a highly reliable dataset for research on GHG reduction strategies. Upon insertion of its head into the feeder, the animal may be identified via an electronic tag using radio frequency technology (RFID). A fan may then activate to draw in the air exhaled through the animal's nostrils and mouth. Sensors within the equipment measure gas concentrations, the volume of emitted gas, and/or other environmental parameters. Despite its advantages, GreenFeed presents considerable challenges that may limit its use. Its high cost can be prohibitive, especially in larger studies, making implementation unfeasible in many research centers. Additionally, the time required to acclimate animals, particularly Zebu and other native commercial breeds, to the equipment should be considered when planning studies utilizing this technique.

### Tested Sensor

Though several dedicated sensing mechanisms have been developed specifically for monitoring methane emissions from cows, such as respiration chambers, polyethylene tunnel, head chamber, face mask or an automatic feeder, using these tools may result in an alteration of the cows' natural behavior, making the captured data less representative of the animals' day-to-day emission patterns. The disruption of normal behavior is due to the intrusive nature of these devices, which typically require direct contact with the animals or confinement within a restricted space. These methods may also lack scalability. In larger farms with hundreds or thousands of cows, the application of these techniques becomes a logistical challenge, limiting their utility in extensive real-world scenarios. The expenses associated with these techniques further dampen their practicality—the high costs involved in the construction, maintenance, and operation of these devices often make them economically unfeasible for most farms. Furthermore, their use typically demands trained cows which are accustomed to the devices, imposing an additional layer of complexity to the measurement process.

Given these limitations, experiments conducted according to embodiments of the invention used an industrial methane sensor. Industrial sensors are known for their high degree of accuracy and sensitivity, essential features for reliable data collection. More importantly, their non-intrusive nature allows the cows to behave naturally, ensuring that the data gathered is reflective of standard methane emissions under typical conditions. This non-intrusiveness also means the cows require no special training or conditioning to tolerate the device. Being designed for industrial applications, these sensors are robust, cost-effective, and scalable, enabling their usage across larger herds without a significant uptick in operational complexity. Using an adapted industrial sensor aims to bypass many of the hurdles associated with dedicated cow methane sensors and collect reliable and representative data on methane emissions from ruminants.

SEM5000 by Geotech: The ATEX Gas Analyser Geotech SEM5000 (shown in FIG. 16) is a robust, hand-held device specifically designed to measure methane concentrations. Built for use in challenging environments, this device has some key capabilities and advantages that make it well-suited for methane measurement in ruminants like cows. The SEM5000 utilizes laser-based technology for detecting and quantifying methane levels with a range of 0 ppm to 100% volume (laser based sensors are shown to be ideal for methane measurements in ruminants). It boasts a rapid response time, delivering results in seconds. The device also has an in-built GPS, which allows for geo-tagging of measurements and the creation of gas concentration maps. The key advantages of this sensor in methane measurement for cows are as follows:
Non-invasive Method: Unlike some techniques that require animal confinement or behavior modification, the SEM5000 allows for measurements to be taken in a non-invasive manner, reducing stress on the animals and ensuring data gathered represents their natural behavior.
Accuracy and Precision: The laser technology used in the SEM5000 delivers high-accuracy and precision readings, reducing the potential for errors and increasing the reliability of the data.
Portability and Robustness: Given its hand-held design and robust construction, the SEM5000 can be used in a variety of field conditions, making it a practical tool for monitoring methane emissions in grazing environments.
Scalability: The SEM5000 allows for high-throughput data collection and can be used to measure methane emissions from a large number of animals over a short period, making it a more scalable solution than other techniques.
Cost: The SEM5000 Methane Detector is a cost-effective choice, e.g., priced at nearly one-tenth the cost of the Greenfeed system, offering efficient and affordable monitoring of methane emissions.

Reference is made to FIG. 16, which is an image of the Geotech SEM5000 Methane Detector, used according to an embodiment of the invention. The Geotech SEM5000 Methane Detector is a hand-held device utilizing laser-based technology for highly accurate and rapid methane concentration measurements. Its compact design enhances portability and usability under various field conditions, and its fast response time coupled with high data throughput make it an ideal tool for extensive in-field ruminant measurements. The following table 1 shows technical specifications of the Geotech SEM5000 methane detector:

TABLE 1
Technical specifications of the
Geotech SEM5000 methane detector

| Specification | Value | |
|---|---|---|
| Range | 0 to 10,000 ppm | |
| Resolution | 0.1 | ppm |
| Accuracy | 0.7 | ppm |
| Technology | laser based | |
| Response time | <2.5 | sec. |
| Rate | 2 sec. per reading | |
| Battery life | 10 | hours |
| Flow | 1 litter per Min | |
| Weight | 1.3 | kg |

### Comparative Performance

### Evaluation of the SEM5000 Sensor

In order to ascertain the reliability and robustness of the SEM5000 sensor for ruminant methane measurement, a comprehensive study was conducted to show that, although less costly and more scalable than other commonly used technologies, the SEM5000 can deliver equivalent levels of accuracy. For comparative benchmarking, the Li-Cor LI-7810 laser-based system and the GreenFeed system were selected, two established technologies in the field. Li-Cor LI-7810, while robust and accurate, is considerably more expensive, and GreenFeed, though regarded as the gold standard, presents limitations in scalability, usability and cost.

Comparative analysis methodology: A comparison methodology was conducted that involved two distinct sets of tests. In the first set, the same cows were concurrently measured using the SEM5000 and GreenFeed devices. This allowed for a direct comparison between these two methods on the same animal subjects. The mean and median methane emissions were compared from each cow as measured by both sensors. Given the heightened concern over high methane emissions in the context of mitigation, the average of the top 25% of readings were analyzed. To further validate the consistency between the two sensors, the Mann-Whitney U Test was employed, a non-parametric method designed to determine if two sets of readings originate from the same source.

In the second set of tests, the SEM5000 and Li-Cor 7810 devices were used in parallel over a period of four weeks, measuring 48 different cows. This extended period of observation, as well as the large number of specimens being measured, provided a comprehensive set of data to compare the performance of the SEM5000 sensor to the well-established Li-Cor 7810 laser-based system. For each cow, the regression between measurements from the two sensors was determined. Subsequently, the Bland-Altman method was used, a technique tailored for evaluating the concordance of two sensors measuring identical data. As a final step, the Root Mean Square Error (RMSE) was computed to quantify the differences between the two sensors.

The order of measurements was randomized in both sets of tests to minimize potential bias.

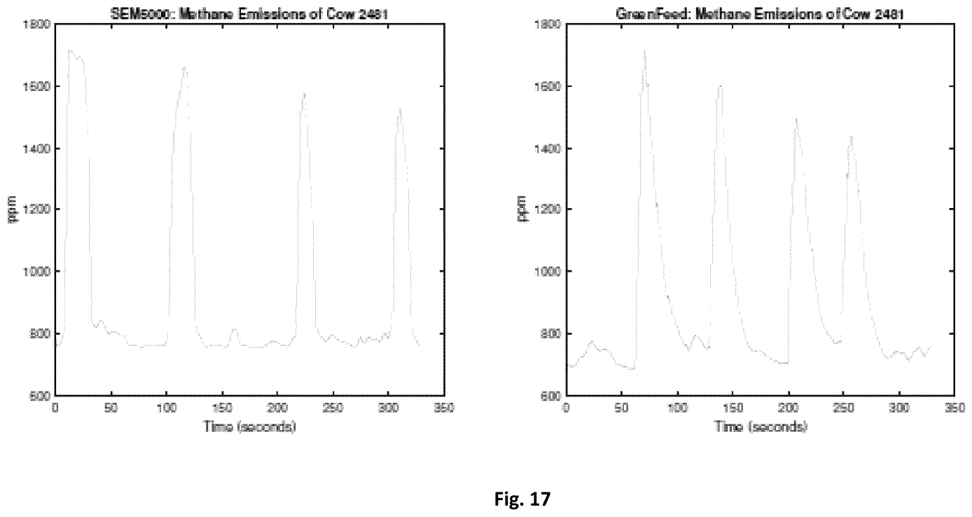

Results: The resulting findings provide compelling evidence supporting the suitability of the SEM5000 sensor for ruminant methane measurement. The comparative data suggest that the SEM5000 sensor achieves high levels of accuracy, comparable to that of the pricier Li-Cor and GreenFeed systems. A detailed analysis of these results is presented in the FIGS. 17-19, demonstrating the commendable performance of the SEM5000 sensor.

Table 2 presents a comparison between measurements taken using the SEM5000 and the Greenfeed sensors for four cows (values are shown as Greenfeed/SEM5000 for each cow).

TABLE 2

| | Cow | Cow | Cow | Cow | Avg. |
|---|---|---|---|---|---|
| Metric | 2071 | 2299 | 2481 | 2849 | Change |
| Mean CH₄ | 159/167 | 123/129 | 909/934 | 127/133 | 4.8% |
| Median CH₄ | 84/87 | 70/73 | 773/782 | 66/68 | 3.6% |
| Mean of top 25% CH₄ | 410/418 | 295/326 | 1318/1397 | 335/328 | 5.6% |
| readings | | | | | |
| STD of CH₄ readings | 196/235 | 123/168 | 261/326 | 161/221 | 29.6% |
| Mann-Whitney U Test p- | 0.065245 | 0.022432 | 0.000019 | 0.009071 | N/A |
| value | | | | | |

The close alignment in Table 2 of these metrics between the two sensors underscores their comparable performance. The Mann-Whitney U Test, a non-parametric statistical test, was employed to determine whether two independent samples were drawn from a population with the same distribution. In this context, the test's results suggest a high probability that the measurements from both sensors are from the same distribution. This conclusion further suggests that data captured using the SEM5000 can, with a high degree of confidence, be used as a proxy for results from the Greenfeed system, paving the way for broader and more flexible deployment of these sensors in methane measurement campaigns.

The results demonstrate that while the SEM5000 sensor exhibits a marginally higher data variance, the key properties, such as average and median emissions levels align closely. The observed differences not only meet the stringent criteria set out by Verra's VM41 protocol and the CDM Meth Panel Guidance on Addressing Uncertainty in the Estimation of Emissions Reductions for CDM Project Activities but also fall below the 15% threshold for sensors' compliance defined in the IPCC 2006 Guidelines, Volume 2, Chapter 2, Tables 2.2 to 2.6. This assertion of compliance is further validated by the Mann-Whitney U Test results, which suggest that (for each cow) both data streams likely derive from the same source. This analysis establishes the credibility of the SEM5000 sensor for measuring enteric methane emissions, affirming its readings to be as dependable as those from the Greenfeed system. The results from the SEM500 and the GreenFeed system, when measuring the same cow, are depicted in FIG. 17. Both measurements showcase comparable emissions levels and temporal dynamics.

The microbiome acquisition and ruminal fluid sampling techniques, sample processing and sequencing techniques, selection of animals, methane detection and quantification techniques, methane detection and measurement techniques and sensors, and assessment of methane emissions techniques disclosed herein are all non-limiting embodiments used only for examples. Other techniques and variations of those disclosed may also be used according to embodiments of the invention.

Reference is made to FIG. 17, which includes two graphs of methane levels (in parts per million) for the same cow, as measured at different times by the SEM5000 sensor and the GreenFeed system, used according to an embodiment of the invention. The readings in the two graphs exhibit similar dynamics and values.

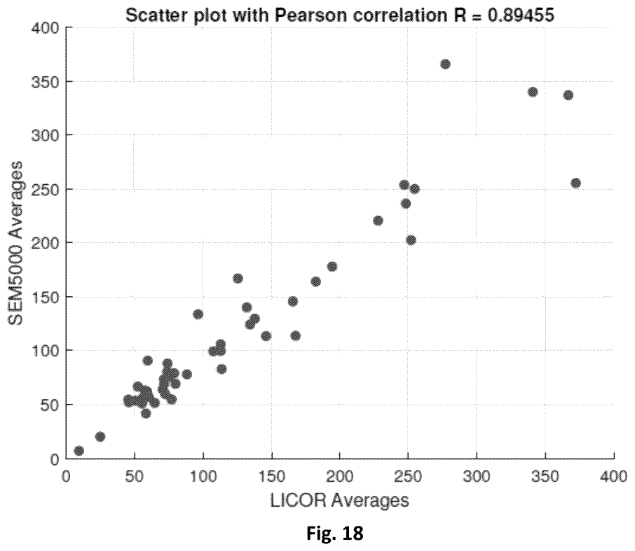

Reference is made to FIG. 18, which is a graph comparing readings from the SEM5000 sensor and the LICOR 7810 laser-based sensor, used according to an embodiment of the invention. Each point represents the averaged methane emissions from a unique cow, as measured by both sensors. The Pearson regression line is also depicted, with an accompanying R² value, indicating the strength and direction of the linear relationship between the two sets of measurements. This high correlation implies that the two sensors produce consistent and comparable results, further validating the reliability and accuracy of the SEM5000 in measuring enteric methane emissions. The average discrepancy (noise) between measurements from the two sensors was found to be 14%, with a median discrepancy of 10%. Additionally, the Root Mean Square Error (RMSE) between their measurements stood at 28.

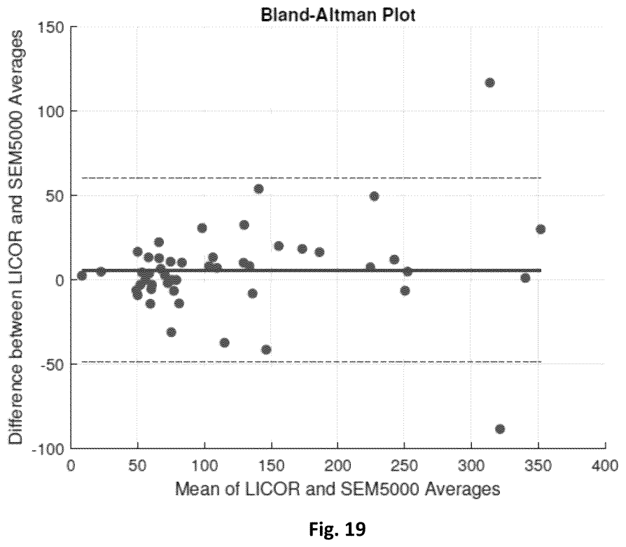

Reference is made to FIG. 19, which is a graph showing a Bland-Altman analysis comparing the SEM5000 and the LICOR 7810 laser-based sensor, used according to an embodiment of the invention. The Bland-Altman test is used to assess the agreement between two different instruments measuring the same parameter. In the plot, the difference between the two sensors' readings is plotted against their average. The central line represents the mean difference, while the outer lines depict the limits of agreement, which are calculated as the mean difference ±1.96 times the standard deviation of the differences. Apart from two outliers, all data points lie within the confidence limits indicating that the measurements from the two sensors are largely in agreement and can be used interchangeably for most practical purposes.

FIGS. 18 and 19 further bolster the credibility of the SEM5000 sensor for enteric methane measurements. In these figures, methane emissions from 48 distinct cows, measured over a span of 4 weeks by both sensors, are showcased. A discernible strong correlation between the measurements from the two sensors is evident. Coupled with the robust correlation, the Bland-Altman analysis further corroborates these observations. The BlandAltman method is primarily utilized to assess the agreement between two different measurement techniques, determining the consistency and discrepancy in results. Its affirmation in this context emphasizes the reliability and similarity of readings between the two sensors.

### Periodic Methane Measurement: Duration and Consistency

Accuracy of experiments conducted according to embodiments of the invention was bolstered by the repeated measurements of each cow throughout an extended 12-week period post-treatment. This strategy is rooted in the need to validate the persistent efficacy—or potential lack thereof—of the additives. Over time, factors such as changes in feed quality, external environmental conditions, or the cow's inherent physiology may impact methane emission levels. By measuring emissions repeatedly over several weeks, these experiments ensure that the observed effects (or non-effects) of the additive remain consistent.

The importance of extended measurement periods is further underscored by standards set by external bodies. Specifically, the VM41 protocol for enteric methane measurement and reduction, established by the Verra agency, mandates that projects measure emissions for at least 8 weeks to be compliant with its guidelines. This timeframe is recommended to ensure a thorough assessment of the additive's performance. In acknowledgment of the significance of these guidelines, experiments conducted according to embodiments of the invention aim to enhance the statistical robustness of the results, by measuring data over an extended duration of 12-weeks.

While experiments may benefit from measuring every cow in both the treatment and control groups during each farm visit, such rigor poses the inherent challenge of occasional evasiveness from some cows, preventing them from being tied and measured. This natural occurrence is an inevitable challenge of working with live animals in a field setting that encourages the implementation of flexible animal and data handling strategies.

At each time point in the experiment, the efficacy of the feed additive was calculated by comparing the current measurements of the cows that were available and measured on that day to their emission levels recorded before the commencement of the trial. This implies that the exact composition of cows measured may differ between consecutive farm visits. However, this does not compromise the accuracy of the efficacy calculation at each point because the cows in both the treatment and control groups are compared to their individual baseline emissions level, which was established prior to the initiation of the trial. This procedure implies the efficacy determination is based on individualized comparisons, thereby maintaining the overall reliability of the results.

Procedures were implemented to manage the potential variability in measurements due to cow evasiveness. The percentage of cows that managed to evade measurement was consistently kept below 10%, reducing the overall impact of this phenomenon on our data set. Consequently, the influence of this variability on our overall findings is likely minimal. This aspect of the study underscores the complexities of field research in livestock environments and the need for adaptable research methodologies.

### Methane Reading Methodology

When measuring methane emissions from cows, the variability in the data due to diverse observation durations and transient spikes presents challenges. The methodology may address these discrepancies to yield reliable and consistent metrics.

Ambient Noise Filtering: Methane measurements can capture ambient readings, especially before and after the actual approach to the cow. To filter out these irrelevant readings and hone in on the cow's emissions, only values above 5 parts per million (ppm) were considered. This threshold ensures focus on the cow's emissions, excluding most ambient interference.

Data Consolidation and Noise Reduction: To condense the varied readings from each visit into a single representative number and simultaneously mitigate the noise (like sudden spikes due to burping), median metrics were used. Asa measure of central tendency, the median may be robust against outliers, offering a more stable representation of the cow's typical methane emission.

Formally, given a set of methane readings for a specific cow on a specific day, e.g.: {Rt₁, Rt₂, . . . , Rt_{N}}, the consolidated value for that day and cow may be computed as, e.g.:

$$\overline{R} = median ( \{ {R}_{t 1} \vert {R}_{t 2} > 5 ppm \} )$$

By implementing this methodology, a single, consistent methane reading is obtained per cow for each visit, establishing a dependable foundation for subsequent comparative analysis across various visits and cows.

### Feed Additives

The landscape of feed additives, designed to mitigate methane emissions, is rich and varied, with each product leveraging a unique biological strategy. These formulations are designed to interact with the bovine digestive process in various ways to reduce the production of methane, a major byproduct. The efficacy of these additives is largely determined by the specific biological pathway they target, underlining the need for personalized application based on each farm's specific conditions and requirements. From methane inhibitors and direct-fed microbials to natural plant extracts and chemical compounds, the range of solutions showcases the vast scope of scientific novelty directed towards curbing this environmental concern. Experiments test the ability to predict the efficacy of the following widely used and commercially available additives.

Agolin: Ruminant (Agolin) is a commercially available blend of essential oils (coriander seed oil, eugenol, geranyl acetate, and geraniol) which has been demonstrated to reduce greenhouse gas emissions in dairy cows and improve energy corrected milk and feed efficiency at a daily dose of 0.8 to 1 gram per animal. Agolin increases milk production in cows producing moderate milk yield (30 kg/d), however, this response depends on duration of feeding (5 to 8 week minimum). Some observed a consistent and convincing 2-3% increase in yields of milk or ECM. Agolin is shown to inhibit ruminal methane production or intensity by 8% on average while no apparent change in dry matter intake (DMI) nor on milk composition was described. An exact mode of action is yet to be elucidated.

Relyon: Manufactured by Phibro Animal Health, this tannins flavonoid and essential oils-based additive was shown to mitigate ruminal methene emission by 13% on average, while no change in milk yield or its composition was observed. While more rigorous scientific studies are desirable to substantiate Relyon's promising role in also enhancing feed conversion and stimulating appetite in ruminants, the preliminary results presented to date are encouraging.

Kexxtone (Elanco): Kexxtone is a Monensin containing intraruminal bolus for administration 3-4 weeks pre-calving to help the peri-parturient dairy cow/heifer maintain an appropriate energy balance and thereby preventing many periparturient metabolic based diseases. The Kexxtone bolus releases Monensin for a period of 95 days in the rumen. Ionophores such as Monensin improve methane mitigation by enhancing digestive efficiency to favor propionate production over acetate, which reduces H₂ for methanogens. This methanogenesis inhibition becomes more pronounced in diets with higher fat content. Meta-analyses of Monensin conclude an effect on methanogenesis inhibition of up to 10% reduction on average in dairy.

Allimax: Allimax bolus (Garlic, Allicin) has been developed for the purpose of alternative antimicrobial activity in dairy. The natural extract Allicin, which is the main active ingredient of the sulfur-containing organic compounds in garlic, has anti-inflammatory, anticancer, antioxidant, and antibacterial properties. However, the specific mechanism underlying its effect on mastitis in dairy cows needs to be further studied. The supplementation of Allicin has been observed to elevate the levels of propionate and butyrate during partial incubation periods, suggesting its potential role in curtailing methane emissions. Even though compelling in vitro evidence demonstrates the ability of Allicin to mitigate methane emissions by up to 38%, in vivo studies confirming these findings remain scarce to date.

### From Microbial Data to Additive Efficacy Prediction

Some embodiments of the invention may operate for example according to the following descriptions and definitions related to data handling, validation, training and prediction.

Input: Throughout experiments, researchers collaborated with 20 trial farms, from each of which 15 microbiome samples were collected and sent for deep shotgun metagenomic sequencing. Moreover, each tested additive was administered to 20 cows, with an additional group of 20 cows selected as a control group, which received no treatment. Methane emissions were measured from each cow periodically, for calculating the efficacy of each additive on each farm.

Stage 1 Unsupervised Detection of Microbial DNA Patterns: Experiments used the raw data obtained from the sequencing strings of 100-150 nucleotides, without undertaking any identification of microbes or strains. Network-oriented DNA analytics according to embodiments of the invention were used to analyze the data from all collected microbiome samples. This resulted in numerous DNA patterns, each analytically determined to be unlikely to appear spontaneously in random genetic microbial samplings, and therefore likely associated with a phenotypic property. Stage 1 may function as a potent dimensionality reduction process, capable of operating on billions of raw 100-150 base-long strings. It may extract a computationally manageable number of groups of “statistically meaningful” substrings, avoiding bias towards predefined feature spaces, data pre-processing, or the semantics of the problem at hand. Stage 1 may be updated as new data becomes available, leading to the identification of new DNA patterns that contribute to the system's predictive capabilities for the same or new properties.

Stage 2 Filtering the Microbial DNA Patterns using Semantic Labels: For each DNA pattern (e.g., a collection of 100-150 long DNA bases), embodiments of the invention may filter only those whose frequency (e.g., the number of times they occur in a sample) correlates strongly with the biological condition to be predicted (e.g., an additive's efficacy, defined by its methane reduction capacity, normalized for the control group on the same farm). Stage 2 may be executed once for each group of labels (e.g., once per additive).

Output: Embodiments of the invention may output a set of groups of DNA sequences, each statistically shown to be associated with a target biological condition with some weight. Additionally, the output may include a formula to combine these weights with the relative frequency of these sequences in a given microbiome sample, generating a final aggregated score, such as, between 0 (no efficacy) and 1 (maximal efficacy for this additive). Once this biomarker output is available, it can be applied to future microbiome samples directly, eliminating the need to re-run the previous steps.

Microbiome Biomarkers Used in this Study

Embodiments of the invention provide a unique artificial intelligence-based data analytics technique that accepts sequenced microbial data alongside associated labels for a specific biological condition and generates a predictive model for that biological condition applicable to future microbiome samples. This innovative method is generic, in that it can generate a ‘biomarker’ from any attribute presented in label form (for example, a set of microbiome samples associated with high efficacy of a particular feed additive). This biomarker may comprise a limited number of short DNA sequences, the presence levels of which in microbiome samples can predict the given biological condition.

Table 3 lists biomarkers of groups of microbiome DNA segments of one or more livestock hosts identified according to embodiments of the invention to be correlated with a biological condition of one or more livestock hosts. In some embodiments, these DNA segments are associated with the respective weights and regression formula. The example shown in Table 3 lists 8 groups of k-mer DNA segments, where each group of k-mers was tested and detected to exhibit a significant correlation with high efficacy (“Top” k-mers) and low efficacy (“Bottom” k-mers) for reduced methane emission in cows fed the additive listed (Relyon, Agolin, Allimax, and Kexxtone). For each additive, the group of “Top” k-mers are indicative of high expected efficacy (strongest association with high efficacy) and the group of “Bottom” k-mers are indicative of low expected efficacy (strongest association with lowest efficacy). These k-mer groups can be used to predict which hosts will reduce their methane emission upon consuming the correlated feed additive. In one embodiment, the correlation between k-mer groups and a biological condition (e.g., the efficacy of an additive for reducing methane emissions) across a group of hosts (e.g., a farm-level score for a group of sampled cows in a farm F) may be computed, for example, as follows:
1. Let F be a farm, and let A be an additive administered to cows to predict its efficacy for farm F.
2. For each C, a cow of farm F, calculate the top-1000 most frequently occurring 30-mers in its biological sample.
3. The score of cow C may be calculated e.g., as (C_top-C_bottom), where:
   a. C_top=(number of k-mers from the “Top” list in Table 3 for additive A that are part of the top-1000 most popular k-mers for cow C) divided by (length of the “Top” list for additive A). This yields numbers between 0 (no presence in top-1000) and 1 (all k-mers are present in top-1000).
   b. C_bottom=(number of k-mers from the “Bottom” list for additive A that are part of the top-1000 most frequently occurring k-mers for cow C) divided by (length of “bottom” list for additive A). This yields numbers between 0 and 1 Note that every cow may have a score between 1 and −1.
4. For the farm F, average the scores of all of its sampled cows. Yielding a score for the farm level, that is between −1 and 1. To this score, add “1” and then divide by “2” to normalize between 0 (expected low efficacy) and 1 (expected high efficacy). V_{\hat{d}} lues around 0.5 may indicate there is not enough information to predict expected efficacy.
   In other embodiments, the correlation between k-mer groups and a biological condition may be predicted by Machine Learning models.

TABLE 3

| Additive | Top k-mers | Bottom k-mers |
|---|---|---|
| Relyon | AATCATGCTGCTCAGCTGGCAATAATCAAG | AATACCCAAAACCCAAAACCCAAAACCCAA |
| | (SEQ ID NO: 1) | (SEQ ID NO: 37) |
| | AATCTTCCATTCGAGTTGCGAAGGAAAGCT | AATCCCCAATCCCCAAAACCCAAAACCCCA |
| | (SEQ ID NO: 2) | (SEQ ID NO: 38) |
| | ACACACACACACACACACACACACACACAC | AATTTTAATACTAATAATGTAACTAATATG |
| | (SEQ ID NO: 3) | (SEQ ID NO: 39) |
| | ACCTGCCCGCTCATCTGCCTGATACTCGCC | AGAGCAGAGCAGAGCAGAGCAGAGCAGAGC |
| | (SEQ ID NO: 4) | (SEQ ID NO: 40) |
| | ACGTGATCAGTGCATGATCAGTCACGTGAT | ATAATAATAATAATAATAATAATAATAATA |
| | (SEQ ID NO: 5) | (SEQ ID NO: 41) |
| | ACTGACCTCGCCGTCGTACCTCGTGAGAAA | ATATTAGTTACATTATTAGTATTAAAATTA |
| | (SEQ ID NO: 6) | (SEQ ID NO: 42) |
| | AGGTGTCGCGCGGCTCAGCTGGCGAGTATC | ATTATTATTATTATTATTATTATTATTATT |
| | (SEQ ID NO: 7) | (SEQ ID NO: 43) |
| | AGTATCAGGCAGATGAGCGGGCAGGTGTCG | ATTGGGCCCAATCCCCAATCCCCAAACCCC |
| | (SEQ ID NO: 8) | (SEQ ID NO: 44) |
| | AGTGCATGATAGCCACGTGATCAGTGCATG | ATTGGGGATTGGGGATTGGGGAGTGGGGAT |
| | (SEQ ID NO: 9) | (SEQ ID NO: 45) |
| | ATAGCCACGTGATCAGTGCATGATCAGTCA | CAATCCCCAAAACCCAAAACCCCAAACCCC |
| | (SEQ ID NO: 10) | (SEQ ID NO: 46) |
| | ATCACGTGACTGATCATGCACTGATCACGT | CACTGACTGCAGTGATAACACTGACTGCAG |
| | (SEQ ID NO: 11) | (SEQ ID NO: 47) |
| | ATCAGGCAGATGAGCGGGCAGGTGTCGCGC | CCAATCCCCAATCCCCAAACCCCAATCCCC |
| | (SEQ ID NO: 12) | (SEQ ID NO: 48) |
| | ATCAGTGCATGATAGCCACGTGATCAGTGC | CCAATCCCCAATCCCCAAACCCCCAAACCC |
| | (SEQ ID NO: 13) | (SEQ ID NO: 49) |
| | ATCATGCACTGATCACGTGACTGATCATGC | CCAATCCCCAATCCCCAATACCCAAAACCC |
| | (SEQ ID NO: 14) | (SEQ ID NO: 50) |
| | ATCATGCACTGATCACGTGGCTATCATGCA | CCCAATCCCCAATCCCCAATCCCCAATACC |
| | (SEQ ID NO: 15) | (SEQ ID NO: 51) |
| | ATCATGCACTGATCACGTGGCTGATCATAC | CCCCAATCCCCAAAACCCAAAACCCCAAAC |
| | (SEQ ID NO: 16) | (SEQ ID NO: 52) |
| | ATGATAGCCACGTGATCAGTGCATGATCAG | CCCCAATCCCCAATCCCCAAAACCCAAAAC |
| | (SEQ ID NO: 17) | (SEQ ID NO: 53) |
| | ATGATCAGTCACGTGATCAGTGCATGATCA | CTTATACACATCTCGAGCCCACGAGACACT |
| | (SEQ ID NO: 18) | (SEQ ID NO: 54) |
| | ATGCACTGATCACGTGGCTATCATGCACTG | CTTATACACATCTCGAGCCCACGAGACCTA |
| | (SEQ ID NO: 19) | (SEQ ID NO: 55) |
| | ATGCACTGATCACGTGGCTGATCATACACT | CTTATACACATCTCGAGCCCACGAGACGCT |
| | (SEQ ID NO: 20) | (SEQ ID NO: 56) |
| | CAGCTGGCGAGTATCAGGCAGATGAGCGGG | GACTGCAGTGATAACACTGACTGCAGTGAT |
| | (SEQ ID NO: 21) | (SEQ ID NO: 57) |
| | CATGATCAGTCACGTGATCTGTGCATGATC | GATAACACTGACTGCAGTGATAACACTGAC |
| | (SEQ ID NO: 22) | (SEQ ID NO: 58) |
| | CGCGGCTCAGCTGGCGAGTATCAGGCAGAT | GATTGGGGATTGGGGAGTGGGGATTGGGGA |
| | (SEQ ID NO: 23) | (SEQ ID NO: 59) |
| | CGTGATCAGTGCATGATAGCCACGTGATCA | GGGATTGGGGATTGGGGATTGGGGAGTGGG |
| | (SEQ ID NO: 24) | (SEQ ID NO: 60) |
| | CTCATCTGCCTGATACTCGCCAGCTGAGCC | GGGGATTGGGGATTGGGGAGTGGGGATTGG |
| | (SEQ ID NO: 25) | (SEQ ID NO: 61) |
| | CTGATCATGCACTGATCACGTGGCTATCAT | TATTGGGGATTGGGGATTGGGGATTGGGGA |
| | (SEQ ID NO: 26) | (SEQ ID NO: 62) |
| | CTTCCATTCGAGTTGCGAAGGAAAGCTGGG | TGCTTGCTTGCTTGCTTGCTTGCTTGCTTG |
| | (SEQ ID NO: 27) | (SEQ ID NO: 63) |
| | GCATGATCAGCCACGTGATCAGTGCATGAT | TTGGGGAGTGGGGATTGGGGATTGGGGATT |
| | (SEQ ID NO: 28) | (SEQ ID NO: 64) |
| | GCGAGTATCAGGCAGATGAGCGGGCAGGTG | |
| | (SEQ ID NO: 29) | |
| | GCTCAGCTGGCGAGTATCAGGCAGATGAGC | |
| | (SEQ ID NO: 30) | |
| | GGCAGGTGTCGCGCGGCTCAGCTGGCGAGT | |
| | (SEQ ID NO: 31) | |
| | GTGCATGATCAGTCACGTGATCTGTGCATG | |
| | (SEQ ID NO: 32) | |
| | GTGTGTGTGTGTGTGTGTGTGTGTGTGTGT | |
| | (SEQ ID NO: 33) | |
| | TCATGCACTGATCACGTGGCTGATCATGCA | |
| | (SEQ ID NO: 34) | |
| | TGCATGATCAGTCACGTGATCAGTGCATGA | |
| | (SEQ ID NO: 35) | |
| | TGTCGCGCGGCTCAGCTGGCGAGTATCAGG | |
| | (SEQ ID NO: 36) | |
| Agolin | ACGTGATCAGTGCATGATCAGTCACGTGAT | AAAGGTACGAAAATTTTAGCTAATCACAAC |
| | (SEQ ID NO: 65) | (SEQ ID NO: 90) |
| | AGGTGTCGCGCGGCTCAGCTGGCGAGTATC | ACCTTGCAAAGGTACGAAAATTTTAGCTAA |
| | (SEQ ID NO: 66) | (SEQ ID NO: 91) |
| | AGTATCAGGCAGATGAGCGGGCAGGTGTCG | ACGCGTGGACGCGTGGACGCGTGGACGCGT |
| | (SEQ ID NO: 67) | (SEQ ID NO: 92) |
| | AGTGCATGATAGCCACGTGATCAGTGCATG | ATAATAATAATAATAATAATAATAATAATA |
| | (SEQ ID NO: 68) | (SEQ ID NO: 93) |
| | ATAGCCACGTGATCAGTGCATGATCAGTCA | ATGACCTTGCAAAGGTACGAAAATTTTAGC |
| | (SEQ ID NO: 69) | (SEQ ID NO: 94) |
| | ATCAGTGCATGATAGCCACGTGATCAGTGC | CGTGGACGCGTGGACGCGTGGACGCGTGGA |
| | (SEQ ID NO: 70) | (SEQ ID NO: 95) |
| | ATCATGCACTGATCACGTGACTGATCATGC | CTTATACACATCTCGAGCCCACGAGACCTA |
| | (SEQ ID NO: 71) | (SEQ ID NO: 96) |
| | ATCATGCACTGATCACGTGGCTATCATGCA | CTTATACACATCTCGAGCCCACGAGACGCT |
| | (SEQ ID NO: 72) | (SEQ ID NO: 97) |
| | ATGATAGCCACGTGATCAGTGCATGATCAG | GACGCATGACGCATGACGCATGACGCATGA |
| | (SEQ ID NO: 73) | (SEQ ID NO: 98) |
| | ATGATCAGTCACGTGATCAGTGCATGATCA | GCCAAGCTGTTCTTGGCGTAAGATGCAATG |
| | (SEQ ID NO: 74) | (SEQ ID NO: 99) |
| | ATGCACTGATCACGTGGCTATCATGCACTG | GCGTAAGATGCAATGGCTGAGAACTTGACT |
| | (SEQ ID NO: 75) | (SEQ ID NO: 100) |
| | ATTGGGGATTGGGGATTGGGGATTGGGGAT | GCTGAGAACTTGACTTTCAAGAGTTCTTTT |
| | (SEQ ID NO: 76) | (SEQ ID NO: 101) |
| | CAGCTGGCGAGTATCAGGCAGATGAGCGGG | GCTGTTCTTGGCGTAAGATGCAATGGCTGA |
| | (SEQ ID NO: 77) | (SEQ ID NO: 102) |
| | CGCGGCTCAGCTGGCGAGTATCAGGCAGAT | GTTCTTGGCGTAAGATGCAATGGCTGAGAA |
| | (SEQ ID NO: 78) | (SEQ ID NO: 103) |
| | CGTGATCAGTGCATGATAGCCACGTGATCA | GTTGAGAGTTGAGAGTTGAGAGTTGAGAGT |
| | (SEQ ID NO: 79) | (SEQ ID NO: 104) |
| | CTCATCTGCCTGATACTCGCCAGCTGAGCC | GTTGATGACCTTGCAAAGGTACGAAAATTT |
| | (SEQ ID NO: 80) | (SEQ ID NO: 105) |
| | GCATGATCAGCCACGTGATCAGTGCATGAT | TAAGATGCAATGGCTGAGAACTTGACTTTC |
| | (SEQ ID NO: 81) | (SEQ ID NO: 106) |
| | GCGAGTATCAGGCAGATGAGCGGGCAGGTG | TAGGCCAAGCTGTTCTTGGCGTAAGATGCA |
| | (SEQ ID NO: 82) | (SEQ ID NO: 107) |
| | GCTCAGCTGGCGAGTATCAGGCAGATGAGC | TCATGCGTCATGCGTCATGCGTCATGCGTC |
| | (SEQ ID NO: 83) | (SEQ ID NO: 108) |
| | GGCAGGTGTCGCGCGGCTCAGCTGGCGAGT | TCTCTTATACACATCTACGCTGCCGACGAC |
| | (SEQ ID NO: 84) | (SEQ ID NO: 109) |
| | GGGATTGGGGATTGGGGATTGGGGATTGGG | TCTTATACACATCTCCAGCCCACGAGACTT |
| | (SEQ ID NO: 85) | (SEQ ID NO: 110) |
| | GTGCATGATCAGTCACGTGATCAGTGCATG | TCTTATACACATCTCGAGCCCACGAGACTT |
| | (SEQ ID NO: 86) | (SEQ ID NO: 111) |
| | GTGTGTGTGTGTGTGTGTGTGTGTGTGTGT | TCTTATACACATCTTGACGCTGCCGACGAC |
| | (SEQ ID NO: 87) | (SEQ ID NO: 112) |
| | TCATGCACTGATCACGTGGCTGATCATGCA | TGCAAAGGTACGAAAATTTTAGCTAATCAC |
| | (SEQ ID NO: 88) | (SEQ ID NO: 113) |
| | TGTCGCGCGGCTCAGCTGGCGAGTATCAGG | TGCAATGGCTGAGAACTTGACTTTCAAGAG |
| | (SEQ ID NO: 89) | (SEQ ID NO: 114) |
| | | TGTCAAGCGGCAACCGATCGGTTACGCTGA |
| | | (SEQ ID NO: 115) |
| | | TTATCTCATTGCTTTTCACCTCACACATTT |
| | | (SEQ ID NO: 116) |
| | | TTCAAGAGTTCTTTTCTCTTTCTGATTGCC |
| | | (SEQ ID NO: 117) |
| | | TTCACCTCACACATTTCAGTGTCAAGCGGC |
| | | (SEQ ID NO: 118) |
| | | TTCAGTGTCAAGCGGCAACCGATCGGTTAC |
| | | (SEQ ID NO: 119) |
| | | TTGACTTTCAAGAGTTCTTTTCTCTTTCTG |
| | | (SEQ ID NO: 120) |
| | | TTGCTTTTCACCTCACACATTTCAGTGTCA |
| | | (SEQ ID NO: 121) |
| | | TTGGCGTAAGATGCAATGGCTGAGAACTTG |
| | | (SEQ ID NO: 122) |
| Allimax | AAACATGGGCAGGCCTATGAAACCCACCGC | AAAATTAGATAAATTTAAAGAAGTTAAAGA |
| | (SEQ ID NO: 123) | (SEQ ID NO: 142) |
| | AAAGAGAGGTGAGAAACATGGGCAGGCCTA | AACATTATTAGTATTAAAATTAGATAAATT |
| | (SEQ ID NO: 124) | (SEQ ID NO: 143) |
| | AAATTAATGTTTATATATGTTAAATTAATG | AATAATAATAATAATAATAATAATAATAAT |
| | (SEQ ID NO: 125) | (SEQ ID NO: 144) |
| | AACGCTGTACAAGAAGCGCCTGAACACCGA | AATCCCCAATCCCCAAAACCCAAAACCCAA |
| | (SEQ ID NO: 126) | (SEQ ID NO: 145) |
| | ACGCATGACGCATGACGCATGACGCATGAC | AATGGGGATTGGGGATTGGGGATTGGGGAT |
| | (SEQ ID NO: 127) | (SEQ ID NO: 146) |
| | ATATGTTAAATTAATGTTTATATATGTTAA | AATTGGGGATTGGGGATTGGGGATTGGGGA |
| | (SEQ ID NO: 128) | (SEQ ID NO: 147) |
| | ATGACGCATGACGCATGACGCATGACGCAT | AGATAAATTTAAAGAAGTTAAAGAAGAACA |
| | (SEQ ID NO: 129) | (SEQ ID NO: 148) |
| | ATGCGTCATGCGTCATGCGTCATGCGTCAT | AGTATTAAAATTAGATAAATTTAAAGAAGT |
| | (SEQ ID NO: 130) | (SEQ ID NO: 149) |
| | ATGGGCAGGCCTATGAAACCCACCGCAGTC | ATTAAAATTAGATAAATTTAAAGAAGTTAA |
| | (SEQ ID NO: 131) | (SEQ ID NO: 150) |
| | CAGGCCTATGAAACCCACCGCAGTCAAGAA | ATTAGATAAATTTAAAGAAGTTAAAGAAGA |
| | (SEQ ID NO: 132) | (SEQ ID NO: 151) |
| | CCAGACCCTCAGCGACATCGGAACGACCGC | ATTAGTATTAAAATTAGATAAATTTAAAGA |
| | (SEQ ID NO: 133) | (SEQ ID NO: 152) |
| | CGTCATGCGTCATGCGTCATGCGTCATGCG | ATTATTAGTATTAAAATTAGATAAATTTAA |
| | (SEQ ID NO: 134) | (SEQ ID NO: 153) |
| | CTCTGCTCTGCTCTGCTCTGCTCTGCTCTG | ATTATTATTATTATTATTATTATTATTATT |
| | (SEQ ID NO: 135) | (SEQ ID NO: 154) |
| | CTCTTATACACATCTCGAGCCCACGAGACA | ATTGGGCCCAATCCCCAATCCCCAAACCCC |
| | (SEQ ID NO: 136) | (SEQ ID NO: 155) |
| | GAGAAACATGGGCAGGCCTATGAAACCCAC | ATTGGGGATTGGGGATTGGGGATTGGGCCC |
| | (SEQ ID NO: 137) | (SEQ ID NO: 156) |
| | GGTGAGAAACATGGGCAGGCCTATGAAACC | CCAATCCCCAAAACCCAAAACCCCAAACCC |
| | (SEQ ID NO: 138) | (SEQ ID NO: 157) |
| | GTTAAATTAATGTTTATATATGTTAAATTA | CCAATCCCCAATCCCCAATACCCAAAACCC |
| | (SEQ ID NO: 139) | (SEQ ID NO: 158) |
| | TCTTTTTCTTTTTCTTTTTCTTTTTCTTTT | CCAATCCCCAATCCCCAATCCCCAATCCCC |
| | (SEQ ID NO: 140) | (SEQ ID NO: 159) |
| | TTTTCTTTTTCTTTTTCTTTTTCTTTTTCT | CCCAATCCCCAATCCCCAAAACCCAAAACC |
| | (SEQ ID NO: 141) | (SEQ ID NO: 160) |
| | | GATTGGGGATTGGGGATTGGGGATTGGGGG |
| | | (SEQ ID NO: 161) |
| | | GGGATTGGGGAGTGGGGATTGGGGATTGGG |
| | | (SEQ ID NO: 162) |
| | | GGGGATTGGGGATTGGGGATTGGGGATTGG |
| | | (SEQ ID NO: 163) |
| | | TAAATTTAAAGAAGTTAAAGAAGAACAATT |
| | | (SEQ ID NO: 164) |
| | | TCCCCAATCCCCAATCCCCAATCCCCATTA |
| | | (SEQ ID NO: 165) |
| | | TCTTATACACATCTCGAGCCCACGAGACGA |
| | | (SEQ ID NO: 166) |
| | | TGGGGATTGGGGATTGGGGAGTGGGGATTG |
| | | (SEQ ID NO: 167) |
| | | TTGGGGATTGGGGAGTGGGGATTGGGGATT |
| | | (SEQ ID NO: 168) |
| | | TTGGGGATTGGGGATTGGGGATTGGGGCCA |
| | | (SEQ ID NO: 169) |
| Kexxtone | AAACACCATATATATTGAGAAAGAGAGGTG | AAACGCCTCAGGAGGCTTGACTCCCTTGAG |
| | (SEQ ID NO: 170) | (SEQ ID NO: 199) |
| | AAACATGGGCAGGCCTATGAAACCCACCGC | AAGAAGAAGAAGAAGAAGAAGAAGAAGAAG |
| | (SEQ ID NO: 171) | (SEQ ID NO: 200) |
| | AAATTAATGTTTATATATGTTAAATTAATG | AGGTACGACGGCGAGGTCAGTGAGCCTCTC |
| | (SEQ ID NO: 172) | (SEQ ID NO: 201) |
| | AAATTTAAAGAAGTTAAAGAAGAACAATTA | AGTGCATGATAGCCACGTGATCAGTGCATG |
| | (SEQ ID NO: 173) | (SEQ ID NO: 202) |
| | AAATTTATCTAATTTTAATACTAATAATGT | ATCAGTGCATGATAGCCACGTGATCAGTGC |
| | (SEQ ID NO: 174) | (SEQ ID NO: 203) |
| | AACTTCTTTAAATTTATCTAATTTTAATAC | ATCATGCACTGATCACGTGACTGATCATGC |
| | (SEQ ID NO: 175) | (SEQ ID NO: 204) |
| | AAGAAGAAGAAGAAGAAGAAGTTGAACATG | ATCTCGCGACCTCTCTCCAAACGCCTCAGG |
| | (SEQ ID NO: 176) | (SEQ ID NO: 205) |
| | AAGAAGAAGAAGAAGAAGTTGAACATGAAG | ATGCACTGATCACGTGGCTGATCATGCACT |
| | (SEQ ID NO: 177) | (SEQ ID NO: 206) |
| | AATTTTAATACTAATAATGTTAATAATATG | CACACACACACACACACACACACACACACA |
| | (SEQ ID NO: 178) | (SEQ ID NO: 207) |
| | AATTTTAATACTAATAATGTTACTGATATG | CAGGAGGCTTGACTCCCTTGAGTCCACCCA |
| | (SEQ ID NO: 179) | (SEQ ID NO: 208) |
| | ACACTAAACACCATATATATTGAGAAAGAG | CATGATAGCCACGTGATCAGTGCATGATCA |
| | (SEQ ID NO: 180) | (SEQ ID NO: 209) |
| | ACCATATATATTGAGAAAGAGAGGTGAGAA | CCTCAGGAGGCTTGACTCCCTTGAGTCCAC |
| | (SEQ ID NO: 181) | (SEQ ID NO: 210) |
| | AGATAAATTTAAAGAAGTTAAAGAAGAACA | CCTCTCTCCAAACGCCTCAGGAGGCTTGAC |
| | (SEQ ID NO: 182) | (SEQ ID NO: 211) |
| | AGGCCTATGAAACCCACCGCAGTCAAGAAG | CGGCTCGGCTCGGCTCGGCTCGGCTCGGCT |
| | (SEQ ID NO: 183) | (SEQ ID NO: 212) |
| | ATAAATGGGGATTGGGGATTGGGGATTGGG | CGTGATCAGTGCATGATAGCCACGTGATCA |
| | (SEQ ID NO: 184) | (SEQ ID NO: 213) |
| | ATCTAATTTTAATACTAATAATGTTAATAA | CTCCAAACGCCTCAGGAGGCTTGACTCCCT |
| | (SEQ ID NO: 185) | (SEQ ID NO: 214) |
| | ATGCGTCATGCGTCATGCGTCATGCGTCAT | CTGATCATGCACTGATCACGTGGCTATCAT |
| | (SEQ ID NO: 186) | (SEQ ID NO: 215) |
| | ATGGGGATTGGGGATTGGGGATTGGGGATT | GCGACCTCTCTCCAAACGCCTCAGGAGGCT |
| | (SEQ ID NO: 187) | (SEQ ID NO: 216) |
| | ATTATTATTATTATTATTATTATTATTATT | GTCCACCCAGTGAGCTCCAAGAGATACCCG |
| | (SEQ ID NO: 188) | (SEQ ID NO: 217) |
| | CCAATCCCCAATCCCCAATCCCCAATCCCC | TCTTATACACATCTCGAGCCCACGAGACTC |
| | (SEQ ID NO: 189) | (SEQ ID NO: 218) |
| | CGTCATGCGTCATGCGTCATGCGTCATGCG | |
| | (SEQ ID NO: 190) | |
| | CTCTGCTCTGCTCTGCTCTGCTCTGCTCTG | |
| | (SEQ ID NO: 191) | |
| | CTCTTATACACATCTCGAGCCCACGAGACG | |
| | (SEQ ID NO: 192) | |
| | CTTATACACATCTCGAGCCCACGAGACAAC | |
| | (SEQ ID NO: 193) | |
| | CTTATACACATCTCGAGCCCACGAGACTGT | |
| | (SEQ ID NO: 194) | |
| | CTTTAAATTTATCTAATTTTAATACTAATA | |
| | (SEQ ID NO: 195) | |
| | CTTTAACTTCTTTAAATTTATCTAATTTTA | |
| | (SEQ ID NO: 196) | |
| | GATTGGGGATTGGGGATTGGGGATTGGGGA | |
| | (SEQ ID NO: 197) | |
| | TTTATCTAATTTTAATACTAATAATGTTAA | |
| | (SEQ ID NO: 198) | |

TABLE 4
Sequences from FIGS. 7 & 8

| | |
|---|---|
| FIG. 7 | AACCTGGTGGAAACCCCGAGCCTACAACAG (SEQ ID NO: 219) |
| | AAGTGTGGGTATACGCCCCATAGTGGGCGC (SEQ ID NO: 220) |
| | ACTTATCAAGCTTAACTGGCTAGTGATATA (SEQ ID NO: 221) |
| | AGCCCGCCTTGCAGCAGAAGCGCCACTGAG (SEQ ID NO: 222) |
| | ATTTCCAAGAACGGACCTCGCACATAACCG (SEQ ID NO: 223) |
| | CACTAAAAAGTAAAGTCGATCAGCCTGAAT (SEQ ID NO: 224) |
| | CATTGTACAACGAGGATGAAGCGTTACACA (SEQ ID NO: 225) |
| | CCCCCTTGTTGGTGCAATTACCATAGTAAA (SEQ ID NO: 226) |
| | CCGCAAAGCGGACTCGTAGCGATGGAAATG (SEQ ID NO: 227) |
| | CGGAGAGAACTACTTCCGTATTTTACCGCA (SEQ ID NO: 228) |
| | CGGGATGCATACATCGCCTACGGTAAGCAC (SEQ ID NO: 229) |
| | CGTGCAGGGTGCTGGAAGAGCACTTAAAGC (SEQ ID NO: 230) |
| | CTCGGCCCGTACAGGCACGGGGCACCAGTG (SEQ ID NO: 231) |
| | CTGAAGCAATACAGGCGTGTGACCGCGTAA (SEQ ID NO: 232) |
| | CTGACACCGCCCGTGACAATGAGGGTCCTG (SEQ ID NO: 233) |
| | GAGCCACCTAGTTAATAAATCCCTCTCACC (SEQ ID NO: 234) |
| | GAGGGAGCCCATATATCCTTGTTTCTTATA (SEQ ID NO: 235) |
| | GCGGGAGTTTCGGACGGATGTGGTCCCCTA (SEQ ID NO: 236) |
| | GCGGTGGGAAGGGCCGAAGACGAATTAGAA (SEQ ID NO: 237) |
| | GCTAGGTCATAGAGTTACATGCTCACCGGC (SEQ ID NO: 238) |
| | GGCTCGATGGTCTGGCTCATGGGTTCATCA (SEQ ID NO: 239) |
| | GGTCTCCAAAACTCCAAGTAGTCTACCCAC (SEQ ID NO: 240) |
| | GTAGCGAGTCTACGTTGCTCTGTCGATGCG (SEQ ID NO: 241) |
| | TAGATTCTAAACATCTTACGGCGATTTTCT (SEQ ID NO: 242) |
| | TCCCAGAGTTCTGTGGGGCTTTTTGTAAAA (SEQ ID NO: 243) |
| | TCGACCTCGTATGGTTCTCGGCAGCTGCGA (SEQ ID NO: 244) |
| | TCGGTAAATAAGGCCCCCATGATAAGTACT (SEQ ID NO: 245) |
| | TCGTGCAGCCACTACCGACTTAGGGGCCTG (SEQ ID NO: 246) |
| | TGGTCATCGCCTACACGTTAGCAGTCAACA (SEQ ID NO: 247) |
| | TTCCATGAGGTGGCTAGACGTTCCGCAAAT (SEQ ID NO: 248) |
| | TTGAAATACCGAGGCGTGTGGGTAAGTGAT (SEQ ID NO: 249) |
| | TTGTCCTAGCCATTATCTCGAAAAGGTCTT (SEQ ID NO: 250) |
| FIG. 8 | AAAGTCTGTCCGACTTGAATCCCTGTTGTT (SEQ ID NO: 251) |
| | ACAATCCCCAATCCCCAATCCCCATTTAAT (SEQ ID NO: 252) |
| | ACACACCTTGGCCAGCAACACACCACGCCA (SEQ ID NO: 253) |
| | ACACAGGACCGCCCTGCCAGTAGGTTTGCA (SEQ ID NO: 254) |
| | ACACAGGCAGCACCAGGAGATGCGGGCAGA (SEQ ID NO: 255) |
| | CCACATGGAAGAAGCGTCTCGGTATATAAT (SEQ ID NO: 256) |
| | CCACCACAATGGCGCCTTTCATTTTTGCCA (SEQ ID NO: 257) |
| | CCACCGCGCACGATGGTCTCCACGGGCCAT (SEQ ID NO: 258) |
| | CCACCTGTGTCGGTTTACGGTACGGGCTGC (SEQ ID NO: 259) |
| | CCACGTGCAGGTGAGATTATCCGTCAGGGA (SEQ ID NO: 260) |
| | CCACTCTTTAAATTGAGAATTGAAATTTGA (SEQ ID NO: 261) |
| | CCAGAGCCCATCAAGTATGTGGACAATCTG (SEQ ID NO: 262) |
| | CGAAGAGGTCGGCGGCCGAAGGGAAAGCCA (SEQ ID NO: 263) |
| | CTCCGAGCCCACCAGACCCGCGGTTCTATC (SEQ ID NO: 264) |
| | GAACGCCCATCTTGTCGAGGATGATACCTG (SEQ ID NO: 265) |
| | GAACGTCGAAGAGGTCGGCGGCCGAAGGGA (SEQ ID NO: 266) |
| | GACTCTCTAATTCGTTAATTCTCACTCTAG (SEQ ID NO: 267) |
| | GGATTCCCTGGCACAGGCCAGCGTGAGCCC (SEQ ID NO: 268) |
| | GGCCGTTACACAGATTTTCTTGCCTGTGGC (SEQ ID NO: 269) |
| | GGCTGTTGAGTGCGGTTACTGGCACCTGTG (SEQ ID NO: 270) |
| | GGTCCAAGGTAGGTCCAAGGTAGGTCCAAG (SEQ ID NO: 271) |
| | TGTGAAATTCCGCCAGAAAGGCGTGGAATA (SEQ ID NO: 272) |
| | TGTGATGAGATTGAGATGAAGGACAAGGAG (SEQ ID NO: 273) |
| | TTCCGTCCAGTCCTCGCCGCTCATCGCCTT (SEQ ID NO: 274) |
| | TTCGAACGTGAGCGAGCTCGATGTGAAACA (SEQ ID NO: 275) |

### Definitions

Below are definitions of groups and annotations used as examples in accordance with some embodiments of the invention:
F: The set of all farms participating in the study, F={f₁, f₂, . . . , f_{N}}.
F_{A}: The subset of farms selected for testing a specific additive A.
C_{u,i}: The set of “Learning Microbiome Cows” (LMCs) for each farm fᵢ ∈F, selected for unsupervised learning of the microbiome.
P_{X}: The collection of “microbial genetic patterns” derived from each microbiome sample X. Each sample gives rise to a distinct network. GX comprised of a constant set of M=|V| nodes and unique edges, E_{X}. Patterns may be extracted both from individual network analysis and from the superposition of networks, enabling exploration of a broad spectrum of combinatorial possibilities based on various criteria.
P_{S}: represent the patterns derived from a combination of networks, where S is the set of samples considered for superposition.
Ct,i and Cv,i: The partition of the LMCs into a “Microbiome Train Group” and a “Microbiome V_{\hat{d}} lidation Group,” respectively, for each farm fi∈F_{A}.
Cm,i: The (e.g., 40) cows selected for methane measurement in each farm fi∈F_{A}.
C_{mc,i}, C_{mt,i}, C_{mv,i}: The division of C_{m,i} into three groups “Control Methane Cows”, “Train Methane Cows” and “Test Methane Cows”.
Mₚᵣₑ(c) and Mₚₒₛₜ(c): The pre-additive and post-additive methane levels for a cow c. Since the cows were measured multiple times over the 12-week period following the introduction of the additive, multiple Mₚₒₛₜ(c) values may be obtained for each cow. This repeated sampling may bolster statistical confidence in the results.
T_{pre,i}, T_{post,i}, C_{pre,i}, C_{post,i}: The mean pre-additive and post-additive methane levels for the treatment and control cows in a farm fi.
η_{A,fi}: The methane efficacy for farm fi and additive A, calculated e.g. as:

$${η}_{A , fi} = \frac{{T}_{post , i} / {T}_{pre , i}}{{C}_{post , i} / {C}_{pre , i}} = \frac{{T}_{post , i} \cdot {C}_{pre , i}}{{T}_{pre , i} \cdot {C}_{post , i}}$$

Following are some notes regarding the groups and their properties:
The Learning Microbiome Cows (C_{u,i}) and the cows selected for methane measurement (C_{m,i}) in each farm may be disjoint, e.g., C_{u,i}∩C_{m,i}=Ø for each farm fi∈F_{A}.
The “Microbiome Train Group” (C_{t,i}) and “Microbiome V_{\hat{d}} lidation Group” (C_{v,i}) may also be disjoint, e.g., C_{t,i}∩C_{v,i}=Ø for each farm fi∈F_{A}.
Similarly, the “Control Methane Cows”, “Train Methane Cows” and “Test Methane Cows” groups may be pairwise disjoint for each farm fi∈F_{A}, i.e. C_{mc,I} ∩C_{mt,I}=Ø, C_{mc,I} ∩C_{mv,I}=Ø, and C_{mt,i}∩C_{mv,I}=0.
The efficacy of an additive A in a farm fi may be a time series measurement, e.g., represented as a set η_{A,fi}={e1, e2, . . . , en} where each ek may be calculated from the ratio of mean pre-additive and post-additive methane levels for the treatment and control cows.
The division of cows into “control methane cows” (CMC), “train methane cows” (TMC), and “test methane cows” (TeMC) has been optimized to minimize bias. This has been achieved by exhaustively examining all possible allocations of cows into the three groups, and selecting the assignment that minimizes the maximum discrepancy among the distributions of age (AGEᵢ), days in lactation (DILᵢ), and/or average milk yield (AMYᵢ) across the groups. Let Gr_{CMC}, Gr_{TMC}, and Gr_{TeMC} represent the groups of cows. The condition for the optimal assignment can be formally expressed e.g., as: For all i, and for any two distinct groups Grₓ and Gr_{y} from {Gr_{CMC}; Gr_{TMC}; Gr_{TeMC}} the assignment may be chosen that minimizes the following example quantity:

$$max \{ \begin{matrix}\vert P ( {AGE}_{i} \vert {Gr}_{x} ) - P ( {AGE}_{i} \vert {Gr}_{y} ) \vert , \\ \vert P ( {DIL}_{i} \vert {Gr}_{x} ) - P ( {DIL}_{i} \vert {Gr}_{y} ) \vert , \\ \vert P ( {AMY}_{i} \vert {Gr}_{x} ) - P ( {AMY}_{i} \vert {Gr}_{y} ) \vert\end{matrix} \}$$

This causes the selected division of cows into groups to have the least possible bias across the characteristics of age, days in lactation, and average milk yield among the cows. Other definitions, categorizations, and divisions of groups may be used.

### Training the Model

This stage may be executed per each feed additive A. Note that whereas not all farms are included in this stage (e.g., since feed additive A may have been tested by only a subset of the available farms), all of the data patterns extracted in the unsupervised phase may be used to train its efficacy prediction model. The subset of farms used for this stage may be denoted as F_{A} ⊆F.

For each farm fi∈F_{A}, the LMCs may be further partitioned into a “Microbiome Train Group” C_{t,i} ⊆C_{u,i} and a “Microbiome V_{\hat{d}} lidation Group” C_{v,i}=C_{u,i} \C_{t,i}. Also for each farm fi, a set of (e.g., 40) cows C_{m,i} may be measured for methane emissions. This set may be divided into e.g. three groups: “Control Methane Cows” C_{mc,i}, “Train Methane Cows” C_{mt,i} and “Test Methane Cows” C_{mv,i}. Mₚᵣₑ(c) and Mₚₒₛₜ(c) may denote the pre-additive and post-additive methane levels for a cow c, respectively. Mₚᵣₑ(c) and Mₚₒₛₜ(c) may be calculated as the median of methane levels over 30 to 120 seconds, e.g., excluding negligible values, such as, those smaller than 5 parts per million.

The methane efficacy η_{A,fi} for farm fi and additive A may be calculated e.g., as follows:

$${T}_{pre , i} = \text{?} {M}_{pre} ( c ) ,$$

$$\text{?} \text{indicates text missing or illegible when filed}$$

the means pre-additive methane for the treatment cows in the farm,

$${T}_{post , i} = \text{?} {M}_{post} ( c ) ,$$

$$\text{?} \text{indicates text missing or illegible when filed}$$

the means pre-additive for the treatment cows in the farm,

$${C}_{pre , i} = \text{?} {M}_{pre} ( c ) ,$$

$$\text{?} \text{indicates text missing or illegible when filed}$$

the means pre-additive methane for the control cows in the farm,

$${C}_{post , i} = \text{?} {M}_{post} ( c ) ,$$

$$\text{?} \text{indicates text missing or illegible when filed}$$

the means pre-additive methane for the control cows in the farm,
The overall efficacy may be calculated e.g., as:

$${η}_{A , fi} = \frac{{T}_{post , i} \cdot {C}_{pre , i}}{{T}_{pre , i} \cdot {C}_{post , i}}$$

This efficacy, along with the microbiome samples from Ct,i, may be used for the supervised learning process.

### Results

For each feed additive A and for each farm fi∈F_{A}, the microbiome samples from C_{v,i} were used to predict the additive's efficacy. This predicted efficacy was then contrasted with the actual efficacy determined through the analysis of methane emissions from Cmc,i and Cmv,i.

This design allows for general applicability to different additives and use cases, with potential for synergistic improvement as more data is added to the unsupervised learning stage. The division of cows into various groups may be performed in a manner that reduces or and/or minimizes bias, e.g., for factors such as age of cows, their days in lactation, and average milk yield.

Although some embodiments of the invention characterize improved biological conditions according to methane emissions reduction, such embodiments may also measure or correspond to a consequential increase in production yield that typically takes place when additives' efficacy is at its peak. In general, this yield enhancement is not merely a fortuitous result, but inextricably linked to methane reduction efforts. This relationship can be attributed to the metabolic energy redirection within the organism. As less energy is channeled towards methane production, typically more becomes accessible for other essential biological processes, such as milk production or body mass increase in cattle.

The correlation between methane emissions and yield has been well-documented in the literature. Studies have provided robust evidence substantiating this association. The prediction model according to embodiments of the invention, though described in some examples to effectively facilitate methane emissions reduction, may also be used to predict yield maximization. This demonstrates the dual environmental and economic benefits of this model, thereby contributing to the ongoing effort for sustainable farming practices.

### Microbial Data Analytics

Embodiments of the invention are predicated on the analysis of numerous microbiome samples collected from bovine subjects across diverse farm settings (e.g., ranging from different geographical locations, environmental conditions, herd sizes, and/or management practices). In experiments, a subset of these subjects have been administered a feed additive, and subsequent methane emissions were measured, creating an experimental group, while others remained as a control group. Each sample encapsulates a plethora of” reads,” each representing sequences of e.g., 100 to 150 nucleotides. Embodiments of the invention aim to identify significant microbial genetic patterns pertinent to a biological condition or trait of interest, e.g., the high efficacy of the feed additive.

A “k-mer” is a contiguous subsequence of length k derived from a longer string of nucleotides. In the context of genomics, a k-mer typically refers to a sequence of k nucleotides within a larger DNA or RNA sequence.

In a formal mathematical description, an original longer sequence of nucleotides may be denoted as the string S and its length as n, and a k-mer may be a substring of S of length k. Given S[i:j] that denotes the substring of S starting at position land ending at position j (e.g., inclusive), a k-mer of S starting at position i may be denoted as S[i: i+k−1]. Note that the starting position i may satisfy 1≤i≤n−k+1 to ensure the substring of length k can be obtained from S. Consequently, the total number of distinct k-mers that can be extracted from a sequence S of length n is n−k+1.

Furthermore, considering the biological context where each position in the string can be one of four nucleotides (e.g., A, T, C, or G in DNA or A, C, G, or U in RNA), the total number of possible k-mers of length k, without considering any specific longer sequence, is 4^{k}.

Embodiments of the invention may be characterized by an unbiased exploration of large k-mers, e.g. those with k=30, though not confined to this value. Previous studies have illustrated the optimal expressivity of k-mers of length 30 or longer for predictive applications. However, their use is typically constrained to cases of extreme data sampling or pre-set filtering criteria, both of which can introduce bias. Conversely, models that leverage k-mers as features in machine learning typically limit k to values of 6 or less, driven by concerns of data scarcity and potential model overfitting. Traditionally, an unbiased analysis of longer k-mers would be considered computationally impractical due to the vast number of possible combinations, approximately 2⁶⁰. Additionally, it would necessitate significant amounts of data to circumvent overfitting.

Embodiments of the invention leverage the understanding that the distribution of k-mers within DNA does not typically follow a uniform pattern but instead conforms to a power-law. This property allows embodiments of the invention to implement efficient analytic techniques and extract a significant number of k-mer groups automatically. Each of these groups may be verified as likely associated with a particular biological condition or epigenetic trait. However, the relevancy of such traits to our current interest may vary.

Embodiments of the invention may manifest in a dual capacity. Firstly, some embodiments extend our analysis beyond merely long k-mers, thereby enhancing their expressivity, to encompass groups of k-mers, which, inturn, fortifies their role as potent predictive features. Secondly, some embodiments address data paucity by utilizing a technique that capitalizes on the power-law distributed data property, as opposed to a brute force examination of “all k-mers” or “all groups.”

This technique facilitates the efficient detection of “correlated anomalies” localized groups giving rise to network structures which do not naturally arise in power-law networks. Analytically, the presence of such groups is indicative of an underlying causality within the data, signifying an association with a specific property relevant to the group of genetic information.

Additionally, the nature of this approach, based on the holistic examination of microbiome samples, allows for each group of k-mers to potentially comprise DNA fragments derived from heterogeneous sources. This denotes that functionalities emanating from diverse microbes may concurrently contribute to the observed behavior of interest.

### Workflow

The following workflow describes an embodiment of the invention for analyzing the microbial data:
1. Represent each raw sequenced microbial data set as a network, e.g., with a fixed number of nodes M, but with varying configurations of edges.
2. Create superposition networks by overlaying networks according to certain categories (e.g., samples from the same farm).
3. Perform an unsupervised analysis of each of the original one-sample networks and the superposition networks. Foreach network, execute the following steps:
   a. For each unique degree d in the network, examine all nodes with degree d.
   b. Empirically calculate the “internal connectivity” of this group of nodes (e.g., the ratio between the number of edges starting and ending at nodes within this group and the number of edges starting in this group and ending outside of it).
   c. If this ratio exceeds an anomaly criterion (e.g., set by Theorem 5.8), analytically determine that this group of nodes (e.g., k-mers) is likely to be statistically significant with some statistical confidence ϵ (e.g., a selected and/or tunable parameter).
4. Store all detected groups of k-mers for later use in the supervised phase. Each group has its own statistical confidence level which can be used for further analysis. However, all groups are guaranteed to be more robust than the initial E selected.

### Data Representation

As previously defined, from each farm fi∈F a random set of cows C_{u,i} are selected, and their microbiome sampled and sequenced.

Each microbiome sample, denoted as X, may contribute to the generation of a unique network, G_{X}, comprising M nodes and E_{X} edges. The nodes' consistency across all networks may originate from the initial data processing: during an automated preliminary analysis of the data, infrequently appearing k-mers may be excluded based on a filtering criterion (e.g., only k-mers that appear at least twice in at least two samples are retained; or a stricter criterion may be used to further reduce the number of k-mers). A manageable quantity M of k-mers remain, e.g., that is conducive to our network analytics. This provision is facilitated by the power-law distribution of k-mer repetitions across the data, which guarantees that the majority of k-mers will indeed be unique and subsequently filtered out, while predicting that there will still be a substantial number of repeating k-mers recurring multiple times.

In one example, k-mers may be 30-mers. The number k=30 is large enough to allow for sufficient expressivity of the k-mers but low enough to allow different k-mers to appear in the same read, subsequently manifested as an edge in the k-mers network. From a search space of 4³⁰ possible 30-mers, there are approximately one million 30-mers, translating into networks ofone million nodes, a scale that is computationally feasible to handle.

Note that changing the value of k trades the maximum number of possible k-mers for the maximum number of possible connections between them. V_{\hat{d}} rious values from k=20 to k=80 could be used with similar attributes, potentially producing DNA patterns that would further enhance the model's predictive capabilities.

An enhanced model that repeats the flow described here for various values of k, each resulting in a different set of k-mers, can still be managed computationally efficiently. Even a model with k-mers for 60 different values of k would result in networks of around 100 million nodes. Although such networks may initially seem too large, due to the efficient nature of embodiments of the invention, these networks can still be analyzed with practical computational resources.

Network Generation from K-Mers

The generation of each network, denoted as G_{X}, may involve a combination of genomic sequencing and preliminary data analysis, subsequently processed by an Artificial Intelligence (AI) engine (e.g., processor(s) 112 of FIG. 1). In the initial stage, the microbial DNA is fragmented into a set of k-mers (e.g., by processor(s) 108 of FIG. 1), e.g., 30-mers, each of which serves as a potential node in the network produced in the next phase. This process results in a theoretical search space of 4³⁰ k-mers.

To improve the efficiency of the network analysis, a preliminary filtering step may be used. Foreach microbial sample, only those k-mers that repeat a predefined number of times (e.g., at least twice) in the same sample are retained, and the rest are eliminated, thereby significantly reducing the search space. The use of this filtering is feasible due to the fact that the repetition of k-mers across the data follows a power-law distribution, allowing us to significantly reduce the size of the k-mer search space (e.g., as most of the k-mers have a unique appearance throughout each sample), while still statistically guaranteeing the existence of a large enough selection of k-mers that appear multiple times in each sample. This method results in approximately one million unique 30-mers that form the consistent nodes V used across all networks.

When new samples are received, they can be easily processed such that only k-mers from V are used for their analysis. From time to time, the nodes V can be refreshed by rerunning this process, expanding the set of supported k-mers and updating the internal ordering of the networks, allowing for new k-mers originating from new data to be included in the overall analysis. This process, however, is not mandatory, ensuring flexibility in our analytical approach.

Once the initial construction of the set of supported k-mers is completed, each microbiome sample X can be analyzed and its corresponding network G_{X}=G(V, E_{X}) can be constructed, e.g., as follows:
1. The sample X may contain a large number of “reads” (e.g., between 1 and 10 million(s)), each a string of nucleotides of length 100 to 150 bases.
2. For each read, extract all k-mers that are contained in the read and filter this list of k-mers per the list of supported k-mers V.
3. For each pair v₁ and v₂ of supported k-mers that are contained in this read, add an edge (v₁, v₂) to E_{X}.

Note that whereas each network G_{X} has its unique set of edges E_{X}, the nodes in the network may be the same group V, e.g., established during the initial filtering phase, using the multitude of available microbiome samples. In addition, the adjacency matrix of the network G_{X} may represent only the existence of connections without storing their actual strengths or quantities. In other words, a Boolean representation can be used for E_{X}, further reducing time and space complexities. Alternatively, the strengths or quantities of the connections may be stored and modeled.

Once the networks G_{X} are constructed from single microbial samples, further networks GS can be constructed on demand, for various groups S, by easily applying a logical OR operator on the adjacency matrices corresponding to the networks G_{X} for every X_{S}. Each of these networks can then be used as input for the network anomalies detection phase, allowing for the discovery of new data patterns, observable through the data-perspective provided by the semantics of the group S.

### Detection of Local Network Anomalies in the K-Mer Networks

Given a network G_{X} (V, E_{X}) (or G_{S}(V, E_{S})), the degrees of its nodes closely follows a power-law distribution: P (k) k^{-α}. Literature shows that in many real-world networks that follows a power-law degree distribution this pattern applies degrees larger than some minimal degree dmin. Namely, for some normalization constant c, the probability of a node v to have degree d≥dₘᵢₙ equals, e.g.:

$$∀ v ∈ V , ∀ d \geq {d}_{min} , P [ deg ( v ) = d ] = c \cdot {d}^{- α}$$

The expected value may first be calculated for the normalization constant c using values obtainable from the data:

Definition 5.1. Let V* denote all nodes of degree at least dmin and let E* denote the edges that have at least one side in V*, e.g.:

$${V}^{*} = \{ ( v ∈ V ) \vert ( deg ( v ) \geq {d}_{min} ) \}$$

$${E}^{*} = \{ ( ( v , u ) ∈ E ) \vert ( v ∈ {V}^{*} ) V ( u ∈ {V}^{*} ) \}$$

### Lemma 5.2. If:

$$∀ v ∈ V , ∀ d \geq {d}_{min} , P [ deg ( v ) = d ] = c \cdot {d}^{- α}$$

then

$$c = \frac{2 \vert {E}_{*} \vert}{\vert {V}_{*} \vert \cdot {\sum }_{d = {d}_{min}}^{{d}_{max}} {d}^{1 - α}}$$

Proof: The sum of a graph's G(V*, E*) degrees equals twice the number of its edges:

$${\sum }_{v ∈ {V}_{*}} deg ( v ) = 2 \vert {E}_{*} \vert$$

Taking into account the power-law distribution of the graph's nodes, and summing the expected number of nodes with degree d for every degree d from dmin to the maximal degree dmax implies that, e.g.:

$$2 \vert {E}_{*} \vert = {\sum }_{v ∈ {V}_{*}} deg ( v ) = E [ {\sum }_{d = {d}_{min}}^{{d}_{max}} d \cdot \vert ( v ∈ {V}_{*} ) ∧ ( deg ( v ) = d ) \vert ] = {\sum }_{d = {d}_{min}}^{{d}_{max}} d \cdot \vert {V}_{*} \vert \cdot P ( v ∈ {V}_{*} , deg ( v ) = d ) = {\sum }_{d = {d}_{min}}^{{d}_{max}} d \cdot \vert {V}_{*} \vert \cdot c \cdot {d}^{- α} = 2 \vert {E}_{*} \vert$$

Implying:

$$c = \frac{2 \vert {E}_{*} \vert}{\vert {V}_{*} \vert \cdot {\sum }_{d = {d}_{min}}^{{d}_{max}} {d}^{1 - α}}$$

From this, the expected number of nodes may be calculated having some degree d in a network G with a power-law degree distribution:

Definition 5.3. Let V_{\hat{d}} ⊆V* denote all nodes of degree \hat{d}≥dmin:

$${V}_{\hat{d}}^{{Δ}_{=}} \vert ( v ∈ {V}_{*} ) ∧ ( deg ( v ) = \hat{d} ) \vert$$

Lemma 5.4. If:

$$∀ v ∈ V , ∀ d \geq {d}_{min} , P [ deg ( v ) = d ] = c \cdot {d}^{- α}$$

then, for every degree dₘᵢₙ≤\hat{d}≤dₘₐₓ:

$$\vert {V}_{\hat{d}} \vert \approx \frac{2 \vert {E}_{*} \vert \cdot {\hat{d}}^{- α}}{\frac{1}{2 - α} ( {d}_{max}^{2 - α} - {d}_{min}^{2 - α} )}$$

Proof. The number of nodes with degree \hat{d} is:

$$\vert {V}_{\hat{d}} \vert = c \cdot \vert {V}_{*} \vert \cdot {\hat{d}}^{- α}$$

which using Lemma 5.2 equals:

$$\vert {V}_{\hat{d}} \vert = \vert {V}_{*} \vert \cdot {\hat{d}}^{- α} \cdot \frac{2 \vert {E}_{*} \vert}{\vert {V}_{*} \vert \cdot {\sum }_{d = {d}_{min}}^{{d}_{max}} {d}^{1 - α}}$$

Namely:

$$\vert {V}_{\hat{d}} \vert = \frac{2 \vert {E}_{*} \vert \cdot {\hat{d}}^{- α}}{{\sum }_{d = {d}_{min}}^{{d}_{max}} {d}^{1 - α}}$$

The denominator is a sum that goes, e.g., from dₘᵢₙ to dₘₐₓ. Since dₘₐₓ is large, this sum may be approximated, e.g., by the integral:

$${\sum }_{d = {d}_{min}}^{{d}_{max}} {d}^{1 - α} \approx {\int }_{{d}_{min}}^{{d}_{max}} {x}^{1 - α} dx$$

The integral can be evaluated by using the power rule for integration, e.g. as:

$${{\int }_{{d}_{min}}^{{d}_{max}} {x}^{1 - α} dx = \frac{{x}^{2 - α}}{2 - α} \vert}_{{d}_{min}}^{{d}_{max}} = \frac{{d}_{max}^{2 - α} - 1}{2 - α} - \frac{{d}_{min}^{2 - α} - 1}{2 - α} = \frac{{d}_{max}^{2 - α} - {d}_{min}^{2 - α}}{2 - α}$$

Substituting this back into the expression for |V_{\hat{d}}| gives, e.g.:

$$\vert {V}_{d} \vert \approx \frac{2 \vert {E}_{*} \vert \cdot {d}^{- α}}{\frac{1}{2 - α} ( {d}_{max}^{2 - α} - {d}_{min}^{2 - α} )}$$

This approximation holds, e.g., for α>2, where the integral is convergent.

Definition 5.5. Let λ denote the power-law normalizing constant for the graph G(V*, E*), e.g. as:

$$λ = \frac{2 ( 2 - α ) \vert {E}_{*} \vert}{( {d}_{max}^{2 - α} - {d}_{min}^{2 - α} )}$$

such that Lemma 5.4 can be written, e.g., as:

$$\vert {V}_{\hat{d}} \vert \approx λ {\hat{d}}^{- α}$$

Definition 5.6. Let E_{\hat{d}}⊆E denote all the edges that touch at least one node in V_{\hat{d}}. These edges may be divided into two complementing and mutually exclusive groups “internal edges”

$${E}_{\hat{d}}^{in}$$

(edges starting and ending in V_{\hat{d}}) and “external edges”

$${E}_{\hat{d}}^{out}$$

(edges that have one side in V_{\hat{d}} and one side in V\V_{\hat{d}}), e.g.:

$${E}_{\hat{d}} {=}^{△} \{ ( {v}_{1} , {v}_{2} ) \vert ( {v}_{1} ∈ {V}_{\hat{d}} ) ∨ ( {v}_{2} ∈ {V}_{\hat{d}} ) \}$$

$${E}_{\hat{d}}^{in} {=}^{△} \{ ( {v}_{1} , {v}_{2} ) \vert ( {v}_{1} ∈ {V}_{\hat{d}} ) ∧ ( {v}_{2} ∈ {V}_{\hat{d}} ) \}$$

$${E}_{\hat{d}}^{in} {=}^{△} \frac{{E}_{\hat{d}}}{{E}_{\hat{d}}^{in}}$$

Note that for every value of d the values of

$$\vert {E}_{\hat{d}}^{in} \vert$$

$$and$$

$$\vert {E}_{\hat{d}}^{out} \vert$$

can be acquired efficiently by counting edges in the network. The ratio between the number of “internal edges” and the number of “overall edges” can therefore be calculated for \hat{d}, which may represent the internal connectivity for degree \hat{d}.

Definition 5.7. Let β_{\hat{d}} denote the internal connectivity of the network G for the nodes of degree \hat{d}, e.g. as.

$${β}_{\hat{d}} {=}^{△} \frac{\vert {E}_{\hat{d}}^{in} \vert}{\vert {E}_{\hat{d}} \vert}$$

Since the number of edges in E_{\hat{d}} equals the sum of degrees of the nodes in V_{\hat{d}} minus the edges that has their two sides in V_{\hat{d}} (e.g., as these are counted twice), and since the degrees of the nodes in V_{\hat{d}} is exactly \hat{d}, then:

$${β}_{\hat{d}} = \frac{\vert {E}_{\hat{d}}^{in} \vert}{\vert {V}_{\hat{d}} \vert \cdot \hat{d} - \vert {E}_{\hat{d}}^{in} \vert}$$

Note that the value of β_{\hat{d}} can be efficiently calculated from the data for every value of \hat{d}. Some groups of nodes of the same degree may have high internal connectivity and some may have a lower one. It is therefore interesting to determine the expected value of the internal connectivity of various degrees, and a given internal connectivity value threshold above which would be considered “too strong” (e.g., representing a local network structure whose probability to spontaneously emerge in a network with power-law distribution of the degree is extremely low).

Theorem 5.8 provides an example anomaly criterion that for every degree \hat{d} greater than dₘᵢₙ can ensure that the set of nodes of degree \hat{d} are “too anomalous” with probability (1−ϵ) for every small threshold ϵ. This criterion applies for the internal connectivity of this group of nodes (e.g., the ratio between the number of edges connecting nodes of the same degree, and edges connecting nodes of different degrees), and may depend (only) on the degree \hat{d} and the network structural properties α, |E*|, dₘᵢₙ and dₘₐₓ.

Theorem 5.8. If:

$$∀ v ∈ V ,$$

$$∀ d ∈ {d}_{min} ,$$

$$P [ deg ( v ) = d ] = ϵ \cdot {d}^{- α}$$

then for any small threshold ϵ and any degree \hat{d}>dmin the group of nodes of degree \hat{d} with internal connectivity β\hat{d} may be considered “unlikely to spontaneously emerge” with respect to the threshold ϵ if e.g. the following is satisfied by β_{\hat{d}}:

$${β}_{\hat{d}} \geq \frac{{B}_{1} ( \sqrt{{B}_{2}^{2} - 4 {B}_{3}} - {B}_{2} )}{λ {\hat{d}}^{1 - α} - {B}_{1} ( \sqrt{{B}_{2}^{2} - 4 {B}_{3}} - {B}_{2} )}$$

$${B}_{1} = \frac{1}{2 + \frac{λ {\hat{d}}^{1 - α}}{\vert {E}_{*} \vert}}$$

$${B}_{2} = 1 - \frac{{λ}^{2} {\hat{d}}^{2 - 2 α}}{\vert {E}_{*} \vert} + {( {Φ}^{- 1} ( 1 - ϵ ) )}^{2} \frac{λ {\hat{d}}^{1 - α}}{2 \vert {E}_{*} \vert}$$

$${B}_{3} = {( 0.5 - \frac{{λ}^{2} {\hat{d}}^{2 - 2 α}}{\vert {E}_{*} \vert} )}^{2} - {( {Φ}^{- 1} ( 1 - ϵ ) )}^{2} \frac{{λ}^{2} {\hat{d}}^{2 - 2 α}}{2 \vert {E}_{*} \vert} ( 1 - \frac{λ {\hat{d}}^{1 - α}}{2 \vert {E}_{*} \vert} )$$

Proof. For \hat{d}≥dₘᵢₙ and for an edge e∈E_{\hat{d}}, by definition at least one of its sides is in V_{\hat{d}}. The probability that the second side of this edge also ends in V_{\hat{d}} as well is affected by e.g. two factors: first, the more nodes there are in V_{\hat{d}} the greater the chance that they would be selected for the edge e. Second, as the network has a power-law degree distribution, it may be assumed that it also follows the “preferential attachment” principle (e.g., that nodes with higher degree are more likely to acquire new connections, leading to a skew in the distribution). Under this principle, the probability of a node being chosen for a new connection is proportional to its degree, and the probability for the edge e to have both sides in V_{\hat{d}} is therefore a Bernoulli trial with success probability, e.g.:

$$p = \frac{d \cdot \vert {V}_{\hat{d}} \vert}{{\sum }_{d - {d}_{min}}^{{d}_{max}} ( d \cdot \vert {V}_{\hat{d}} \vert )} = \frac{d \cdot \vert {V}_{\hat{d}} \vert}{2 \vert {E}_{*} \vert} ;$$

$${E}_{\hat{d}}^{in} \sim Binomial ( \vert {E}_{\hat{d}} \vert \cdot \frac{\hat{d} \cdot \vert {V}_{\hat{d}} \vert}{2 \vert {E}_{*} \vert} )$$

A value of β_{\hat{d}} that is “too high” (e.g., unlikely to occur with a probability less than some threshold ϵ) may indicate that the number of successes from E_{\hat{d}} trials is greater than some threshold xₜₕᵣₑₛ.

To calculate an upper threshold for the number of successes in Bernoulli trials that is considered statistically significant at level ϵ, and since the number of trials N is large, and the success probability p is not too close to 0 or 1, the Binomial distribution may be approximated by a Normal distribution. If X~Binomial(N,p), it can be approximated as X≈Y where Y~Normal(μ, σ²) with μ=Np and σ²=Np(1−p), e.g.:

$$X \sim Normal ( Np , Np ( 1 - p ) )$$

This approximation allows us to express the cumulative distribution function (CDF) of X in terms of the CDF of the Normal distribution Φ, e.g., as follows:

$$P ( X \leq x ) \approx Φ ( \frac{x + 0.5 - Np}{\sqrt{Np ( 1 - p )}} )$$

Having Φ(x) formally defined as:

$$Φ ( x ) = \frac{1}{\sqrt{2 π}} {\int }_{- ∞}^{x} {e}^{\text{?}} dt$$

$$\text{?} \text{indicates text missing or illegible when filed}$$

To find a threshold xₜₕᵣₑₛ such that the probability of observing more than xₜₕᵣₑₛ successes is less than some small ϵ, the Normal approximation may be used to give, e.g.:

$$1 - Φ ( \frac{{x}_{thres} + 0.5 - Np}{\sqrt{Np ( 1 - p )}} ) < ϵ$$

Or equivalently,

$${Φ}^{- 1} ( 1 - ϵ ) = \frac{{x}_{thres} + 0.5 - Np}{\sqrt{Np ( 1 - p )}}$$

Solving for xₜₕᵣₑₛ gives:

$${x}_{\text{?}} = {Φ}^{- 1} ( 1 - ϵ ) \sqrt{Np ( 1 - p )} + Np - 0.5$$

$$\text{?} \text{indicates text missing or illegible when filed}$$

This provides the threshold value of the number of successes for which the probability of achieving that number or more is less than ϵ.

Using the original expressions for N and p this means that the number of internal edges above which a group of nodes with a given degree is considered “unlikely” is, e.g.:

$${x}_{thres} = {Φ}^{- 1} ( 1 - ϵ ) \sqrt{\vert {E}_{\text{?}} \vert \frac{\hat{d} \vert {V}_{\text{?}} \vert}{2 \vert {E}_{\text{?}} \vert} ( 1 - \frac{\hat{d} \vert {V}_{\text{?}} \vert}{2 \vert {E}_{\text{?}} \vert} )} + \vert {E}_{\text{?}} \vert \frac{\hat{d} \vert {V}_{\text{?}} \vert}{2 \vert {E}_{\text{?}} \vert} - 0.5$$

$$\text{?} \text{indicates text missing or illegible when filed}$$

Since we already know that:

$$\vert {E}_{\text{?}} \vert = \vert {V}_{\text{?}} \vert \cdot \hat{d} - \vert {E}_{\text{?}}^{\text{?}} \vert$$

$$\text{?} \text{indicates text missing or illegible when filed}$$

This means e.g., that:

$${x}_{\text{?}} = {Φ}^{- 1} ( 1 - ϵ ) \times \sqrt{( \vert {V}_{\text{?}} \vert \cdot \hat{d} - {x}_{\text{?}} ) \frac{\hat{d} \vert {V}_{\text{?}} \vert}{2 \vert {E}_{\text{?}} \vert} ( 1 - \frac{\hat{d} \vert {V}_{\text{?}} \vert}{2 \vert {E}_{\text{?}} \vert} )} + ( \vert {V}_{\text{?}} \vert {d}^{\text{?}} - {x}_{\text{?}} ) \frac{\hat{d} \vert {V}_{\text{?}} \vert}{2 \vert {E}_{\text{?}} \vert} - 0.5$$

$$\text{?} \text{indicates text missing or illegible when filed}$$

Rearranging:

$${x}_{\text{?}} ( 1 + \frac{\hat{d} \vert {V}_{\text{?}} \vert}{2 \vert {E}_{\text{?}} \vert} ) + 0.5 - \frac{{\hat{d}}^{2} \vert {V}_{\text{?}}^{2} \vert}{2 \vert {E}_{\text{?}} \vert} = {Φ}^{- 1} ( 1 - ϵ ) \sqrt{( \vert {V}_{\text{?}} \vert \hat{d} - {x}_{\text{?}} ) \frac{\hat{d} \vert {V}_{\text{?}} \vert}{2 \vert {E}_{\text{?}} \vert} ( 1 - \frac{\hat{d} \vert {V}_{\text{?}} \vert}{2 \vert {E}_{\text{?}} \vert} )}$$

$$\text{?} \text{indicates text missing or illegible when filed}$$

Squaring both sides:

where, e.g.:

$${A}_{1} {x}_{\text{?}}^{2} + {A}_{2} {x}_{thres} + {A}_{3} = 0$$

$$\text{?} \text{indicates text missing or illegible when filed}$$

$$\text{?} \\ {A}_{1} = {( 1 + \frac{\hat{d} \vert {V}_{\text{?}} \vert}{2 \vert {E}_{\text{?}} \vert} )}^{2}$$

$${A}_{2} = ( 1 + \frac{\hat{d} \vert {V}_{\text{?}} \vert}{2 \vert {E}_{\text{?}} \vert} ) \times ( 1 - \frac{{\hat{d}}^{2} {\vert {V}_{\text{?}} \vert}^{2}}{2 \vert {E}_{\text{?}} \vert} + {( {Φ}^{- 1} ( 1 - ϵ ) )}^{2} \frac{\hat{d} \vert {V}_{\text{?}} \vert}{2 \vert {E}_{\text{?}} \vert} )$$

$${A}_{3} = {( 0.5 - \frac{{\hat{d}}^{2} {\vert {V}_{\text{?}} \vert}^{2}}{2 \vert {E}_{\text{?}} \vert} )}^{2} - {( {Φ}^{- 1} ( 1 - ϵ ) )}^{2} \frac{{\hat{d}}^{2} {\vert {V}_{\text{?}} \vert}^{2}}{2 \vert {E}_{\text{?}} \vert} ( 1 - \frac{\hat{d} \vert {V}_{\text{?}} \vert}{2 \vert {E}_{\text{?}} \vert} )$$

$$\text{?} \text{indicates text missing or illegible when filed}$$

Solving this quadratic equation, and noting that A₁>0, yields, e.g.:

$${x}_{\text{?}} = {B}_{1} ( \sqrt{{B}_{2}^{2} - 4 {B}_{3}} - {B}_{2} )$$

$${B}_{1} = \frac{1}{2 + \frac{\text{?}}{\text{?}}}$$

$${B}_{\text{?}} = 1 - \frac{{\hat{d}}^{2} {\vert {V}_{\text{?}} \vert}^{2}}{2 \vert {E}_{\text{?}} \vert} + {( {Φ}^{- 1} ( 1 - ϵ ) )}^{2} \frac{{\hat{d}}^{2} \vert {V}_{\text{?}} \vert}{2 \vert {E}_{\text{?}} \vert}$$

$${B}_{\text{?}} = {( 0.5 - \frac{{\hat{d}}^{2} {\vert {V}_{\text{?}} \vert}^{2}}{2 \vert {E}_{\text{?}} \vert} )}^{2} - {( {Φ}^{- 1} ( 1 - ϵ ) )}^{2} \frac{{\hat{d}}^{2} {\vert {V}_{\text{?}} \vert}^{2}}{2 \vert {E}_{\text{?}} \vert} ( 1 - \frac{{\hat{d}}^{2} \vert {V}_{\text{?}} \vert}{2 \vert {E}_{\text{?}} \vert} )$$

$$\text{?} \text{indicates text missing or illegible when filed}$$

Using Lemma 5.4 this can be written as, e.g.:

$${x}_{\text{?}} = {B}_{1} ( \sqrt{{B}_{2}^{2} - 4 {B}_{3}} - {B}_{2} )$$

$${B}_{1} = \frac{1}{2 + \frac{\text{?}}{\text{?}}}$$

$${B}_{\text{?}} = 1 - \frac{{λ}^{2} {\hat{d}}^{2 - 2 α}}{\vert {E}_{*} \vert} + {( {Φ}^{- 1} ( 1 - ϵ ) )}^{2} \frac{λ {\hat{d}}^{\text{?} - α}}{2 \vert {E}_{*} \vert}$$

$${B}_{} = {( 0.5 - \frac{{λ}^{2} {d}^{\text{?}}}{2 \vert {E}_{\text{?}} \vert} )}^{2} - {( {Φ}^{- 1} ( 1 - ϵ ) )}^{2} \frac{{λ}^{2} {d}^{\text{?}}}{2 \vert {E}_{\text{?}} \vert} ( 1 - \frac{λ {d}^{\text{?}}}{2 \vert {E}_{\text{?}} \vert} )$$

$$\text{?} \text{indicates text missing or illegible when filed}$$

Recalling Definition 5.7 this means that for a given degree \hat{d} a group of nodes would be considered “unlikely” for an internal connectivity greater than, e.g.:

$${β}_{\text{?}} \geq \frac{{x}_{thres}}{\vert {V}_{\text{?}} \vert \cdot \hat{d} - {x}_{thres}}$$

$$\text{?} \text{indicates text missing or illegible when filed}$$

Note that the memory and time complexity of this approach is remarkably low due to the aforementioned techniques, which may allow for regular retraining as new data is obtained. This low complexity, coupled with the utilization of large k for k-mers (e.g., k≥10, 20 or 30), enhances prediction accuracy significantly. The expressivity of large k-mers, which refers to their ability to encapsulate functionality, contributes to the robustness and precision of our analysis.

### Discussion of Livestock Host Applications

Embodiments of the invention may implement a novel methodology in livestock herd environments that utilizes the cow microbiome as a basis for predicting the efficacy of methane-reducing additives. The structure of the trial followed a two-step approach: initially, microbiome samples were collected from cattle across an array of farms. These farms were chosen to represent a broad spectrum of conditions, including geographical location, environmental context, herd size, and differing management methods. The genetic data obtained from these samples was then analyzed via unsupervised learning to discern meaningful microbial genetic patterns. In the subsequent stage, a selected subset of cows from these farms was subjected to methane emission measurements under both control and experimental conditions (the latter involving the feed additive). This stage utilized supervised learning, building upon the established microbial baseline from the first step.

The design of this process, combined with the unsupervised genetic material detection methodology, can be extended as a predictive framework for various other properties, given appropriate labeling for the supervised learning stage. Positive network effects were exhibited as more data are collected and incorporated into the unsupervised learning stage, the accuracy of the predictions improves. When new use-cases are introduced, they contribute additional microbiome samples, further improving the predictive capability across all use-cases.

The division of cows into different groups (e.g., “control methane cows”, “train methane cows”, and “test methane cows”) improved predictive accuracy. Sorting cows was performed randomly, but with meticulous attention to mitigating any potential biases. Several key factors were taken into account, including the age of cows, their days in lactation, and average milk yield. Using these factors to inform the random selection created groups that were representative of the general population of the herd. This ensured that the microbiome samples and methane measurements taken from each group were not skewed by any one particular attribute, enhancing the generalizability and applicability of this model.

Macroscopic Microbial Data Properties: This model reveals a striking and noteworthy pattern in the distribution of the repetition of the reads and the k-mers. Specifically, the expression of these repetitions follows a power-law distribution. This observation was observed to be consistent and sustained, evident in the data extracted from multiple cows, and across both read and k-mer repetitions.

The emergence of this power-law distribution was observed to be data agnostic, i.e., that it is not an artifact of specific data acquisition processes or specific to the type of data being studied. Indeed, the observed power-law distribution suggests the existence of underlying principles governing these distributions, which extend beyond the specifics of this application.

One might suggest that the shotgun sequencing method, which was employed to sequence the DNA samples, could somehow skew the data towards this power-law distribution. However, this is highly unlikely. While the shotgun sequencing method can indeed introduce certain biases into the data, there is no reason to suspect that it would give rise to a power-law distribution. This is because shotgun sequencing, by its nature, is a random process and, as such, is not predisposed towards creating any specific pattern, much less one as distinctive as a power-law distribution.

Moreover, when the data was pooled from several cows together and then analyzed the distribution of popularity, the power-law distribution was consistently observed. This further strengthens the assertion that the power-law distribution is not an artifact of data acquisition. If it were, the distribution would be different for each cow, reflecting the individual variances in data collection. The consistent manifestation of the power-law distribution across different cows suggests a more profound, universal principle at work.

Interestingly, the power-law distribution was observed in the repetition of k-mers, as well as whole reads. This consistency across different levels of granularity in the data adds another layer of validation to these findings. The emergence of a power-law distribution at different scales strongly suggests the existence of scale-free, fractal-like patterns in the data, which is a key characteristic of power-law distributions.

Taken together, this strongly suggest that the observed power-law distribution is not a mere artifact of specific methodology, but rather points to a fundamental biological phenomena that has profound implications for our understanding of microbiome DNA and the factors influencing the efficacy of methane-reducing additives.

### Variations:

While this methodology attempts to mitigate the potential for bias, with the division of cows into “control methane cows”, “train methane cows”, and “test methane cows” carefully executed to take into account factors such as the age of cows, their days in lactation, and average milk yield, other factors may also be taken into account (e.g., by introducing stratification or covariate balancing) to reduce biases based thereon. For example, individual differences in cow physiology, diet, and environmental factors could be incorporated to reduce their effect on both the composition of the microbiome and the methane production.

Additionally or alternatively, instead of no overlap between the cow groups from which microbiomes were sampled and those from which methane was measured (which may present another potential source of bias), the groups may in some embodiments overlap. While our results suggest that a small microbiome sample is representative of the entire herd's microbiome, this assumption may not hold in all situations. Variations in individual cow microbiomes within the same herd could potentially impact the effectiveness of the methane-reducing additives and thus should be sampled in both groups.

Additionally or alternatively, to improve the predictive framework the accuracy and volume of the labeled data during the supervised learning stage may be bolstered to improve the model's predictive capability. For example, multiple different sensor sources may be used to cross-validate labeled data.

Additionally or alternatively, our experiments measured the methane reduction potential of additives, but may be applied to measure any biological condition, including for example, other potential effects of these additive son cow health, productivity, and the quality of dairy products.

Impact: Experimental results were promising, demonstrating a correlation between certain microbiome patterns and the efficacy of methane-reducing additives (see e.g. FIG. 8). This allows us to predict the impact of additives based on a cow's microbiome, enhancing the average effect by using the additive only when it is expected to be effective.

The potential impact of these findings is substantial. Given the ongoing need to reduce methane emissions for environmental reasons, the ability to predict the effectiveness of methane-reducing additives at an individual cow level could lead to significant improvements in emission reduction strategies.

Moreover, even though methane measurements were only conducted at some farms, the broad-scale collection of microbiome data enriched the initial unsupervised learning phase, enhancing the detection of significant microbial genetic patterns. There was no overlap between the cow groups from which microbiomes were sampled and those from which methane was measured, affirming that a small microbiome sample is typically representative of the entire herd's microbiome.

This methodology may assume strong microbiome homogeneity across the cows within a single herd. This means that while the microbiome samples were collected from one group of cows, these samples are considered indicative of the microbiome composition of the entire herd. The validity of this assumption is confirmed when these microbiome samples are successfully used to predict the efficacy of the methane-reducing additives in a distinct group of cows within the same herd. This suggests that despite no overlap in the cows from which the microbiome and methane measurements were taken, the microbiome sample effectively represents the herd's overall microbiome. This may result in a technique that is both robust and scalable, as it allows for a comprehensive prediction model without requiring invasive and extensive sampling from every cow.

Embodiments of the invention establish a novel methodology for predicting the efficacy of methane-reducing additives, and a potential of using host microbiomes as a tool to predict other biologically significant outcomes based on microbiome composition, such as disease susceptibility or productivity measures like milk yield and growth rate. Such predictive models may aid in early intervention strategies and improve the overall health and productivity of any host, including animals or humans.

Experiments indicate that a small microbiome sample is representative of the entire herd's microbiome. This may be used for non-invasive, rapid and cost-effective microbiome sampling techniques that could be used at scale.

Given the reliance of model training on the availability and quality of data, embodiments of the invention may incorporate a continuous and broad-scale collection of microbiome and methane emission data, to further refine the predictive model, increase its accuracy, and extend its applicability across various hosts (e.g., different breeds of cows) and/or different geographical locations (e.g., worldwide).

Finally, while experiments focused primarily on the methane reduction potential of additives, embodiments of the invention extend to predicting other biological conditions or effects of these additives (e.g., any unintended consequences of their use, such as changes to cow health or milk quality) to understanding the holistic impact of these additives and provide a comprehensive view and aid in making informed decisions regarding their widespread use.

Overall, embodiments of the invention may be used to intelligently manage livestock or other animals, e.g., including personalized additive regimens informed by each cow's microbiome, optimizing both environmental impact and productivity outcomes.

### CONCLUSION

Some embodiments of the invention may use “features” for training the groups in the first unsupervised learning stage. Features may be each instance of an anomalous group of microbiome DNA sequences or k-mer nodes (or score thereof, e.g., how many times the group occurs) detected in hosts. Training may generate a correlation between the feature (e.g., group or score thereof) and a biological condition (e.g., additive efficacy). Some embodiments of the invention may select only above threshold correlative strength btw group (score) and biological condition.

Embodiments of the invention described in reference to cows or other livestock hosts may be used for any organism including humans. In some embodiments, hosts may be a single species, or a combination of multiple different species, within the same genus or not. For example, human and cow microbiome DNA may be analyzed together for co-occurrences across different species' DNA.

Hosts or donors of biological samples sequenced into the microbiome DNA sequences discussed herein are living organisms. One or more processor(s) may sequence the organism's microbiome DNA obtained from its biological sample to identify the genetic constituents of the DNA. The processor(s) used to sequence DNA may be the same processor(s) or different processor(s) (e.g., external service provider) used to analyze and model the data. In some embodiments, “shotgun” sequencing may be used (e.g., providing rich data, but with no semantic association to the microbes themselves).

While embodiments of the invention describe microbiome DNA, any DNA or RNA may be used, for example, sequenced from any biological host sample or specimen including, for example, bile, blood, tissue, saliva, stem-cells, tumors, biopsies, etc. DNA may be sampled from a single host sample or a DNA mixture sequenced from multiple host samples.

As used herein, a “DNA” may refer to Deoxyribonucleic acid and/or Ribonucleic acid (RNA), such as, messenger RNA (mRNA), transfer RNA (tRNA), and/or ribosomal RNA (rRNA), or any physical genetic material. RNA contains adenine (A), cytosine (C), guanine (G), or uracil (U) nucleotide bases, whereas DNA contains adenine (A), cytosine (C), guanine (G) and thymine (T). DNA sequence data structures may include, for example, one or more vectors, scalar values, functions, sequences, sets, matrices, tables, lists, arrays, and/or other data structures, representing biological genetic material including one or more bases, nucleotides, genes, alleles, codons or other generic material.

As used herein, “practical,” “possible,” or “finite” time, computations, problems or tasks may refer to those that can be executed with a standard computer system on the order of less than a few (e.g., one or two) hours or days, and “impractical” or “impossible” time, computations, problems or tasks may refer to those that can only be executed with a standard computer system on the order of greater than a few (e.g., one or two) hours or days.

As used herein, “only” may mean exclusively, almost exclusively such as having less than 1-10% exception or with a number of exceptions that is less than a small proportion thereof; occurring in greater than 1-10% of instances or with a number of instances that is greater than a dominant proportion thereof, predominantly, in a majority (≥50%) of instances.

In the foregoing description, various aspects of the present invention are described. For purposes of explanation, specific configurations and details are set forth in order to provide a thorough understanding of the present invention. However, it will also be apparent to persons of ordinary skill in the art that the present invention may be practiced without the specific details presented herein. Furthermore, well known features may be omitted or simplified in order not to obscure the present invention.

Unless specifically stated otherwise, it is appreciated that throughout the specification discussions utilizing terms such as “processing,” “computing,” “calculating,” “determining,” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the invention may include an article such as a computer or processor readable non-transitory storage medium, such as for example a memory, a disk drive, or a USB flash memory device (e.g., memory unit(s) 114 and/or 118 of FIG. 1) encoding, including or storing instructions, e.g., computer-executable instructions, which when executed by a processor or controller (e.g., controller(s) or processor(s) 108 and/or 112 of FIG. 1), cause the processor or controller to carry out methods disclosed herein.

Different embodiments are disclosed herein. Features of certain embodiments may be combined with features of other embodiments; thus certain embodiments may be combinations of features of multiple embodiments.

The foregoing description of the embodiments of the invention has been presented for the purposes of illustration and description. It is not intended to be exhaustive or to limit the invention to the precise form disclosed. It should be appreciated by persons of ordinary skill in the art that many modifications, variations, substitutions, changes, and equivalents are possible in light of the above teaching. For example, it should be appreciated that sign conventions are equivalent and that embodiments of the invention in which values are above a lower bound or threshold are equivalent to embodiments of the invention in which values are below an upper bound or threshold, since the difference is a mere convention of sign. It is, therefore, to be understood that the appended claims are intended to cover all such modifications and changes as fall within the true spirit of the invention.

1. A method for detecting biomarkers in microbiome DNA of one or more livestock hosts to predict a biological condition, the method comprising:
storing a plurality of microbiome DNA sequences, sequenced from microbiome DNA of one or more livestock hosts;
generating a microbiome network comprising a plurality of nodes representing the respective plurality of microbiome DNA sequences and a plurality of edges representing a co-occurrence of each pair of microbiome DNA sequences in a same sample or sub-length of the microbiome DNA;
bundling, into distinct groups, microbiome DNA sequences represented by nodes based on the node's degree, the degree of each node quantifying a number of edges that contain the node indicating a number of unique microbiome DNA sequences that co-occur with the microbiome DNA sequence represented by the node in the same sample or sub-length of the microbiome DNA;
validating groups of microbiome DNA sequences that each have an internal connectivity between nodes of the same group that satisfies an anomaly condition indicating the microbiome DNA sequences of that group co-occur with a probability that is unlikely randomly statistical;
generating a training dataset correlating the validated groups of microbiome DNA sequences from one or more livestock hosts with a biological condition measured in one or more livestock hosts; and
training a machine learning model with the training dataset to input microbiome DNA sequences from one or more livestock hosts and predict the biological condition for one or more livestock hosts.

2. The method of claim 1, wherein the internal connectivity is determined based on a ratio between a number of internal edges connecting two nodes internal to the same group and a total number of overall edges connecting nodes within the group to any other node internal or external to the group.

3. The method of claim 2, wherein the internal connectivity condition for a group of nodes having a same degree \hat{d} is that the group's internal connectivity is greater than or equal to:
$$\frac{{x}_{thres}}{\vert {V}_{\hat{d}} \vert \cdot \hat{d} - {x}_{thres}} ,$$
where xₜₕᵣₑₛ is a threshold in which the probability of observing more than the threshold is less than a value ϵ and |V_{\hat{d}}| is the number of nodes in the microbiome network of degree \hat{d}.

4. The method of claim 1, wherein the internal connectivity is determined based on a number of nodes in the same group, number of edges in the same group, a number of pre-defined partially or fully connected clusters in the same group or deviation thereof.

5. The method of claim 1, wherein the internal connectivity condition is satisfied if the internal connectivity for a group of nodes deviates from an expected internal connectivity in a network that follows a power law distribution.

6. The method of claim 1 comprising filtering the plurality of microbiome DNA sequences to include only microbiome DNA sequences that occur greater than a predefined integer number of times in the same sample or sub-length of the microbiome DNA.

7. The method of claim 1 comprising filtering the plurality of microbiome DNA sequences to include only nodes within a predefined degree range and edges connecting those nodes.

8. The method of claim 1 comprising super-positioning a plurality of networks each representing a distinct microbiome DNA sample to generate a composite network representing a plurality of microbiome DNA samples.

9. The method of claim 1 comprising retraining the model based on new microbiome DNA.

10. The method of claim 1, wherein the biological condition of the one or more livestock hosts is selected from the group consisting of: feed, additive or medicinal efficacy, methane emissions, dairy or meat quality, composition or yield, gastrointestinal or overall health, disease susceptibility, disease tolerance, likelihood of disease recovery, life expectancy, and/or fatality risk.

11. A system for detecting biomarkers in microbiome DNA of one or more livestock hosts to predict a biological condition, the system comprising:
one or more memories configured to store a plurality of microbiome DNA sequences, sequenced from microbiome DNA of one or more livestock hosts; and
one or more processors configured to:
generate a microbiome network comprising a plurality of nodes representing the respective plurality of microbiome DNA sequences and a plurality of edges representing a co-occurrence of each pair of microbiome DNA sequences in a same sample or sub-length of the microbiome DNA,
bundle, into distinct groups, microbiome DNA sequences represented by nodes based on the node's degree, the degree of each node quantifying a number of edges that contain the node indicating a number of unique microbiome DNA sequences that co-occur with the microbiome DNA sequence represented by the node in the same sample or sub-length of the microbiome DNA,
validate groups of microbiome DNA sequences that each have an internal connectivity between nodes of the same group that satisfies an anomaly condition indicating the microbiome DNA sequences of that group co-occur with a probability that is unlikely randomly statistical,
generate a training dataset correlating the validated groups of microbiome DNA sequences from one or more livestock hosts with a biological condition measured in one or more livestock hosts, and
train a machine learning model with the training dataset to input microbiome DNA sequences from one or more livestock hosts and predict the biological condition for one or more livestock hosts.

12. The system of claim 11, wherein the one or more processors are configured to determine the internal connectivity based on a ratio between a number of internal edges connecting two nodes internal to the same group and a total number of overall edges connecting nodes within the group to any other node internal or external to the group.

13. The system of claim 12, wherein the one or more processors are configured to determine that a group of nodes having a same degree \hat{d} satisfy the internal connectivity condition if the group's internal connectivity is greater than or equal to:
$$\frac{{x}_{thres}}{\vert {V}_{\hat{d}} \vert \cdot \hat{d} - {x}_{thres}} ,$$
where xₜₕᵣₑₛ is a threshold in which the probability of observing more than the threshold is less than a value ϵ and |V_{\hat{d}}| is the number of nodes in the microbiome network of degree \hat{d}.

14. The system of claim 11, wherein the one or more processors are configured to determine the internal connectivity based on a number of nodes in the same group, number of edges in the same group, a number of pre-defined partially or fully connected clusters in the same group or deviation thereof.

15. The system of claim 11, wherein the one or more processors are configured to determine that the internal connectivity condition is satisfied if the internal connectivity for a group of nodes deviates from an expected internal connectivity in a network that follows a power law distribution.

16. The system of claim 11, wherein the one or more processors are configured to filter the plurality of microbiome DNA sequences to include only microbiome DNA sequences that occur greater than a predefined integer number of times in the same sample or sub-length of the microbiome DNA.

17. The system of claim 11, wherein the one or more processors are configured to filter the plurality of microbiome DNA sequences to include only nodes within a predefined degree range and edges connecting those nodes.

18. The system of claim 11, wherein the one or more processors are configured to super-position a plurality of networks each representing a distinct microbiome DNA sample to generate a composite network representing a plurality of microbiome DNA samples.

19. The system of claim 11, wherein the one or more processors are configured to retrain the model based on new microbiome DNA.

20. The system of claim 11, wherein the biological condition of the one or more livestock hosts is selected from the group consisting of: feed, additive or medicinal efficacy, methane emissions, dairy or meat quality, composition or yield, gastrointestinal or overall health, disease susceptibility, disease tolerance, likelihood of disease recovery, life expectancy, and fatality risk.


SEQUENCE LISTING (275 sequences)

SEQ ID NO: 1 - DNA, 30 bp, synthetic construct
  source 1..30: mol_type = other DNA
aatcatgctgctcagctggcaataatcaag

SEQ ID NO: 2 - DNA, 30 bp, synthetic construct
  source 1..30: mol_type = other DNA
aatcttccattcgagttgcgaaggaaagct

SEQ ID NO: 3 - DNA, 30 bp, synthetic construct
  source 1..30: mol_type = other DNA
acacacacacacacacacacacacacacac

SEQ ID NO: 4 - DNA, 30 bp, synthetic construct
  source 1..30: mol_type = other DNA
acctgcccgctcatctgcctgatactcgcc

SEQ ID NO: 5 - DNA, 30 bp, synthetic construct
  source 1..30: mol_type = other DNA
acgtgatcagtgcatgatcagtcacgtgat

SEQ ID NO: 6 - DNA, 30 bp, synthetic construct
  source 1..30: mol_type = other DNA
actgacctcgccgtcgtacctcgtgagaaa

SEQ ID NO: 7 - DNA, 30 bp, synthetic construct
  source 1..30: mol_type = other DNA
aggtgtcgcgcggctcagctggcgagtatc

SEQ ID NO: 8 - DNA, 30 bp, synthetic construct
  source 1..30: mol_type = other DNA
agtatcaggcagatgagcgggcaggtgtcg

SEQ ID NO: 9 - DNA, 30 bp, synthetic construct
  source 1..30: mol_type = other DNA
agtgcatgatagccacgtgatcagtgcatg

SEQ ID NO: 10 - DNA, 30 bp, synthetic construct
  source 1..30: mol_type = other DNA
atagccacgtgatcagtgcatgatcagtca

SEQ ID NO: 11 - DNA, 30 bp, synthetic construct
  source 1..30: mol_type = other DNA
atcacgtgactgatcatgcactgatcacgt

SEQ ID NO: 12 - DNA, 30 bp, synthetic construct
  source 1..30: mol_type = other DNA
atcaggcagatgagcgggcaggtgtcgcgc

SEQ ID NO: 13 - DNA, 30 bp, synthetic construct
  source 1..30: mol_type = other DNA
atcagtgcatgatagccacgtgatcagtgc

SEQ ID NO: 14 - DNA, 30 bp, synthetic construct
  source 1..30: mol_type = other DNA
atcatgcactgatcacgtgactgatcatgc

SEQ ID NO: 15 - DNA, 30 bp, synthetic construct
  source 1..30: mol_type = other DNA
atcatgcactgatcacgtggctatcatgca

SEQ ID NO: 16 - DNA, 30 bp, synthetic construct
  source 1..30: mol_type = other DNA
atcatgcactgatcacgtggctgatcatac

SEQ ID NO: 17 - DNA, 30 bp, synthetic construct
  source 1..30: mol_type = other DNA
atgatagccacgtgatcagtgcatgatcag

SEQ ID NO: 18 - DNA, 30 bp, synthetic construct
  source 1..30: mol_type = other DNA
atgatcagtcacgtgatcagtgcatgatca

SEQ ID NO: 19 - DNA, 30 bp, synthetic construct
  source 1..30: mol_type = other DNA
atgcactgatcacgtggctatcatgcactg

SEQ ID NO: 20 - DNA, 30 bp, synthetic construct
  source 1..30: mol_type = other DNA
atgcactgatcacgtggctgatcatacact

SEQ ID NO: 21 - DNA, 30 bp, synthetic construct
  source 1..30: mol_type = other DNA
cagctggcgagtatcaggcagatgagcggg

SEQ ID NO: 22 - DNA, 30 bp, synthetic construct
  source 1..30: mol_type = other DNA
catgatcagtcacgtgatctgtgcatgatc

SEQ ID NO: 23 - DNA, 30 bp, synthetic construct
  source 1..30: mol_type = other DNA
cgcggctcagctggcgagtatcaggcagat

SEQ ID NO: 24 - DNA, 30 bp, synthetic construct
  source 1..30: mol_type = other DNA
cgtgatcagtgcatgatagccacgtgatca

SEQ ID NO: 25 - DNA, 30 bp, synthetic construct
  source 1..30: mol_type = other DNA
ctcatctgcctgatactcgccagctgagcc

SEQ ID NO: 26 - DNA, 30 bp, synthetic construct
  source 1..30: mol_type = other DNA
ctgatcatgcactgatcacgtggctatcat

SEQ ID NO: 27 - DNA, 30 bp, synthetic construct
  source 1..30: mol_type = other DNA
cttccattcgagttgcgaaggaaagctggg

SEQ ID NO: 28 - DNA, 30 bp, synthetic construct
  source 1..30: mol_type = other DNA
gcatgatcagccacgtgatcagtgcatgat

SEQ ID NO: 29 - DNA, 30 bp, synthetic construct
  source 1..30: mol_type = other DNA
gcgagtatcaggcagatgagcgggcaggtg

SEQ ID NO: 30 - DNA, 30 bp, synthetic construct
  source 1..30: mol_type = other DNA
gctcagctggcgagtatcaggcagatgagc

SEQ ID NO: 31 - DNA, 30 bp, synthetic construct
  source 1..30: mol_type = other DNA
ggcaggtgtcgcgcggctcagctggcgagt

SEQ ID NO: 32 - DNA, 30 bp, synthetic construct
  source 1..30: mol_type = other DNA
gtgcatgatcagtcacgtgatctgtgcatg

SEQ ID NO: 33 - DNA, 30 bp, synthetic construct
  source 1..30: mol_type = other DNA
gtgtgtgtgtgtgtgtgtgtgtgtgtgtgt

SEQ ID NO: 34 - DNA, 30 bp, synthetic construct
  source 1..30: mol_type = other DNA
tcatgcactgatcacgtggctgatcatgca

SEQ ID NO: 35 - DNA, 30 bp, synthetic construct
  source 1..30: mol_type = other DNA
tgcatgatcagtcacgtgatcagtgcatga

SEQ ID NO: 36 - DNA, 30 bp, synthetic construct
  source 1..30: mol_type = other DNA
tgtcgcgcggctcagctggcgagtatcagg

SEQ ID NO: 37 - DNA, 30 bp, synthetic construct
  source 1..30: mol_type = other DNA
aatacccaaaacccaaaacccaaaacccaa

SEQ ID NO: 38 - DNA, 30 bp, synthetic construct
  source 1..30: mol_type = other DNA
aatccccaatccccaaaacccaaaacccca

SEQ ID NO: 39 - DNA, 30 bp, synthetic construct
  source 1..30: mol_type = other DNA
aattttaatactaataatgtaactaatatg

SEQ ID NO: 40 - DNA, 30 bp, synthetic construct
  source 1..30: mol_type = other DNA
agagcagagcagagcagagcagagcagagc

SEQ ID NO: 41 - DNA, 30 bp, synthetic construct
  source 1..30: mol_type = other DNA
ataataataataataataataataataata

SEQ ID NO: 42 - DNA, 30 bp, synthetic construct
  source 1..30: mol_type = other DNA
atattagttacattattagtattaaaatta

SEQ ID NO: 43 - DNA, 30 bp, synthetic construct
  source 1..30: mol_type = other DNA
attattattattattattattattattatt

SEQ ID NO: 44 - DNA, 30 bp, synthetic construct
  source 1..30: mol_type = other DNA
attgggcccaatccccaatccccaaacccc

SEQ ID NO: 45 - DNA, 30 bp, synthetic construct
  source 1..30: mol_type = other DNA
attggggattggggattggggagtggggat

SEQ ID NO: 46 - DNA, 30 bp, synthetic construct
  source 1..30: mol_type = other DNA
caatccccaaaacccaaaaccccaaacccc

SEQ ID NO: 47 - DNA, 30 bp, synthetic construct
  source 1..30: mol_type = other DNA
cactgactgcagtgataacactgactgcag

SEQ ID NO: 48 - DNA, 30 bp, synthetic construct
  source 1..30: mol_type = other DNA
ccaatccccaatccccaaaccccaatcccc

SEQ ID NO: 49 - DNA, 30 bp, synthetic construct
  source 1..30: mol_type = other DNA
ccaatccccaatccccaaacccccaaaccc

SEQ ID NO: 50 - DNA, 30 bp, synthetic construct
  source 1..30: mol_type = other DNA
ccaatccccaatccccaatacccaaaaccc

SEQ ID NO: 51 - DNA, 30 bp, synthetic construct
  source 1..30: mol_type = other DNA
cccaatccccaatccccaatccccaatacc

SEQ ID NO: 52 - DNA, 30 bp, synthetic construct
  source 1..30: mol_type = other DNA
ccccaatccccaaaacccaaaaccccaaac

SEQ ID NO: 53 - DNA, 30 bp, synthetic construct
  source 1..30: mol_type = other DNA
ccccaatccccaatccccaaaacccaaaac

SEQ ID NO: 54 - DNA, 30 bp, synthetic construct
  source 1..30: mol_type = other DNA
cttatacacatctcgagcccacgagacact

SEQ ID NO: 55 - DNA, 30 bp, synthetic construct
  source 1..30: mol_type = other DNA
cttatacacatctcgagcccacgagaccta

SEQ ID NO: 56 - DNA, 30 bp, synthetic construct
  source 1..30: mol_type = other DNA
cttatacacatctcgagcccacgagacgct

SEQ ID NO: 57 - DNA, 30 bp, synthetic construct
  source 1..30: mol_type = other DNA
gactgcagtgataacactgactgcagtgat

SEQ ID NO: 58 - DNA, 30 bp, synthetic construct
  source 1..30: mol_type = other DNA
gataacactgactgcagtgataacactgac

SEQ ID NO: 59 - DNA, 30 bp, synthetic construct
  source 1..30: mol_type = other DNA
gattggggattggggagtggggattgggga

SEQ ID NO: 60 - DNA, 30 bp, synthetic construct
  source 1..30: mol_type = other DNA
gggattggggattggggattggggagtggg

SEQ ID NO: 61 - DNA, 30 bp, synthetic construct
  source 1..30: mol_type = other DNA
ggggattggggattggggagtggggattgg

SEQ ID NO: 62 - DNA, 30 bp, synthetic construct
  source 1..30: mol_type = other DNA
tattggggattggggattggggattgggga

SEQ ID NO: 63 - DNA, 30 bp, synthetic construct
  source 1..30: mol_type = other DNA
tgcttgcttgcttgcttgcttgcttgcttg

SEQ ID NO: 64 - DNA, 30 bp, synthetic construct
  source 1..30: mol_type = other DNA
ttggggagtggggattggggattggggatt

SEQ ID NO: 65 - DNA, 30 bp, synthetic construct; identical to SEQ ID NO: 5
  source 1..30: mol_type = other DNA

SEQ ID NO: 66 - DNA, 30 bp, synthetic construct; identical to SEQ ID NO: 7
  source 1..30: mol_type = other DNA

SEQ ID NO: 67 - DNA, 30 bp, synthetic construct; identical to SEQ ID NO: 8
  source 1..30: mol_type = other DNA

SEQ ID NO: 68 - DNA, 30 bp, synthetic construct; identical to SEQ ID NO: 9
  source 1..30: mol_type = other DNA

SEQ ID NO: 69 - DNA, 30 bp, synthetic construct; identical to SEQ ID NO: 10
  source 1..30: mol_type = other DNA

SEQ ID NO: 70 - DNA, 30 bp, synthetic construct; identical to SEQ ID NO: 13
  source 1..30: mol_type = other DNA

SEQ ID NO: 71 - DNA, 30 bp, synthetic construct; identical to SEQ ID NO: 14
  source 1..30: mol_type = other DNA

SEQ ID NO: 72 - DNA, 30 bp, synthetic construct; identical to SEQ ID NO: 15
  source 1..30: mol_type = other DNA

SEQ ID NO: 73 - DNA, 30 bp, synthetic construct; identical to SEQ ID NO: 17
  source 1..30: mol_type = other DNA

SEQ ID NO: 74 - DNA, 30 bp, synthetic construct; identical to SEQ ID NO: 18
  source 1..30: mol_type = other DNA

SEQ ID NO: 75 - DNA, 30 bp, synthetic construct; identical to SEQ ID NO: 19
  source 1..30: mol_type = other DNA

SEQ ID NO: 76 - DNA, 30 bp, synthetic construct
  source 1..30: mol_type = other DNA
attggggattggggattggggattggggat

SEQ ID NO: 77 - DNA, 30 bp, synthetic construct; identical to SEQ ID NO: 21
  source 1..30: mol_type = other DNA

SEQ ID NO: 78 - DNA, 30 bp, synthetic construct; identical to SEQ ID NO: 23
  source 1..30: mol_type = other DNA

SEQ ID NO: 79 - DNA, 30 bp, synthetic construct; identical to SEQ ID NO: 24
  source 1..30: mol_type = other DNA

SEQ ID NO: 80 - DNA, 30 bp, synthetic construct; identical to SEQ ID NO: 25
  source 1..30: mol_type = other DNA

SEQ ID NO: 81 - DNA, 30 bp, synthetic construct; identical to SEQ ID NO: 28
  source 1..30: mol_type = other DNA

SEQ ID NO: 82 - DNA, 30 bp, synthetic construct; identical to SEQ ID NO: 29
  source 1..30: mol_type = other DNA

SEQ ID NO: 83 - DNA, 30 bp, synthetic construct; identical to SEQ ID NO: 30
  source 1..30: mol_type = other DNA

SEQ ID NO: 84 - DNA, 30 bp, synthetic construct; identical to SEQ ID NO: 31
  source 1..30: mol_type = other DNA

SEQ ID NO: 85 - DNA, 30 bp, synthetic construct
  source 1..30: mol_type = other DNA
gggattggggattggggattggggattggg

SEQ ID NO: 86 - DNA, 30 bp, synthetic construct
  source 1..30: mol_type = other DNA
gtgcatgatcagtcacgtgatcagtgcatg

SEQ ID NO: 87 - DNA, 30 bp, synthetic construct; identical to SEQ ID NO: 33
  source 1..30: mol_type = other DNA

SEQ ID NO: 88 - DNA, 30 bp, synthetic construct; identical to SEQ ID NO: 34
  source 1..30: mol_type = other DNA

SEQ ID NO: 89 - DNA, 30 bp, synthetic construct; identical to SEQ ID NO: 36
  source 1..30: mol_type = other DNA

SEQ ID NO: 90 - DNA, 30 bp, synthetic construct
  source 1..30: mol_type = other DNA
aaaggtacgaaaattttagctaatcacaac

SEQ ID NO: 91 - DNA, 30 bp, synthetic construct
  source 1..30: mol_type = other DNA
accttgcaaaggtacgaaaattttagctaa

SEQ ID NO: 92 - DNA, 30 bp, synthetic construct
  source 1..30: mol_type = other DNA
acgcgtggacgcgtggacgcgtggacgcgt

SEQ ID NO: 93 - DNA, 30 bp, synthetic construct; identical to SEQ ID NO: 41
  source 1..30: mol_type = other DNA

SEQ ID NO: 94 - DNA, 30 bp, synthetic construct
  source 1..30: mol_type = other DNA
atgaccttgcaaaggtacgaaaattttagc

SEQ ID NO: 95 - DNA, 30 bp, synthetic construct
  source 1..30: mol_type = other DNA
cgtggacgcgtggacgcgtggacgcgtgga

SEQ ID NO: 96 - DNA, 30 bp, synthetic construct; identical to SEQ ID NO: 55
  source 1..30: mol_type = other DNA

SEQ ID NO: 97 - DNA, 30 bp, synthetic construct; identical to SEQ ID NO: 56
  source 1..30: mol_type = other DNA

SEQ ID NO: 98 - DNA, 30 bp, synthetic construct
  source 1..30: mol_type = other DNA
gacgcatgacgcatgacgcatgacgcatga

SEQ ID NO: 99 - DNA, 30 bp, synthetic construct
  source 1..30: mol_type = other DNA
gccaagctgttcttggcgtaagatgcaatg

SEQ ID NO: 100 - DNA, 30 bp, synthetic construct
  source 1..30: mol_type = other DNA
gcgtaagatgcaatggctgagaacttgact

SEQ ID NO: 101 - DNA, 30 bp, synthetic construct
  source 1..30: mol_type = other DNA
gctgagaacttgactttcaagagttctttt

SEQ ID NO: 102 - DNA, 30 bp, synthetic construct
  source 1..30: mol_type = other DNA
gctgttcttggcgtaagatgcaatggctga

SEQ ID NO: 103 - DNA, 30 bp, synthetic construct
  source 1..30: mol_type = other DNA
gttcttggcgtaagatgcaatggctgagaa

SEQ ID NO: 104 - DNA, 30 bp, synthetic construct
  source 1..30: mol_type = other DNA
gttgagagttgagagttgagagttgagagt

SEQ ID NO: 105 - DNA, 30 bp, synthetic construct
  source 1..30: mol_type = other DNA
gttgatgaccttgcaaaggtacgaaaattt

SEQ ID NO: 106 - DNA, 30 bp, synthetic construct
  source 1..30: mol_type = other DNA
taagatgcaatggctgagaacttgactttc

SEQ ID NO: 107 - DNA, 30 bp, synthetic construct
  source 1..30: mol_type = other DNA
taggccaagctgttcttggcgtaagatgca

SEQ ID NO: 108 - DNA, 30 bp, synthetic construct
  source 1..30: mol_type = other DNA
tcatgcgtcatgcgtcatgcgtcatgcgtc

SEQ ID NO: 109 - DNA, 30 bp, synthetic construct
  source 1..30: mol_type = other DNA
tctcttatacacatctacgctgccgacgac

SEQ ID NO: 110 - DNA, 30 bp, synthetic construct
  source 1..30: mol_type = other DNA
tcttatacacatctccagcccacgagactt

SEQ ID NO: 111 - DNA, 30 bp, synthetic construct
  source 1..30: mol_type = other DNA
tcttatacacatctcgagcccacgagactt

SEQ ID NO: 112 - DNA, 30 bp, synthetic construct
  source 1..30: mol_type = other DNA
tcttatacacatcttgacgctgccgacgac

SEQ ID NO: 113 - DNA, 30 bp, synthetic construct
  source 1..30: mol_type = other DNA
tgcaaaggtacgaaaattttagctaatcac

SEQ ID NO: 114 - DNA, 30 bp, synthetic construct
  source 1..30: mol_type = other DNA
tgcaatggctgagaacttgactttcaagag

SEQ ID NO: 115 - DNA, 30 bp, synthetic construct
  source 1..30: mol_type = other DNA
tgtcaagcggcaaccgatcggttacgctga

SEQ ID NO: 116 - DNA, 30 bp, synthetic construct
  source 1..30: mol_type = other DNA
ttatctcattgcttttcacctcacacattt

SEQ ID NO: 117 - DNA, 30 bp, synthetic construct
  source 1..30: mol_type = other DNA
ttcaagagttcttttctctttctgattgcc

SEQ ID NO: 118 - DNA, 30 bp, synthetic construct
  source 1..30: mol_type = other DNA
ttcacctcacacatttcagtgtcaagcggc

SEQ ID NO: 119 - DNA, 30 bp, synthetic construct
  source 1..30: mol_type = other DNA
ttcagtgtcaagcggcaaccgatcggttac

SEQ ID NO: 120 - DNA, 30 bp, synthetic construct
  source 1..30: mol_type = other DNA
ttgactttcaagagttcttttctctttctg

SEQ ID NO: 121 - DNA, 30 bp, synthetic construct
  source 1..30: mol_type = other DNA
ttgcttttcacctcacacatttcagtgtca

SEQ ID NO: 122 - DNA, 30 bp, synthetic construct
  source 1..30: mol_type = other DNA
ttggcgtaagatgcaatggctgagaacttg

SEQ ID NO: 123 - DNA, 30 bp, synthetic construct
  source 1..30: mol_type = other DNA
aaacatgggcaggcctatgaaacccaccgc

SEQ ID NO: 124 - DNA, 30 bp, synthetic construct
  source 1..30: mol_type = other DNA
aaagagaggtgagaaacatgggcaggccta

SEQ ID NO: 125 - DNA, 30 bp, synthetic construct
  source 1..30: mol_type = other DNA
aaattaatgtttatatatgttaaattaatg

SEQ ID NO: 126 - DNA, 30 bp, synthetic construct
  source 1..30: mol_type = other DNA
aacgctgtacaagaagcgcctgaacaccga

SEQ ID NO: 127 - DNA, 30 bp, synthetic construct
  source 1..30: mol_type = other DNA
acgcatgacgcatgacgcatgacgcatgac

SEQ ID NO: 128 - DNA, 30 bp, synthetic construct
  source 1..30: mol_type = other DNA
atatgttaaattaatgtttatatatgttaa

SEQ ID NO: 129 - DNA, 30 bp, synthetic construct
  source 1..30: mol_type = other DNA
atgacgcatgacgcatgacgcatgacgcat

SEQ ID NO: 130 - DNA, 30 bp, synthetic construct
  source 1..30: mol_type = other DNA
atgcgtcatgcgtcatgcgtcatgcgtcat

SEQ ID NO: 131 - DNA, 30 bp, synthetic construct
  source 1..30: mol_type = other DNA
atgggcaggcctatgaaacccaccgcagtc

SEQ ID NO: 132 - DNA, 30 bp, synthetic construct
  source 1..30: mol_type = other DNA
caggcctatgaaacccaccgcagtcaagaa

SEQ ID NO: 133 - DNA, 30 bp, synthetic construct
  source 1..30: mol_type = other DNA
ccagaccctcagcgacatcggaacgaccgc

SEQ ID NO: 134 - DNA, 30 bp, synthetic construct
  source 1..30: mol_type = other DNA
cgtcatgcgtcatgcgtcatgcgtcatgcg

SEQ ID NO: 135 - DNA, 30 bp, synthetic construct
  source 1..30: mol_type = other DNA
ctctgctctgctctgctctgctctgctctg

SEQ ID NO: 136 - DNA, 30 bp, synthetic construct
  source 1..30: mol_type = other DNA
ctcttatacacatctcgagcccacgagaca

SEQ ID NO: 137 - DNA, 30 bp, synthetic construct
  source 1..30: mol_type = other DNA
gagaaacatgggcaggcctatgaaacccac

SEQ ID NO: 138 - DNA, 30 bp, synthetic construct
  source 1..30: mol_type = other DNA
ggtgagaaacatgggcaggcctatgaaacc

SEQ ID NO: 139 - DNA, 30 bp, synthetic construct
  source 1..30: mol_type = other DNA
gttaaattaatgtttatatatgttaaatta

SEQ ID NO: 140 - DNA, 30 bp, synthetic construct
  source 1..30: mol_type = other DNA
tctttttctttttctttttctttttctttt

SEQ ID NO: 141 - DNA, 30 bp, synthetic construct
  source 1..30: mol_type = other DNA
ttttctttttctttttctttttctttttct

SEQ ID NO: 142 - DNA, 30 bp, synthetic construct
  source 1..30: mol_type = other DNA
aaaattagataaatttaaagaagttaaaga

SEQ ID NO: 143 - DNA, 30 bp, synthetic construct
  source 1..30: mol_type = other DNA
aacattattagtattaaaattagataaatt

SEQ ID NO: 144 - DNA, 30 bp, synthetic construct
  source 1..30: mol_type = other DNA
aataataataataataataataataataat

SEQ ID NO: 145 - DNA, 30 bp, synthetic construct
  source 1..30: mol_type = other DNA
aatccccaatccccaaaacccaaaacccaa

SEQ ID NO: 146 - DNA, 30 bp, synthetic construct
  source 1..30: mol_type = other DNA
aatggggattggggattggggattggggat

SEQ ID NO: 147 - DNA, 30 bp, synthetic construct
  source 1..30: mol_type = other DNA
aattggggattggggattggggattgggga

SEQ ID NO: 148 - DNA, 30 bp, synthetic construct
  source 1..30: mol_type = other DNA
agataaatttaaagaagttaaagaagaaca

SEQ ID NO: 149 - DNA, 30 bp, synthetic construct
  source 1..30: mol_type = other DNA
agtattaaaattagataaatttaaagaagt

SEQ ID NO: 150 - DNA, 30 bp, synthetic construct
  source 1..30: mol_type = other DNA
attaaaattagataaatttaaagaagttaa

SEQ ID NO: 151 - DNA, 30 bp, synthetic construct
  source 1..30: mol_type = other DNA
attagataaatttaaagaagttaaagaaga

SEQ ID NO: 152 - DNA, 30 bp, synthetic construct
  source 1..30: mol_type = other DNA
attagtattaaaattagataaatttaaaga

SEQ ID NO: 153 - DNA, 30 bp, synthetic construct
  source 1..30: mol_type = other DNA
attattagtattaaaattagataaatttaa

SEQ ID NO: 154 - DNA, 30 bp, synthetic construct; identical to SEQ ID NO: 43
  source 1..30: mol_type = other DNA

SEQ ID NO: 155 - DNA, 30 bp, synthetic construct; identical to SEQ ID NO: 44
  source 1..30: mol_type = other DNA

SEQ ID NO: 156 - DNA, 30 bp, synthetic construct
  source 1..30: mol_type = other DNA
attggggattggggattggggattgggccc

SEQ ID NO: 157 - DNA, 30 bp, synthetic construct
  source 1..30: mol_type = other DNA
ccaatccccaaaacccaaaaccccaaaccc

SEQ ID NO: 158 - DNA, 30 bp, synthetic construct; identical to SEQ ID NO: 50
  source 1..30: mol_type = other DNA

SEQ ID NO: 159 - DNA, 30 bp, synthetic construct
  source 1..30: mol_type = other DNA
ccaatccccaatccccaatccccaatcccc

SEQ ID NO: 160 - DNA, 30 bp, synthetic construct
  source 1..30: mol_type = other DNA
cccaatccccaatccccaaaacccaaaacc

SEQ ID NO: 161 - DNA, 30 bp, synthetic construct
  source 1..30: mol_type = other DNA
gattggggattggggattggggattggggg

SEQ ID NO: 162 - DNA, 30 bp, synthetic construct
  source 1..30: mol_type = other DNA
gggattggggagtggggattggggattggg

SEQ ID NO: 163 - DNA, 30 bp, synthetic construct
  source 1..30: mol_type = other DNA
ggggattggggattggggattggggattgg

SEQ ID NO: 164 - DNA, 30 bp, synthetic construct
  source 1..30: mol_type = other DNA
taaatttaaagaagttaaagaagaacaatt

SEQ ID NO: 165 - DNA, 30 bp, synthetic construct
  source 1..30: mol_type = other DNA
tccccaatccccaatccccaatccccatta

SEQ ID NO: 166 - DNA, 30 bp, synthetic construct
  source 1..30: mol_type = other DNA
tcttatacacatctcgagcccacgagacga

SEQ ID NO: 167 - DNA, 30 bp, synthetic construct
  source 1..30: mol_type = other DNA
tggggattggggattggggagtggggattg

SEQ ID NO: 168 - DNA, 30 bp, synthetic construct
  source 1..30: mol_type = other DNA
ttggggattggggagtggggattggggatt

SEQ ID NO: 169 - DNA, 30 bp, synthetic construct
  source 1..30: mol_type = other DNA
ttggggattggggattggggattggggcca

SEQ ID NO: 170 - DNA, 30 bp, synthetic construct
  source 1..30: mol_type = other DNA
aaacaccatatatattgagaaagagaggtg

SEQ ID NO: 171 - DNA, 30 bp, synthetic construct; identical to SEQ ID NO: 123
  source 1..30: mol_type = other DNA

SEQ ID NO: 172 - DNA, 30 bp, synthetic construct; identical to SEQ ID NO: 125
  source 1..30: mol_type = other DNA

SEQ ID NO: 173 - DNA, 30 bp, synthetic construct
  source 1..30: mol_type = other DNA
aaatttaaagaagttaaagaagaacaatta

SEQ ID NO: 174 - DNA, 30 bp, synthetic construct
  source 1..30: mol_type = other DNA
aaatttatctaattttaatactaataatgt

SEQ ID NO: 175 - DNA, 30 bp, synthetic construct
  source 1..30: mol_type = other DNA
aacttctttaaatttatctaattttaatac

SEQ ID NO: 176 - DNA, 30 bp, synthetic construct
  source 1..30: mol_type = other DNA
aagaagaagaagaagaagaagttgaacatg

SEQ ID NO: 177 - DNA, 30 bp, synthetic construct
  source 1..30: mol_type = other DNA
aagaagaagaagaagaagttgaacatgaag

SEQ ID NO: 178 - DNA, 30 bp, synthetic construct
  source 1..30: mol_type = other DNA
aattttaatactaataatgttaataatatg

SEQ ID NO: 179 - DNA, 30 bp, synthetic construct
  source 1..30: mol_type = other DNA
aattttaatactaataatgttactgatatg

SEQ ID NO: 180 - DNA, 30 bp, synthetic construct
  source 1..30: mol_type = other DNA
acactaaacaccatatatattgagaaagag

SEQ ID NO: 181 - DNA, 30 bp, synthetic construct
  source 1..30: mol_type = other DNA
accatatatattgagaaagagaggtgagaa

SEQ ID NO: 182 - DNA, 30 bp, synthetic construct; identical to SEQ ID NO: 148
  source 1..30: mol_type = other DNA

SEQ ID NO: 183 - DNA, 30 bp, synthetic construct
  source 1..30: mol_type = other DNA
aggcctatgaaacccaccgcagtcaagaag

SEQ ID NO: 184 - DNA, 30 bp, synthetic construct
  source 1..30: mol_type = other DNA
ataaatggggattggggattggggattggg

SEQ ID NO: 185 - DNA, 30 bp, synthetic construct
  source 1..30: mol_type = other DNA
atctaattttaatactaataatgttaataa

SEQ ID NO: 186 - DNA, 30 bp, synthetic construct; identical to SEQ ID NO: 130
  source 1..30: mol_type = other DNA

SEQ ID NO: 187 - DNA, 30 bp, synthetic construct
  source 1..30: mol_type = other DNA
atggggattggggattggggattggggatt

SEQ ID NO: 188 - DNA, 30 bp, synthetic construct; identical to SEQ ID NO: 43
  source 1..30: mol_type = other DNA

SEQ ID NO: 189 - DNA, 30 bp, synthetic construct; identical to SEQ ID NO: 159
  source 1..30: mol_type = other DNA

SEQ ID NO: 190 - DNA, 30 bp, synthetic construct; identical to SEQ ID NO: 134
  source 1..30: mol_type = other DNA

SEQ ID NO: 191 - DNA, 30 bp, synthetic construct; identical to SEQ ID NO: 135
  source 1..30: mol_type = other DNA

SEQ ID NO: 192 - DNA, 30 bp, synthetic construct
  source 1..30: mol_type = other DNA
ctcttatacacatctcgagcccacgagacg

SEQ ID NO: 193 - DNA, 30 bp, synthetic construct
  source 1..30: mol_type = other DNA
cttatacacatctcgagcccacgagacaac

SEQ ID NO: 194 - DNA, 30 bp, synthetic construct
  source 1..30: mol_type = other DNA
cttatacacatctcgagcccacgagactgt

SEQ ID NO: 195 - DNA, 30 bp, synthetic construct
  source 1..30: mol_type = other DNA
ctttaaatttatctaattttaatactaata

SEQ ID NO: 196 - DNA, 30 bp, synthetic construct
  source 1..30: mol_type = other DNA
ctttaacttctttaaatttatctaatttta

SEQ ID NO: 197 - DNA, 30 bp, synthetic construct
  source 1..30: mol_type = other DNA
gattggggattggggattggggattgggga

SEQ ID NO: 198 - DNA, 30 bp, synthetic construct
  source 1..30: mol_type = other DNA
tttatctaattttaatactaataatgttaa

SEQ ID NO: 199 - DNA, 30 bp, synthetic construct
  source 1..30: mol_type = other DNA
aaacgcctcaggaggcttgactcccttgag

SEQ ID NO: 200 - DNA, 30 bp, synthetic construct
  source 1..30: mol_type = other DNA
aagaagaagaagaagaagaagaagaagaag

SEQ ID NO: 201 - DNA, 30 bp, synthetic construct
  source 1..30: mol_type = other DNA
aggtacgacggcgaggtcagtgagcctctc

SEQ ID NO: 202 - DNA, 30 bp, synthetic construct; identical to SEQ ID NO: 9
  source 1..30: mol_type = other DNA

SEQ ID NO: 203 - DNA, 30 bp, synthetic construct; identical to SEQ ID NO: 13
  source 1..30: mol_type = other DNA

SEQ ID NO: 204 - DNA, 30 bp, synthetic construct; identical to SEQ ID NO: 14
  source 1..30: mol_type = other DNA

SEQ ID NO: 205 - DNA, 30 bp, synthetic construct
  source 1..30: mol_type = other DNA
atctcgcgacctctctccaaacgcctcagg

SEQ ID NO: 206 - DNA, 30 bp, synthetic construct
  source 1..30: mol_type = other DNA
atgcactgatcacgtggctgatcatgcact

SEQ ID NO: 207 - DNA, 30 bp, synthetic construct
  source 1..30: mol_type = other DNA
cacacacacacacacacacacacacacaca

SEQ ID NO: 208 - DNA, 30 bp, synthetic construct
  source 1..30: mol_type = other DNA
caggaggcttgactcccttgagtccaccca

SEQ ID NO: 209 - DNA, 30 bp, synthetic construct
  source 1..30: mol_type = other DNA
catgatagccacgtgatcagtgcatgatca

SEQ ID NO: 210 - DNA, 30 bp, synthetic construct
  source 1..30: mol_type = other DNA
cctcaggaggcttgactcccttgagtccac

SEQ ID NO: 211 - DNA, 30 bp, synthetic construct
  source 1..30: mol_type = other DNA
cctctctccaaacgcctcaggaggcttgac

SEQ ID NO: 212 - DNA, 30 bp, synthetic construct
  source 1..30: mol_type = other DNA
cggctcggctcggctcggctcggctcggct

SEQ ID NO: 213 - DNA, 30 bp, synthetic construct; identical to SEQ ID NO: 24
  source 1..30: mol_type = other DNA

SEQ ID NO: 214 - DNA, 30 bp, synthetic construct
  source 1..30: mol_type = other DNA
ctccaaacgcctcaggaggcttgactccct

SEQ ID NO: 215 - DNA, 30 bp, synthetic construct; identical to SEQ ID NO: 26
  source 1..30: mol_type = other DNA

SEQ ID NO: 216 - DNA, 30 bp, synthetic construct
  source 1..30: mol_type = other DNA
gcgacctctctccaaacgcctcaggaggct

SEQ ID NO: 217 - DNA, 30 bp, synthetic construct
  source 1..30: mol_type = other DNA
gtccacccagtgagctccaagagatacccg

SEQ ID NO: 218 - DNA, 30 bp, synthetic construct
  source 1..30: mol_type = other DNA
tcttatacacatctcgagcccacgagactc

SEQ ID NO: 219 - DNA, 30 bp, synthetic construct
  source 1..30: mol_type = other DNA
aacctggtggaaaccccgagcctacaacag

SEQ ID NO: 220 - DNA, 30 bp, synthetic construct
  source 1..30: mol_type = other DNA
aagtgtgggtatacgccccatagtgggcgc

SEQ ID NO: 221 - DNA, 30 bp, synthetic construct
  source 1..30: mol_type = other DNA
acttatcaagcttaactggctagtgatata

SEQ ID NO: 222 - DNA, 30 bp, synthetic construct
  source 1..30: mol_type = other DNA
agcccgccttgcagcagaagcgccactgag

SEQ ID NO: 223 - DNA, 30 bp, synthetic construct
  source 1..30: mol_type = other DNA
atttccaagaacggacctcgcacataaccg

SEQ ID NO: 224 - DNA, 30 bp, synthetic construct
  source 1..30: mol_type = other DNA
cactaaaaagtaaagtcgatcagcctgaat

SEQ ID NO: 225 - DNA, 30 bp, synthetic construct
  source 1..30: mol_type = other DNA
cattgtacaacgaggatgaagcgttacaca

SEQ ID NO: 226 - DNA, 30 bp, synthetic construct
  source 1..30: mol_type = other DNA
cccccttgttggtgcaattaccatagtaaa

SEQ ID NO: 227 - DNA, 30 bp, synthetic construct
  source 1..30: mol_type = other DNA
ccgcaaagcggactcgtagcgatggaaatg

SEQ ID NO: 228 - DNA, 30 bp, synthetic construct
  source 1..30: mol_type = other DNA
cggagagaactacttccgtattttaccgca

SEQ ID NO: 229 - DNA, 30 bp, synthetic construct
  source 1..30: mol_type = other DNA
cgggatgcatacatcgcctacggtaagcac

SEQ ID NO: 230 - DNA, 30 bp, synthetic construct
  source 1..30: mol_type = other DNA
cgtgcagggtgctggaagagcacttaaagc

SEQ ID NO: 231 - DNA, 30 bp, synthetic construct
  source 1..30: mol_type = other DNA
ctcggcccgtacaggcacggggcaccagtg

SEQ ID NO: 232 - DNA, 30 bp, synthetic construct
  source 1..30: mol_type = other DNA
ctgaagcaatacaggcgtgtgaccgcgtaa

SEQ ID NO: 233 - DNA, 30 bp, synthetic construct
  source 1..30: mol_type = other DNA
ctgacaccgcccgtgacaatgagggtcctg

SEQ ID NO: 234 - DNA, 30 bp, synthetic construct
  source 1..30: mol_type = other DNA
gagccacctagttaataaatccctctcacc

SEQ ID NO: 235 - DNA, 30 bp, synthetic construct
  source 1..30: mol_type = other DNA
gagggagcccatatatccttgtttcttata

SEQ ID NO: 236 - DNA, 30 bp, synthetic construct
  source 1..30: mol_type = other DNA
gcgggagtttcggacggatgtggtccccta

SEQ ID NO: 237 - DNA, 30 bp, synthetic construct
  source 1..30: mol_type = other DNA
gcggtgggaagggccgaagacgaattagaa

SEQ ID NO: 238 - DNA, 30 bp, synthetic construct
  source 1..30: mol_type = other DNA
gctaggtcatagagttacatgctcaccggc

SEQ ID NO: 239 - DNA, 30 bp, synthetic construct
  source 1..30: mol_type = other DNA
ggctcgatggtctggctcatgggttcatca

SEQ ID NO: 240 - DNA, 30 bp, synthetic construct
  source 1..30: mol_type = other DNA
ggtctccaaaactccaagtagtctacccac

SEQ ID NO: 241 - DNA, 30 bp, synthetic construct
  source 1..30: mol_type = other DNA
gtagcgagtctacgttgctctgtcgatgcg

SEQ ID NO: 242 - DNA, 30 bp, synthetic construct
  source 1..30: mol_type = other DNA
tagattctaaacatcttacggcgattttct

SEQ ID NO: 243 - DNA, 30 bp, synthetic construct
  source 1..30: mol_type = other DNA
tcccagagttctgtggggctttttgtaaaa

SEQ ID NO: 244 - DNA, 30 bp, synthetic construct
  source 1..30: mol_type = other DNA
tcgacctcgtatggttctcggcagctgcga

SEQ ID NO: 245 - DNA, 30 bp, synthetic construct
  source 1..30: mol_type = other DNA
tcggtaaataaggcccccatgataagtact

SEQ ID NO: 246 - DNA, 30 bp, synthetic construct
  source 1..30: mol_type = other DNA
tcgtgcagccactaccgacttaggggcctg

SEQ ID NO: 247 - DNA, 30 bp, synthetic construct
  source 1..30: mol_type = other DNA
tggtcatcgcctacacgttagcagtcaaca

SEQ ID NO: 248 - DNA, 30 bp, synthetic construct
  source 1..30: mol_type = other DNA
ttccatgaggtggctagacgttccgcaaat

SEQ ID NO: 249 - DNA, 30 bp, synthetic construct
  source 1..30: mol_type = other DNA
ttgaaataccgaggcgtgtgggtaagtgat

SEQ ID NO: 250 - DNA, 30 bp, synthetic construct
  source 1..30: mol_type = other DNA
ttgtcctagccattatctcgaaaaggtctt

SEQ ID NO: 251 - DNA, 30 bp, synthetic construct
  source 1..30: mol_type = other DNA
aaagtctgtccgacttgaatccctgttgtt

SEQ ID NO: 252 - DNA, 30 bp, synthetic construct
  source 1..30: mol_type = other DNA
acaatccccaatccccaatccccatttaat

SEQ ID NO: 253 - DNA, 30 bp, synthetic construct
  source 1..30: mol_type = other DNA
acacaccttggccagcaacacaccacgcca

SEQ ID NO: 254 - DNA, 30 bp, synthetic construct
  source 1..30: mol_type = other DNA
acacaggaccgccctgccagtaggtttgca

SEQ ID NO: 255 - DNA, 30 bp, synthetic construct
  source 1..30: mol_type = other DNA
acacaggcagcaccaggagatgcgggcaga

SEQ ID NO: 256 - DNA, 30 bp, synthetic construct
  source 1..30: mol_type = other DNA
ccacatggaagaagcgtctcggtatataat

SEQ ID NO: 257 - DNA, 30 bp, synthetic construct
  source 1..30: mol_type = other DNA
ccaccacaatggcgcctttcatttttgcca

SEQ ID NO: 258 - DNA, 30 bp, synthetic construct
  source 1..30: mol_type = other DNA
ccaccgcgcacgatggtctccacgggccat

SEQ ID NO: 259 - DNA, 30 bp, synthetic construct
  source 1..30: mol_type = other DNA
ccacctgtgtcggtttacggtacgggctgc

SEQ ID NO: 260 - DNA, 30 bp, synthetic construct
  source 1..30: mol_type = other DNA
ccacgtgcaggtgagattatccgtcaggga

SEQ ID NO: 261 - DNA, 30 bp, synthetic construct
  source 1..30: mol_type = other DNA
ccactctttaaattgagaattgaaatttga

SEQ ID NO: 262 - DNA, 30 bp, synthetic construct
  source 1..30: mol_type = other DNA
ccagagcccatcaagtatgtggacaatctg

SEQ ID NO: 263 - DNA, 30 bp, synthetic construct
  source 1..30: mol_type = other DNA
cgaagaggtcggcggccgaagggaaagcca

SEQ ID NO: 264 - DNA, 30 bp, synthetic construct
  source 1..30: mol_type = other DNA
ctccgagcccaccagacccgcggttctatc

SEQ ID NO: 265 - DNA, 30 bp, synthetic construct
  source 1..30: mol_type = other DNA
gaacgcccatcttgtcgaggatgatacctg

SEQ ID NO: 266 - DNA, 30 bp, synthetic construct
  source 1..30: mol_type = other DNA
gaacgtcgaagaggtcggcggccgaaggga

SEQ ID NO: 267 - DNA, 30 bp, synthetic construct
  source 1..30: mol_type = other DNA
gactctctaattcgttaattctcactctag

SEQ ID NO: 268 - DNA, 30 bp, synthetic construct
  source 1..30: mol_type = other DNA
ggattccctggcacaggccagcgtgagccc

SEQ ID NO: 269 - DNA, 30 bp, synthetic construct
  source 1..30: mol_type = other DNA
ggccgttacacagattttcttgcctgtggc

SEQ ID NO: 270 - DNA, 30 bp, synthetic construct
  source 1..30: mol_type = other DNA
ggctgttgagtgcggttactggcacctgtg

SEQ ID NO: 271 - DNA, 30 bp, synthetic construct
  source 1..30: mol_type = other DNA
ggtccaaggtaggtccaaggtaggtccaag

SEQ ID NO: 272 - DNA, 30 bp, synthetic construct
  source 1..30: mol_type = other DNA
tgtgaaattccgccagaaaggcgtggaata

SEQ ID NO: 273 - DNA, 30 bp, synthetic construct
  source 1..30: mol_type = other DNA
tgtgatgagattgagatgaaggacaaggag

SEQ ID NO: 274 - DNA, 30 bp, synthetic construct
  source 1..30: mol_type = other DNA
ttccgtccagtcctcgccgctcatcgcctt

SEQ ID NO: 275 - DNA, 30 bp, synthetic construct
  source 1..30: mol_type = other DNA
ttcgaacgtgagcgagctcgatgtgaaaca